(19) United States
(12) Patent Application Publication
(10) Pub. No.: US 2026/0297212 A1
(43) Pub. Date: Oct. 1, 2026

(54) ANTI-DLL3 ANTIBODIES AND METHODS OF USE

(71) Applicant: Mythic Therapeutics, Inc., Waltham, MA (US)

(72) Inventors: Gholamreza HASSANZADEH GHASSABEH, Ghent (BE); Lorena LERNER, Andover, MA (US); Sonia FEAU, Chestnut Hill, MA (US); Christophe QUÉVA, Cambridge, MA (US)

(21) Appl. No.: 19/704,732

(22) Filed: Jun. 11, 2026

Related U.S. Application Data
(63) Continuation of application No. 17/997,907, filed on May 5, 2021, now Pat. No. 12,686,725.
(60) Provisional application No. 63/020,444, filed on May 5, 2020.

(51) Int. Cl. C07K 16/30; A61P 35/00; C07K 16/28; C12N 15/63

(52) U.S. Cl. CPC C07K 16/3023; A61P 35/00; C07K 16/2809; C12N 15/63; C07K 2317/31; C07K 2317/52; C07K 2317/565

(57) ABSTRACT

The present disclosure provides anti-DLL3 binding constructs, such as anti-DLL3 single domain antibodies, as well as polynucleotide encoding the same. Further provided are multispecific binding constructs comprising the DLL3 binding domains described herein and polynucleotides encoding the same. Methods of production of the anti-DLL3 binding constructs and their use in the treatment of cancer are also provided herein.

### CROSS-REFERENCE TO RELATED APPLICATIONS

The present application is a continuation application of and claims priority to U.S. National Phase application Ser. No. 17/997,907, filed on Nov. 3, 2022, which claims priority to International Application No. PCT/US2021/030836, filed on May 5, 2021, which claims priority to U.S. Provisional Application No. 63/020,444, filed May 5, 2020, the content of each of which is incorporated herein by reference in its entirety.

### SEQUENCE LISTING

This application contains a Sequence Listing that has been submitted electronically as an XML file named 61192-0004002_SL_ST26.xml. The XML file, created on Jun. 10, 2026, is 964,603 bytes in size. The material in the XML file is hereby incorporated by reference in its entirety.

### FIELD

The present disclosure relates generally to the field of antibodies and single domain antibodies binding to DLL3.

### BACKGROUND

Delta-like ligand 3 (DLL3, NCBI Gene ID: 10683, UniProt Ref: MOR177) is an inhibitory Notch pathway ligand that is highly upregulated and aberrantly expressed on the cell surface in SCLC and other high-grade neuroendocrine tumors (Sabari et al., Nat Rev Clin Oncol. 2017; 14(9):549-561; Saunders et al., Sci Transl Med. 2015; 7(302):302ra136). There is a need in the art for DLL3-specific antibodies and antigen-binding fragments thereof for use in the treatment of these and other cancers.

### SUMMARY

The present disclosure provides single domain antibodies (sdAb) comprising a complementarity determining region (CDR) 1, a CDR2, and a CDR3, wherein: the CDR1 comprises an amino acid sequence selected from SEQ ID NOs: 1, 6, 10, 14, 18, 22, 26, 30, 33, 36, 39, 46, 52, 60, 65, 69, 73, 77, and 83; the CDR2 comprises an amino acid sequence selected from SEQ ID NOs: 2, 7, 11, 15, 19, 23, 27, 40, 43, 47, 53, 61, 66, 74, 78, 81, and 82; and the CDR3 comprises an amino acid sequence selected from SEQ ID NOs: 3, 8, 12, 16, 20, 24, 28, 31, 34, 37, 41, 44, 48, 54, 62, 67, 71, 75, 79, and 84.

In some embodiments of the disclosure, the sdAb comprises a CDR1, a CDR2, and a CDR3, wherein: the CDR1 comprises an amino acid sequence of SEQ ID NO: 46; the CDR2 comprises an amino acid sequence of SEQ ID NO: 47; and the CDR3 comprises an amino acid sequence of SEQ ID NO: 48.

In some embodiments of the disclosure, the sdAb comprises a CDR1, a CDR2, and a CDR3, wherein: the CDR1 comprises an amino acid sequence of SEQ ID NO: 52; the CDR2 comprises an amino acid sequence of SEQ ID NO: 53; and the CDR3 comprises an amino acid sequence of SEQ ID NO: 54.

In some embodiments of the disclosure, the sdAb comprises a CDR1, a CDR2, and a CDR3, wherein: the CDR1 comprises an amino acid sequence of SEQ ID NO: 18; the CDR2 comprises an amino acid sequence of SEQ ID NO: 19; and the CDR3 comprises an amino acid sequence of SEQ ID NO: 20.

In some embodiments of the disclosure, the sdAb comprises a CDR1, a CDR2, and a CDR3, wherein: the CDR1 comprises an amino acid sequence of SEQ ID NO: 83; the CDR2 comprises an amino acid sequence of SEQ ID NO: 2; and the CDR3 comprises an amino acid sequence of SEQ ID NO: 84.

In some embodiments of the disclosure, the sdAb comprises a CDR1, a CDR2, and a CDR3, wherein: the CDR1 comprises an amino acid sequence of SEQ ID NO: 60; the CDR2 comprises an amino acid sequence of SEQ ID NO: 61; and the CDR3 comprises an amino acid sequence of SEQ ID NO: 62.

In some embodiments of the disclosure, the sdAb comprises a CDR1, a CDR2, and a CDR3, wherein: the CDR1 comprises an amino acid sequence of SEQ ID NO: 65; the CDR2 comprises an amino acid sequence of SEQ ID NO: 66; and the CDR3 comprises an amino acid sequence of SEQ ID NO: 67.

In some embodiments of the disclosure, the sdAb comprises a CDR1, a CDR2, and a CDR3, wherein: the CDR1 comprises an amino acid sequence of SEQ ID NO: 69; the CDR2 comprises an amino acid sequence of SEQ ID NO: 23; and the CDR3 comprises an amino acid sequence of SEQ ID NO: 24.

In some embodiments of the disclosure, the sdAb comprises a CDR1, a CDR2, and a CDR3, wherein: the CDR1 comprises an amino acid sequence of SEQ ID NO: 14; the CDR2 comprises an amino acid sequence of SEQ ID NO: 43; and the CDR3 comprises an amino acid sequence of SEQ ID NO: 71.

In some embodiments of the disclosure, the sdAb comprises a CDR1, a CDR2, and a CDR3, wherein: the CDR1 comprises an amino acid sequence of SEQ ID NO: 73; the CDR2 comprises an amino acid sequence of SEQ ID NO: 74; and the CDR3 comprises an amino acid sequence of SEQ ID NO: 75.

In some embodiments of the disclosure, the sdAb comprises an amino acid sequence that is at least 95%, at least 96%, at least 97%, at least 98%, or at least 99% identical to an amino acid sequence selected from SEQ ID NOs: 4, 5, 9, 13, 17, 21, 25, 29, 32, 35, 38, 42, 45, 49, 55, 59, 63, 64, 68, 70, 72, and 76. In some embodiments, the sdAb comprises or consists of an amino acid sequence selected from SEQ ID NOs: 4, 5, 9, 13, 17, 21, 25, 29, 32, 35, 38, 42, 45, 49, 55, 59, 63, 64, 68, 70, 72, and 76.

The present disclosure provides single domain antibodies (sdAb) comprising (a) a human framework region sequence and (b) a complementarity determining region (CDR) 1, a CDR2, and a CDR3, wherein: (i) the CDR1 comprises an amino acid sequence selected from SEQ ID NOs: 1, 6, 10, 14, 18, 22, 26, 30, 33, 36, 39, 46, 52, 60, 65, 69, 73, 77, and 83; (ii) the CDR2 comprises an amino acid sequence selected from SEQ ID NOs: 2, 7, 11, 15, 19, 23, 27, 40, 43, 47, 53, 61, 66, 74, 78, 81, and 82; and (iii) the CDR3 comprises an amino acid sequence selected from SEQ ID NOs: 3, 8, 12, 16, 20, 24, 28, 31, 34, 37, 41, 44, 48, 54, 62, 67, 71, 75, 79, and 84.

In some embodiments of the disclosure, the sdAb comprises a human framework region sequence, a CDR1, a CDR2 and a CDR3, wherein: the CDR1 comprises an amino acid sequence of SEQ ID NO: 46; the CDR2 comprises an amino acid sequence of SEQ ID NO: 47; and the CDR3 comprises an amino acid sequence of SEQ ID NO: 48.

In some embodiments of the disclosure, the sdAb comprises a human framework region sequence, a CDR1, a CDR2 and a CDR3, wherein: the CDR1 comprises an amino acid sequence of SEQ ID NO: 52; the CDR2 comprises an amino acid sequence of SEQ ID NO: 53; and the CDR3 comprises an amino acid sequence of SEQ ID NO: 54.

In some embodiments of the disclosure, the sdAb comprises a human framework region sequence, a CDR1, a CDR2 and a CDR3, wherein: the CDR1 comprises an amino acid sequence of SEQ ID NO: 18; the CDR2 comprises an amino acid sequence of SEQ ID NO: 19; and the CDR3 comprises an amino acid sequence of SEQ ID NO: 20.

In some embodiments of the disclosure, the sdAb comprises a human framework region sequence, a CDR1, a CDR2 and a CDR3, wherein: the CDR1 comprises an amino acid sequence of SEQ ID NO: 83; the CDR2 comprises an amino acid sequence of SEQ ID NO: 2; and the CDR3 comprises an amino acid sequence of SEQ ID NO: 84.

In some embodiments of the disclosure, the sdAb comprises a human framework region sequence, a CDR1, a CDR2 and a CDR3, wherein: the CDR1 comprises an amino acid sequence of SEQ ID NO: 60; the CDR2 comprises an amino acid sequence of SEQ ID NO: 61; and the CDR3 comprises an amino acid sequence of SEQ ID NO: 62.

In some embodiments of the disclosure, the sdAb comprises a human framework region sequence, a CDR1, a CDR2 and a CDR3, wherein: the CDR1 comprises an amino acid sequence of SEQ ID NO: 65; the CDR2 comprises an amino acid sequence of SEQ ID NO: 66; and the CDR3 comprises an amino acid sequence of SEQ ID NO: 67.

In some embodiments of the disclosure, the sdAb comprises a human framework region sequence, a CDR1, a CDR2 and a CDR3, wherein: the CDR1 comprises an amino acid sequence of SEQ ID NO: 69; the CDR2 comprises an amino acid sequence of SEQ ID NO: 23; and the CDR3 comprises an amino acid sequence of SEQ ID NO: 24.

In some embodiments of the disclosure, the sdAb comprises a human framework region sequence, a CDR1, a CDR2 and a CDR3, wherein: the HCDR1 comprises an amino acid sequence of SEQ ID NO: 14; the HCDR2 comprises an amino acid sequence of SEQ ID NO: 43; and the HCDR3 comprises an amino acid sequence of SEQ ID NO: 71.

In some embodiments of the disclosure, the sdAb comprises a human framework region sequence, a CDR1, a CDR2 and a CDR3, wherein: the CDR1 comprises an amino acid sequence of SEQ ID NO: 73; the CDR2 comprises an amino acid sequence of SEQ ID NO: 74; and the CDR3 comprises an amino acid sequence of SEQ ID NO: 75.

In some embodiments of the disclosure, the sdAb comprising a human framework region sequence comprises an amino acid sequence that is at least 95%, at least 96%, at least 97%, at least 98%, or at least 99% identical to an amino acid sequence selected from SEQ ID NOs: 50, 51, 56, 57 and 58. In some embodiments, the sdAb comprising a human framework region sequence comprises or consists of an amino acid sequence selected from SEQ ID NOs: 50, 51, 56, 57 and 58.

In some embodiments of the disclosure, the sdAb of the disclosure specifically binds to a Delta-like ligand 3 (DLL3) protein. In some embodiments, the DLL3 protein is a human DLL3 protein.

The disclosure provides chimeric antigen receptors (CARs) comprising an extracellular antigen binding domain, a hinge, a transmembrane domain, and a cytoplasmic domain wherein the extracellular antigen binding domain is the sdAb of the disclosure.

The disclosure provides biomolecules comprising the sdAb of the disclosure.

The disclosure provides polynucleotides encoding the single domain antibodies of the disclosure, the CARs of the disclosure, or the biomolecules of the disclosure. In some embodiments, the polynucleotide is an mRNA molecule.

The disclosure provides vectors comprising the polynucleotide of the disclosure.

The disclosure provides binding construct comprising the sdAb of the disclosure. The disclosure provides binding construct comprising the sdAb of the disclosure and an Fc domain.

The disclosure provides bispecific binding constructs comprising a first binding domain and a second binding domain, wherein the first binding domain comprises the sdAb of the disclosure; and the second binding domain is selected from an antigen binding domain that specifically binds to CD3, CD16 or NKp46, or human serum albumin (HSA), or an Fc domain.

The disclosure provides trispecific binding constructs comprising a first binding domain, a second binding domain and a third binding domain, wherein the first binding domain comprises the sdAb of the disclosure; the second binding domain comprises an Fc domain or an antigen binding domain that specifically binds to human serum albumin (HSA); and the third binding domain is an antigen binding domain that specifically binds to CD3, CD16 or NKp46. In some embodiments, the second binding domain is an antigen binding domain that specifically binds to HSA, and the third binding domain is an antigen binding domain that specifically binds to CD3.

The disclosure provides polynucleotides encoding the binding construct of the disclosure, the bispecific binding constructs of the disclosure, or the trispecific binding constructs of the disclosure. In some embodiments, the polynucleotide is an mRNA molecule.

The disclosure provides vectors comprising the polynucleotide of the disclosure.

The disclosure provides methods of treating cancer in a subject in need thereof, comprising administering the sdAb, the CAR, the biomolecule, the binding construct, the bispecific binding construct, or the trispecific binding construct of the disclosure.

The disclosure provides uses of the sdAb, the CAR, the biomolecule, the binding construct, the bispecific binding construct, or the trispecific binding construct of the disclosure, for treating cancer in a subject in need thereof.

The disclosure provides uses of the sdAb, the CAR, the biomolecule, the binding construct, the bispecific binding construct, or the trispecific binding construct of the disclosure in the manufacture of a medicament for treating cancer in a subject in need thereof.

In some embodiments of the disclosure, the cancer is selected from non-small cell lung cancer (NSCLC), small cell lung cancer (SCLC), small cell bladder cancer, large cell neuroendocrine carcinoma (LCNEC), castration-resistant small cell neuroendocrine prostate cancer (CRPC-NE), carcinoid (e.g., pulmonary carcinoid), glioblastoma multiforme-IDH mutant (GBM-IDH mutant), Merkel cell carcinoma, and Gastric neuroendocrine cancer.

In some embodiments, the sdAb or the binding construct of the disclosure is administered in combination with an oncolytic virus or a polynucleotide encoding the oncolytic virus. In some embodiments, the sdAb or the binding construct, and the oncolytic virus or the polynucleotide encoding the oncolytic virus, are administered concurrently. In some embodiments, the sdAb or the binding construct, and the oncolytic virus or the polynucleotide encoding the oncolytic virus, are administered sequentially. In some embodiments, the oncolytic virus is a picornavirus. In some embodiments, the picornavirus is a Coxsackievirus. In some embodiments, the picornavirus is a Seneca Valley virus. In some embodiments, the polynucleotide encoding the oncolytic virus is encapsulated in a lipid nanoparticle.

The disclosure provides oncolytic viruses expressing one or more payload molecules, wherein the one or more payload molecules comprise the sdAb, the binding construct, the bispecific binding construct, or the trispecific binding construct of the disclosure. In some embodiments, the oncolytic virus is selected from herpes simplex virus, an adenovirus, a polio virus, a vaccinia virus, a measles virus, a vesicular stomatitis virus, an orthomyxovirus, a parvovirus, a maraba virus, a picornavirus, a togaviriadae virus, a semliki forest virus, a sindbis virus, a paramyxoviridae virus, and a sendai virus. In some embodiments, the picornavirus is a coxsackievirus or a seneca valley virus.

The disclosure provides methods of treating cancer in a subject in need thereof, comprising administering the oncolytic virus of the disclosure to the subject.

The disclosure provides uses of the oncolytic virus of the disclosure for treating cancer in a subject in need thereof.

The disclosure provides uses of the oncolytic virus of the disclosure in the manufacture of a medicament for treating cancer in a subject in need thereof.

The disclosure provides messenger RNA (mRNA) polynucleotides encoding the sdAb, the binding construct, the bispecific binding construct or the trispecific binding construct of the disclosure.

The disclosure provides methods of treating cancer in a subject in need thereof, comprising administering the mRNA polynucleotide of the disclosure to the subject.

The disclosure provides uses of the mRNA polynucleotide of the disclosure for treating cancer in a subject in need thereof.

The disclosure provides uses of the mRNA polynucleotide of the disclosure in the manufacture of a medicament for treating cancer in a subject in need thereof. In some embodiments of the disclosure, the mRNA polynucleotide is administered in combination with an oncolytic virus. In some embodiments, the mRNA polynucleotide is administered in combination with a polynucleotide encoding an oncolytic virus.

### BRIEF DESCRIPTION OF THE DRAWINGS

FIG. 1 illustrates the cytotoxicity of multiple anti-DLL3/anti-CD3 bispecific binding constructs.

FIG. 2 illustrates the species cross-reactivity of two different anti-DLL3/anti-CD3 bispecific binding constructs.

FIG. 3 shows the binding properties of DLL3 binding constructs determined by binding assay, ELISA, and cell studies.

FIG. 4A illustrates the species cross-reactivity of the anti-DLL3/anti-CD3/anti-HSA trispecific binding construct on Cyno-DLL3-CHO cells and human-DLL3-CHO cells measured by flowcytometry. FIG. 4B illustrates the species cross-reactivity of the anti-DLL3/anti-CD3/anti-HSA trispecific binding construct on human and Cyno CD8 T cells measured by flow cytometry.

FIG. 5A illustrates the expression of DLL3 on SHP-77, NCI-H82 and NCI-H1299 cells transduced with human DLL3. The percent of cells expressing DLL3 and the mean fluorescence intensity (MFI) within the positive cells were measured by flow cytometry. FIG. 5B illustrates the binding of the anti-DLL3/anti-CD3/anti-HSA trispecific binding construct on the NSCLC cell line NCI-H1299, or NCI-H1299 cells transduced with human DLL3, as measured by flow cytometry. FIG. 5C illustrates the binding of the anti-DLL3/anti-CD3/anti-HSA trispecific binding construct on the NSCLC cell lines NCI-H82 and SHP-77, as measured by flow cytometry.

FIG. 6A illustrates the cytotoxicity of the anti-DLL3/anti-CD3/anti-HSA trispecific binding construct on Cyno-DLL3-expressing CHO cells and human-DLL3-expressing CHO cells by cyno or human T cells, respectively. FIG. 6B illustrates the cytotoxicity of the anti-DLL3/anti-CD3/anti-HSA trispecific binding construct on the NSCLC cell line NCI-H1299 transduced with human DLL3 by human T cells. FIG. 6C illustrates the cytotoxicity of the anti-DLL3/anti-CD3/anti-HSA trispecific binding construct on NSCLC cell line NCI-H82 and SHP-77 by human T cells.

FIG. 7 illustrates the pharmacokinetics (PK) of the anti-DLL3/anti-CD3/anti-HSA trispecific binding construct compared to the anti-DLL3/anti-CD3 bispecific construct in mouse.

FIG. 8A illustrates the in vivo efficacy of the anti-DLL3/anti-CD3/anti-HSA trispecific binding construct and/or LNP containing synthetic SVV RNA viral genome in an NCI-H82 tumor model using humanized-NSG mice. FIG. 8B illustrates the in vivo efficacy of the anti-DLL3/anti-CD3/anti-HSA trispecific binding construct and/or LNP containing synthetic SVV RNA viral genome in an NCI-H1299-hDLL3 tumor model using humanized-NSG mice.

FIG. 9 illustrates the in vivo efficacy of the anti-DLL3/anti-CD3 bispecific binding construct and/or LNP containing synthetic SVV RNA viral genome in an NCI-H1299-hDLL3 tumor model using humanized-NSG mice.

FIG. 10 illustrates a trispecific binding construct of the present disclosure.

### DETAILED DESCRIPTION

The details of the disclosure are set forth in the accompanying description below. Although methods and materials similar or equivalent to those described herein can be used in the practice or testing of the present disclosure, illustrative methods, and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide.

The term “binding construct” refers to a molecule comprising at least one binding domain. A “binding domain” is a domain of a polypeptide that binds to a cognate antigen (e.g., an antigen binding domain such as an antibody or antigen binding fragment thereof), receptor (e.g., an Fc receptor), or other ligand. In some embodiments, the binding constructs described herein comprise at least one, two, or three binding domains. In some embodiments, at least one of the binding domains is an antigen binding domain. Optionally, a binding construct may comprise a non-polypeptide moiety (a non-limiting example is an antibody-drug conjugate).

The terms “DLL3 binding construct” or “anti-DLL3 construct” are used interchangeably and refer to a binding construct comprising a DLL3 binding domain.

The term “bispecific binding construct” refers to a binding construct that comprises two binding domains that each bind to different antigens or targets.

“DVD-Ig” is a dual variable domain immunoglobulin protein, which contains an Fc region and constant regions in a configuration similar to a conventional IgG, while each arm of the molecule comprises two heavy chain variable domains and two light chain variable domains (unlike conventional IgG which only contains one heavy chain variable domain and one light chain variable domain in each arm). The two heavy chain variable domains within an arm are linked in tandem and may possess different binding specificities, and so is the two light chain variable domains.

“mAb2” is a full-length antibody having an overall structure similar to those of regular IgG, except that the Fc region comprises a second distinct antigen binding site which allows mAb2 to bind to two different antigens at the same time.

Fabs-in-tandem immunoglobulins (FIT-Ig) contains an Fc region in a configuration similar to a conventional IgG but each arm of the molecule comprises two or more different Fab domains linked in tandem and each Fab specifically binds to a distinct antigen. In some embodiments, the C-terminus of the constant domain of the light chain of the first Fab domain is linked to the N-terminus of the variable domain of the heavy chain of the second Fab domain.

The term “knobs-in-holes” or “KIH” refers to a format of antibody structure comprising amino acid mutations in the CH3 domain of the heavy chain that result in preferential formation of heterodimeric heavy chains. In some embodiments, the knob is a tyrosine residue and the hole is a threonine residue. In some embodiments, the CH3 domain of the first heavy chain comprises a mutation equivalent to T22Y of SEQ ID NO: 112, and the CH3 domain of the second heavy chain comprises a mutation equivalent to Y63T of SEQ ID NO: 112. Here, the polypeptide sequence of the reference CH3 domain comprises EPQVYTLPPSRDELTKNQVS LTCLVKGFYPSDIAVEWESNGQPENNYKTTPPVLDSDGSFFLYSKLTVDKSRWQQGNVF SCSVMHEALHNHYTQKSLSLSP (SEQ ID NO: 112). As a result, the first heavy chain and the second heavy chain preferentially pair with each other during the formation of antibodies.

The term “BiTE”, when referring to a class of antibody or antibody-like molecules, refers to bispecific T-cell engagers. Such molecules have a first binding domain that is specific for an antigen associated with a diseased state (e.g., an antigen expressed on cancerous cells) and second binding domain that is specific for an antigen associated with T cells. In some embodiments, BiTEs are fusion proteins comprising two single-chain variable fragments (scFvs) of different antibodies on a single peptide chain of about 55 kilodaltons. One of the scFvs binds to T-cells via the CD3 receptor, and the other to a tumor cell via a tumor specific molecule. In some embodiments, one or more of the scFv is replaced by VHH. In some embodiments, one of the scFv or VHH specifically binds DLL3.

The term “trispecific binding construct” refers to binding constructs that comprise three binding domains each of which specifically bind to a different antigen or target. In some embodiments, a trispecific binding construct of the disclosure may be a trispecific antibody of the disclosure. In some embodiments, a trispecific binding construct of the disclosure may comprise a first antigen binding domain, a second antigen binding domain, and a third antigen binding domain. In some embodiments, the first antigen, the second antigen and the third antigen are all different.

The term “antigen-binding fragment” as used herein refers to a polypeptide fragment that contains at least one Complementarity-determining region (CDR) of an immunoglobulin heavy and/or light chain that specifically binds to at least one epitope of the antigen of interest. In this regard, an antigen-binding fragment may comprise 1, 2, 3, 4, 5, or all 6 CDRs of a variable heavy chain (VH) and variable light chain (VL) sequence. Antigen-binding fragments include proteins that comprise a portion of a full length antibody, generally the antigen binding or variable region thereof, such as Fab, F(ab′)2, Fab′, Fv fragments, minibodies, diabodies, single domain antibody (sdAb), single-chain variable fragments (scFv), multispecific antibodies formed from antibody fragments, and any other modified configuration of the immunoglobulin molecule that comprises an antigen-binding site or fragment of the required specificity. In certain embodiments of the disclosure, an antigen-binding fragment, rather than an intact antibody, is used to increase tissue penetration or tumor penetration. In other embodiments, antigen-binding fragments are further modified to increase serum half-life.

The term “half-life”, when referring to an antibody and antigen-binding fragment thereof in vivo, refers to a pharmacokinetic property of a molecule that is a measure of the mean survival time of the molecules in vivo following their administration. Half-life can be expressed as the time required to eliminate fifty percent (50%) of a known quantity of the molecule from the subject's body (e.g., human patient or other mammal) or a specific compartment thereof, for example, as measured in serum, i.e., circulating half-life, or in other tissues. In general, an increase in half-life results in an increase in mean residence time (MRT) in circulation for the molecule administered.

The term “F(ab)” refers to two of the protein fragments resulting from proteolytic cleavage of IgG molecules by the enzyme papain. Each F(ab) comprises a covalent heterodimer of the VH chain and VL chain and includes an intact antigen-binding site. Each F(ab) is a monovalent antigen-binding fragment. The term “Fab′” refers to a fragment derived from F(ab′)2 and may contain a small portion of Fc. Each Fab′ fragment is a monovalent antigen-binding fragment.

The term “F(ab′)2” refers to a protein fragment of IgG generated by proteolytic cleavage by the enzyme pepsin. Each F(ab′)2 fragment comprises two F(ab′) fragments and is therefore a bivalent antigen-binding fragment.

An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096. In some embodiments, the Fv fragment can be produced by preferential proteolytic cleavage of an IgM, and on rare occasions of an IgG or IgA immunoglobulin molecule. Fv fragments are, however, more commonly derived using recombinant techniques known in the art.

Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The term “diabody” refers to a bispecific antibody in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)).

The term “nanobody” or a “single domain antibody” refers to an antigen-binding fragment consisting of a single monomeric variable antibody domain. The Nanoclone method is a method for generating Nanobodies against a desired target based on automated high-throughput selection of B-cells. (See, WO 2006/079372)

The term “variable new antigen receptor” (VNAR) refers to the variable domain of immunoglobulin new antigen receptor (IgNAR), which contains only two complementarity-determining regions (CDRs)—CDR1 and CDR3 (see Cheong et al., Int J Biol Macromol. 2020 Mar. 15; 147:369-375).

The term “monoclonal antibody” as used herein refers to an antibody obtained from a population of substantially homogeneous antibodies, i.e., the individual antibodies comprising the population are identical except for possible naturally occurring mutations that may be present in minor amounts.

In some embodiments, the term “chimeric antibody” as used herein refers to a monoclonal antibody in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity.

The term “single chain variable fragment” or “scFv” refers to a fusion protein of the variable regions of the heavy (VH) and light chains (VL) of immunoglobulins, connected with a short linker peptide of ten to about 25 amino acids. Huston et al. (1988) Proc. Nat. Acad. Sci. USA 85(16):5879-5883. The linker can connect the N-terminus of the VH with the C-terminus of the VL, or vice versa. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

The term “antigen” refers to a molecule or a portion of a molecule capable of being bound by an antibody or an antigen-binding fragment thereof and additionally capable of being used in an animal to produce antibodies capable of binding to an epitope of that antigen. An antigen may have one or more epitopes. Embodiments herein contemplate the use of a DLL3 protein, or a DLL3 protein conjugated to a hapten, as an antigen.

The term “epitope” refers to a region of an antigen that is bound by an antibody. Epitope determinants can include chemically active surface groupings of molecules such as amino acids, sugar side chains, phosphoryl, or sulfonyl and may have specific three-dimensional structural characteristics, and/or specific charge characteristics.

Herein, the term “specifically binds” refers to the ability of an antibody or antigen-binding fragment thereof to bind a target antigen with a binding affinity (Ka) of at least 10⁵ M⁻¹ while not significantly binding other components or antigens present in a mixture. Reference to an anti-DLL3 antibody herein refers to an antibody or antigen-binding fragment thereof that specifically binds to DLL3.

Binding affinity (Ka) refers to an equilibrium association of a particular interaction expressed in the units of 1/M or M⁻¹. Antibodies or antigen-binding fragments thereof can be classified as “high affinity” antibodies or antigen-binding fragments thereof and “low affinity” antibodies or antigen-binding fragments thereof. “High affinity” antibodies or antigen-binding fragments thereof refer to those antibodies or antigen-binding fragments thereof with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” antibodies or antigen-binding fragments thereof refer to those antibodies or antigen-binding fragments thereof with a Ka of up to 10⁷ M⁻¹, up to 10⁶ M⁻¹, or up to 10⁵ M⁻¹. Alternatively, affinity can be defined as an equilibrium dissociation constant (K_{D}) of a particular binding interaction with units of M (e.g., 10⁻⁵ M to 10⁻¹³, or about 500 nM, about 300 nM, about 250 nM, about 200 nM, about 150 nM, about 100 nM, about 50 nM, about 25 nM, about 10 nM, about 5 nM, about 2 nM, about 1 nM, about 500 pM, about 200 pM, about 100 pM, about 50 pM, about 20 pM, about 10 pM, about 5 pM, about 2 pM, about 1 pM, about 0.5 pM, about 0.2 pM, or about 0.1 pM, including all ranges and subranges therebetween). Affinities of binding domain polypeptides and single chain polypeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

A “conservative substitution” is recognized in the art as a substitution of one amino acid for another amino acid that has similar properties. Exemplary conservative substitutions are well-known in the art (see, e.g., PCT Application Publication No. WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8).

As used herein, the term “derivative” refers to a modification of one or more amino acid residues of a peptide by chemical or biological means, either with or without an enzyme, e.g., by glycosylation, alkylation, acylation, ester formation, or amide formation.

As used herein, a polypeptide or polynucleotide from which another polypeptide or polynucleotide is derived from is referred to as the “parental” or “reference” polynucleotide or polypeptide. For example, a humanized antibody can be derived from a parental murine antibody.

The term “variant” or “variants” as used herein refers to a polynucleotide or polypeptide with a sequence differing from that of a reference polynucleotide or polypeptide but retaining essential properties of the parental polynucleotide or polypeptide. Generally, variant polynucleotide or polypeptide sequences are overall closely similar, and, in many regions, identical to the parental polynucleotide or polypeptide. For instance, a variant polynucleotide or polypeptide may exhibit at least 70%, at least 80%, at least 90%, at least 91%, at least 92%, at least 93%, at least 94%, at least 95%, at least 96%, at least 97%, at least 98% at least 99%, or at least 99.5% sequence identity compared to the parental polynucleotide or polypeptide.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

The term “substantially identical” refers to a polypeptide sequence that contains a sufficient number of identical amino acids to a second polypeptide sequence such that the first and second polypeptide sequence have similar activity. Polypeptides that are substantially identical are at least 80%, at least 85%, at least 90%, at least 95%, at least 96%, at least 97%, at least 98%, at least 99%, or 100% identical in amino acid sequence.

“Fc region” or “Fc domain” refers to a polypeptide sequence corresponding to or derived from the portion of an antibody that is capable of binding to Fc receptors on cells and/or the C1q component of complement, thereby mediating the effector function of an antibody. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc region is a homodimeric protein comprising two polypeptides that are associated by disulfide bonds, and each comprising a hinge region, a CH2 domain, and a CH3 domain. However, more recently the term has been applied to the single chain monomer component consisting of CH3, CH2, and at least a portion of the hinge sufficient to form a disulfide-linked dimer with a second such chain. As such, and depending on the context, use of the terms “Fc region” or “Fc domain” will refer herein to either the dimeric form or the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc domain includes variants of naturally occurring sequences.

The term “immunoglobulin constant region” or “constant region” refers to a peptide or polypeptide sequence that corresponds to or is derived from part or all of one or more constant domains of an immunoglobulin (e.g., CH1, CH2, CH3). In certain embodiments, the constant region does not comprise a CH1 domain. In certain embodiments, the constant domains making up the constant region are human

The terms “light chain variable region” (also referred to as “light chain variable domain” or “VL”) and “heavy chain variable region” (also referred to as “heavy chain variable domain” or “VH”) refer to the variable binding region from an antibody light and heavy chain, respectively. The variable binding regions are made up of discrete, well-defined sub-regions known as “complementarity determining regions” (CDRs) and “framework regions” (FRs).

The term “immunoglobulin light chain constant region” (also referred to as “light chain constant region” or “CL”) is a constant region from an antibody light chain.

The term “immunoglobulin heavy chain constant region” (also referred to as “heavy chain constant region” or “CH”) refers to the constant region from the antibody heavy chain. The CH is further divisible, depending on the antibody isotype into CH1, CH2, and CH3 (IgA, IgD, IgG), or CH1, CH2, CH3, and CH4 domains (IgE, IgM).

As used herein, the term “complementarity determining region” or “CDR” refer to an immunoglobulin (antibody) molecule. There are three CDRs per variable domain: CDR1, CDR2 and CDR3 in the variable domain of the light chain and CDR1, CDR2 and CDR3 in the variable domain of the heavy chain.

In some embodiments, a “hinge” or a “hinge region” refers to a polypeptide derived from an immunoglobulin hinge region and located between an antigen-binding domain (e.g., a DLL3 binding domain) and an immunoglobulin constant region in a polypeptide described herein. A “wild-type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and can comprise a human IgG hinge region (e.g., and IgG1, IgG2, IgG3, or IgG4 hinge region).

An “altered immunoglobulin hinge region” or “variant immunoglobulin hinge region” refers to a hinge region polypeptide with one or more mutations, substitutions, insertions, or deletions compared to a corresponding parental wild-type immunoglobulin hinge region. Typically, an altered immunoglobulin hinge region that is a fragment of a wild type immunoglobulin hinge region comprises an IgG core hinge region (e.g., a polypeptide comprising the sequence C-X-X-C(SEQ ID NO: 761), wherein X is any amino acid) as disclosed in U.S. Patent Application Publication Nos. 2013/0129723 and 2013/0095097.

As used herein, the term “humanized” refers to an antibody or antigen-binding fragment thereof derived from a non-human species that retains the antigen-binding properties of the original non-human antibody. In some embodiments, the binding fragments of an antibody (e.g., light and heavy chain variable regions, Fab, scFv) are humanized. Non-human antigen-binding fragments can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf BW, Dalton BJ, eds. Cellular adhesion: molecular definition to therapeutic potential. New York: Plenum Press, 1994: 291-312). If derived from a non-human source, other regions of the antibody, such as the hinge region and constant region domains, can also be humanized.

The term “human framework region sequence” refers to the amino acid sequence of all the framework regions (i.e., non-CDR regions) of a variable domain that is derived from a human germline sequence. Typically, a human framework region sequence comprises four framework regions (FRs): FR1, FR2, FR3 and FR4, which are separated by CDRs. The organization of these regions is, from N-terminus to C-terminus, N′-FR1-CDR1-FR2-CDR2-FR3-CDR3-FR4-C′. The human framework region sequence may comprise one or more amino acid substitutions, insertions and/or deletions in the framework of the variable domain as compared to the corresponding human germline sequence. The substitution may be a conservative substitution. Such mutations can be readily ascertained by comparing the amino acid sequences disclosed herein to germline sequences available from, for example, public antibody sequence databases. For example, the human framework region sequence may comprises 10 or fewer, 9 or fewer, 8 or fewer, 7 or fewer, 6 or fewer, 5 or fewer, 4 or fewer, 3 or fewer, 2 or fewer, or 1 mutation as compared to the closest germline sequence found in public antibody sequence databases.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The term “polynucleotide” as referred to herein means single-stranded or double-stranded nucleic acid polymers. In certain embodiments, the nucleotides comprising the polynucleotide can be RNA or DNA or a modified form of either type of nucleotide, such as a modified messenger RNA. Said modifications may include, but are not limited to, base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

As used herein, a “polypeptide” or “protein” refers to a single, linear, and contiguous arrangement of covalently linked amino acids. Polypeptides can form one or more intrachain disulfide bonds. The terms polypeptide and protein also encompass embodiments where two polypeptide chains link together in a non-linear fashion, such as via an interchain disulfide bond. Herein, a protein or polypeptide may be an antibody or an antigen-binding fragment of an antibody.

As used herein, the terms “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual or prevent onset of additional associated diseases.

As used in this specification and the appended claims, the singular forms “a,” “an” and “the” include plural references unless the content clearly dictates otherwise.

As used in this specification, the term “and/or” is used in this disclosure to either “and” or “or” unless indicated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

### Anti-DLL3 Binding Constructs

The present disclosure provides binding constructs and biomolecules that specifically bind to Delta-like ligand 3 (DLL3). In some embodiments, the binding construct is a single domain antibody (sdAb). In some embodiments, the sdAb is a variable domain heavy chain (VHH) sdAb. In some embodiments, the sdAb is a variable new antigen receptor (VNAR) sdAb. VNAR sdAbs are described in Cheong et al., Int J Biol Macromol. 2020 Mar. 15; 147:369-375, the content of which is incorporated by reference in its entirety.

The term “biomolecule” refers to a biological molecule (e.g., a small molecule, a protein, a nucleic acid, and the like). In some embodiments, the present disclosure provides biomolecules comprising a DLL3 binding domain. Such biomolecules include DLL3 binding constructs (antibodies, sdAbs, bispecific, and trispecific binding constructs), antibody-drug conjugates comprising a DLL3 binding domain, and engineered antigen receptors (e.g., chimeric antigen receptors and engineered TCRs) that specifically bind to DLL3.

In some embodiments, the binding constructs of the disclosure comprises at least one antigen binding domain that specifically or selectively binds an epitope, an antigen or a target comprised of or derived from a DLL3 polypeptide. In some embodiments, the DLL3 polypeptide is a mammalian DLL3 polypeptide. In some embodiments, the DLL3 polypeptide is a murine DLL3 polypeptide (See e.g., UniProt Ref #O88516). In some embodiments, the DLL3 polypeptide is a primate DLL3 polypeptide. In some embodiments, the DLL3 polypeptide is a human DLL3 polypeptide (See e.g., UniProt Ref #Q9NYJ7).

Delta-like ligand 3 (DLL3, NCBI Gene ID: 10683, UniProt Ref: Q9NYJ7) is an inhibitory Notch pathway ligand that is highly upregulated and aberrantly expressed on the cell surface in SCLC and other high-grade neuroendocrine tumors (Sabari et al., Nat Rev Clin Oncol. 2017; 14(9):549-561; Saunders et al., Sci Transl Med. 2015; 7(302):302ra136). Notch signaling is downregulated during neuroendocrine tumor growth and is inhibited by DLL3 expression (Kunnimalaiyaan et al., Oncologist. 2007; 12(5):535-542. doi: 10.1634/theoncologist.12-5-535; Lehman et al., Curr Oncol Rep. 2017; 19(7):49). DLL3 expression is regulated by achaete-scute homolog 1 (ASCL1), a transcription factor that is required for proper development of pulmonary neuroendocrine cells and is an oncogenic driver in SCLC (Augustyn et al., Proc Natl Acad Sci USA. 2014; 111(41):14788-14793; Borromeo et al., Cell Rep. 2016; 16(5):1259-1272). In preclinical models, DLL3 expression promotes SCLC migration and invasion through a mechanism that involves control of the epithelial-mesenchymal transition protein Snail (Furuta et al., Cancer Sci. 2019; 110(5):1599-1608).

In some embodiments, the present disclosure provides an anti-DLL3 antibody comprising a heavy chain variable region (VH) and light chain variable region (VL), wherein the VH comprises a heavy chain complementarity determining region (CDR) 1 (HCDR1), an HCDR2, and an HCDR3 selected from those listed in Table 1, and wherein the VL comprises a light chain CDR1 (LCDR1), an LCDR2, and an LCDR3.

In some embodiments, the present disclosure provides an anti-DLL3 antibody comprising a heavy chain variable region (VH) and light chain variable region (VL), wherein the VH comprises an HCDR1 of SEQ ID NO: 77 (GYTFTDYA), an HCDR2 of SEQ ID NO: 78 (INTYTGKP), and an HCDR3 of SEQ ID NO: 79 (SRERGYYDYSRSD), and wherein the VL comprises an LCDR1 of SEQ ID NO: 757 (KSSQSLLDSEDQKDYLG), an LCDR2 of SEQ ID NO: 758 (WATNRHT), and an LCDR3 of SEQ ID NO: 759 (EQYFAYPYT). In some embodiments, the anti-DLL3 antibody comprises a heavy chain variable region (VH) that is at least 95%, 96%, 97%, 98%, 99%, or 100% identical to SEQ ID NO: 80 and a light chain variable region (VL) that is at least 95%, 96%, 97%, 98%, 99%, or 100% identical to SEQ ID NO: 760 (DIAIIQSPSSVAVSVGETVTLSCKSSQSLLDSEDQKDYLGWYQQKPGQTPRPLIYWATNRHTGV PDRFTGSGSGTDFTLIISSVQAEDLADYYCEQYFAYPYTFGAGTKLELK).

In some embodiments, the present disclosure provides single-domain antibodies (sdAbs) that specifically bind to DLL3 (anti-DLL3 sdAbs). In some embodiments, the anti-DLL3 sdAbs described herein are VHH sdAbs or VNAR sdAbs. In some embodiments, the anti-DLL3 sdAbs described herein are VHH sdAbs. In some embodiments, the present disclosure provides a VHH sdAbs that specifically binds to DLL3 and comprises a CDR1, a CDR2, and a CDR3, wherein the CDR1 comprises an amino acid sequence selected from SEQ ID NOs: 1, 6, 10, 14, 18, 22, 26, 30, 33, 36, 39, 46, 52, 60, 65, 69, 73, 77, and 83, the CDR2 comprises an amino acid sequence selected from SEQ ID NOs: 2, 7, 11, 15, 19, 23, 27, 40, 43, 47, 53, 61, 66, 74, 78, 81, and 82, and the CDR3 comprises an amino acid sequence selected from SEQ ID NOs: 3, 8, 12, 16, 20, 24, 28, 31, 34, 37, 41, 44, 48, 54, 62, 67, 71, 75, 79, and 84. CDRs of exemplary anti-DLL3 VHH sdAbs are shown in Table 1 below.

TABLE 1
Exemplary anti-DLL3 CDR sequences

| Construct | | | SEQ |
|---|---|---|---|
| ID | Fragment | Sequence | ID |
| 2DLT10 | VH-CDR1 | GSIVGDYA | 1 |
| | VH-CDR2 | IGSEGSR | 2 |
| | VH-CDR3 | FLYNSGEDY | 3 |
| 1DLT176 | VH-CDR1 | GSIVGDYA | 1 |
| | VH-CDR2 | IGSEGSR | 2 |
| | VH-CDR3 | FLYNSGEDY | 3 |
| 2HCE138 | VH-CDR1 | GFTTDDYG | 6 |
| | VH-CDR2 | ITTGGST | 7 |
| | VH-CDR3 | NAVCSGSGCYEVSWESYDY | 8 |
| 3HCE7 | VH-CDR1 | GRTYSNYF | 10 |
| | VH-CDR2 | VSWSGDRT | 11 |
| | VH-CDR3 | AAGPLINRINY | 12 |
| 2HCE151 | VH-CDR1 | GFTFSSYA | 14 |
| | VH-CDR2 | IDSGGGST | 15 |
| | VH-CDR3 | AKEPWWADY | 16 |
| 3HCE4 | VH-CDR1 | GRAGSSYD | 18 |
| | VH-CDR2 | ITWSGNT | 19 |
| | VH-CDR3 | AAALSEKKYEY | 20 |
| 3HCE80 | VH-CDR1 | GSISSIIS | 22 |
| | VH-CDR2 | AITSGGST | 23 |
| | VH-CDR3 | NAHVRDYSGSAYY | 24 |
| 3HCE86 | VH-CDR1 | GGTFSNYD | 26 |
| | VH-CDR2 | VNRYGDYS | 27 |
| | VH-CDR3 | AARLWNSAKYAY | 28 |
| 3DLT81 | VH-CDR1 | GSIFSIYA | 30 |
| | VH-CDR2 | IGSEGSR | 2 |
| | VH-CDR3 | YAGRPPSASYSGAHY | 31 |
| 3HCE38 | VH-CDR1 | GFTFDDVA | 33 |
| | VH-CDR2 | ITSGGST | 81 |
| | VH-CDR3 | AARRDSRGQYHD | 34 |
| 2HCE6 | VH-CDR1 | GFTFSNSP | 36 |
| | VH-CDR2 | ISSNGRNT | 82 |
| | VH-CDR3 | AKGATPSVLYDY | 37 |
| 1DLT39 | VH-CDR1 | GRIFSMFP | 39 |
| | VH-CDR2 | ITTGGRP | 40 |
| | VH-CDR3 | NAVSTPELAYPYDY | 41 |
| 3HCE25 | VH-CDR1 | GFTFSSYA | 14 |
| | VH-CDR2 | INSGGGST | 43 |
| | VH-CDR3 | AKEPWVTQGS | 44 |
| 2HCE27 | VH-CDR1 | GRTFRSYA | 46 |
| | VH-CDR2 | IIMSDGST | 47 |
| | VH-CDR3 | AARRDYFTGVYDY | 48 |
| 3HCE56 | VH-CDR1 | GRTFSVDA | 52 |
| | VH-CDR2 | IDWTGGST | 53 |
| | VH-CDR3 | AARERSRTAYDY | 54 |
| 2DLT2 | VH-CDR1 | GSIVGNYA | 83 |
| | VH-CDR2 | IGSEGSR | 2 |
| | VH-CDR3 | FLYNSGDDY | 84 |
| 3HCE44 | VH-CDR1 | GSIFSINS | 60 |
| | VH-CDR2 | KASDGST | 61 |
| | VH-CDR3 | FLYANNIPY | 62 |
| 2HCE174 | VH-CDR1 | GSIFSINS | 60 |
| | VH-CDR2 | KASDGST | 61 |
| | VH-CDR3 | FLYANNIPY | 62 |
| 2HCE167 | VH-CDR1 | GRTFSSYA | 65 |
| | VH-CDR2 | ISGSGYSA | 66 |
| | VH-CDR3 | AARNERGASSSYDY | 67 |
| 3HCE18 | VH-CDR1 | GSIFSIIT | 69 |
| | VH-CDR2 | AITSGGST | 23 |
| | VH-CDR3 | NAHVRDYSGSAYY | 24 |
| 2HCE117 | VH-CDR1 | GFTFSSYA | 14 |
| | VH-CDR2 | INSGGGST | 43 |
| | VH-CDR3 | ATPFEIGS | 71 |
| 3HCE87 | VH-CDR1 | GFIFDDYI | 73 |
| | VH-CDR2 | ISWSGSAT | 74 |
| | VH-CDR3 | AASSRGPYNSGSSYDY | 75 |
| Anti- | VH-CDR1 | GYTFTDYA | 77 |
| DLL3-1 | VH-CDR2 | INTYTGKP | 78 |
| | VH-CDR3 | SRERGYYDYSRSD | 79 |

In some embodiments, the present disclosure provides a VHH sdAb that specifically binds to DLL3 and comprises a CDR1 of SEQ ID NO: 1, a CDR2 of SEQ ID NO: 2, and a CDR3 of SEQ ID NO: 3. In some embodiments, the present disclosure provides a VHH sdAb that specifically binds to DLL3 and comprises a CDR1 of SEQ ID NO: 6, a CDR2 of SEQ ID NO: 7, and a CDR3 of SEQ ID NO: 8. In some embodiments, the present disclosure provides a VHH sdAb that specifically binds to DLL3 and comprises a CDR1 of SEQ ID NO: 10, a CDR2 of SEQ ID NO: 11, and a CDR3 of SEQ ID NO: 12. In some embodiments, the present disclosure provides a VHH sdAb that specifically binds to DLL3 and comprises a CDR1 of SEQ ID NO: 14, a CDR2 of SEQ ID NO: 15, and a CDR3 of SEQ ID NO: 16.

In some embodiments, the present disclosure provides a VHH sdAb that specifically binds to DLL3 and comprises a CDR1 of SEQ ID NO: 22, a CDR2 of SEQ ID NO: 23, and a CDR3 of SEQ ID NO: 24. In some embodiments, the present disclosure provides a VHH sdAb that specifically binds to DLL3 and comprises a CDR1 of SEQ ID NO: 26, a CDR2 of SEQ ID NO: 27, and a CDR3 of SEQ ID NO: 28. In some embodiments, the present disclosure provides a VHH sdAb that specifically binds to DLL3 and comprises a CDR1 of SEQ ID NO: 30, a CDR2 of SEQ ID NO: 2, and a CDR3 of SEQ ID NO: 31. In some embodiments, the present disclosure provides a VHH sdAb that specifically binds to DLL3 and comprises a CDR1 of SEQ ID NO: 33, a CDR2 of SEQ ID NO: 81, and a CDR3 of SEQ ID NO: 34.

In some embodiments, the present disclosure provides a VHH sdAb that specifically binds to DLL3 and comprises a CDR1 of SEQ ID NO: 36, a CDR2 of SEQ ID NO: 82, and a CDR3 of SEQ ID NO: 37. In some embodiments, the present disclosure provides a VHH sdAb that specifically binds to DLL3 and comprises a CDR1 of SEQ ID NO: 39, a CDR2 of SEQ ID NO: 40, and a CDR3 of SEQ ID NO: 41. In some embodiments, the present disclosure provides a VHH sdAb that specifically binds to DLL3 and comprises a CDR1 of SEQ ID NO: 14, a CDR2 of SEQ ID NO: 43, and a CDR3 of SEQ ID NO: 44. In some embodiments, the present disclosure provides a VHH sdAb that specifically binds to DLL3 and comprises a CDR1 of SEQ ID NO: 83, a CDR2 of SEQ ID NO: 2, and a CDR3 of SEQ ID NO: 84.

In some embodiments, the present disclosure provides a VHH sdAb that specifically binds to DLL3 and comprises a CDR1 of SEQ ID NO: 18, a CDR2 of SEQ ID NO: 19, and a CDR3 of SEQ ID NO: 20. In some embodiments, the present disclosure provides a VHH sdAb that specifically binds to DLL3 and comprises a CDR1 of SEQ ID NO: 60, a CDR2 of SEQ ID NO: 61, and a CDR3 of SEQ ID NO: 62. In some embodiments, the present disclosure provides a VHH sdAb that specifically binds to DLL3 and comprises a CDR1 of SEQ ID NO: 65, a CDR2 of SEQ ID NO: 66, and a CDR3 of SEQ ID NO: 67. In some embodiments, the present disclosure provides a VHH sdAb that specifically binds to DLL3 and comprises a CDR1 of SEQ ID NO: 69, a CDR2 of SEQ ID NO: 23, and a CDR3 of SEQ ID NO: 24.

In some embodiments, the present disclosure provides a VHH sdAb that specifically binds to DLL3 and comprises a CDR1 of SEQ ID NO: 14, a CDR2 of SEQ ID NO: 43, and a CDR3 of SEQ ID NO: 71. In some embodiments, the present disclosure provides a VHH sdAb that specifically binds to DLL3 and comprises a CDR1 of SEQ ID NO: 73, a CDR2 of SEQ ID NO: 74, and a CDR3 of SEQ ID NO: 75. In some embodiments, the present disclosure provides a VHH sdAb that specifically binds to DLL3 and comprises a CDR1 of SEQ ID NO: 77, a CDR2 of SEQ ID NO: 78, and a CDR3 of SEQ ID NO: 79.

In some embodiments, the present disclosure provides a VHH sdAb that specifically binds to DLL3 and comprises a CDR1 of SEQ ID NO: 46, a CDR2 of SEQ ID NO: 47, and a CDR3 of SEQ ID NO: 48. In some embodiments, the present disclosure provides a VHH sdAb that specifically binds to DLL3 and comprises a CDR1 of SEQ ID NO: 52, a CDR2 of SEQ ID NO: 53, and a CDR3 of SEQ ID NO: 54.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to an amino acid sequence selected from the group consisting of SEQ ID NOs: 4, 5, 9, 13, 17, 21, 25, 29, 32, 35, 38, 42, 45, 49, 55, 59, 63, 64, 68, 70, 72, and 76. In some embodiments, the present disclosure provides a VHH sdAb that specifically binds to DLL3 and comprises or consists of an amino acid sequence selected from the group consisting of SEQ ID NOs: 4, 5, 9, 13, 17, 21, 25, 29, 32, 35, 38, 42, 45, 49, 55, 59, 63, 64, 68, 70, 72, and 76. Exemplary VHH sdAb amino acid sequence are shown in Table 2.

In some embodiments, the present disclosure provides a heavy chain variable domain that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 80. In some embodiments, the present disclosure provides a heavy chain variable domain that specifically binds to DLL3 and comprises or consists of an amino acid sequence according to SEQ ID NO: 80. In some embodiments, the present disclosure provides an antibody that specifically binds to DLL3 and comprises a heavy chain variable domain and a light chain variable domain, wherein the heavy chain variable domain comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 80.

TABLE 2
Exemplary anti-DLL3 VHH amino acid sequences

| Construct | | SEQ |
|---|---|---|
| ID | Sequence | ID |
| 2DLT10 | QVQLQESGGGLVQPGESLRLSCAASGSIVGDYAMGWYRQAPGKKRELVAGIGSEGSRDYAD | 4 |
| | SVKGRFTISRDNAKRTLYLQMNSLKSEDTAVYICFLYNSGEDYWGQGTQVTVSS | |
| 1DLT176 | QVQLQESGGGLVQPGGSLRLACAASGSIVGDYAMGWYRQAPGKKRELVAGIGSEGSRDYAD | 5 |
| | SVKGRFTISRDNAKRTLYLQMNSLKSEDTAVYICFLYNSGEDYWGQGTQVTVSS | |
| 2HCE138 | QVQLQESGGGLVQAGGSLRLSCAVSGFTTDDYGIGWFRQAPGKQRELVAIITTGGSTNYAD | 9 |
| | SVKGRFKISRDNAKNTVYLQMNSLKPEDTAVYYCNAVCSGSGCYEVSWESYDYWGQGTQVT | |
| | VSS | |
| 3HCE7 | QVQLQESGGGLVQAGDSLRLSCAGSGRTYSNYFIDWFRQAPGKEREFVAAVSWSGDRTTYA | 13 |
| | DSVKGRFTVSRDNAKNTWYLQMNSLKPEDTAVYYCAAGPLINRINYWGQGTQVTVSS | |
| 2HCE151 | QVQLQESGGGLVQPGGSLRLSCVASGFTFSSYAMSWVRQAPGKGPEWVSRIDSGGGSTSYA | 17 |
| | DSVKGRFTISRDNAKNTLHLQMNGLKPEDTAVYYCAKEPWVVADYWGQGTQVTVSS | |
| 3HCE4 | QVQLQESGGGLVQAGASLKLSCAVSGRAGSSYDMGWLRQAPGKEREFVAIITWSGNTAYKD | 21 |
| | SVKGRFIISRDNAKNTVYLEMNSLAPEDTAVYYCAAALSEKKYEYWGQGTQVTVSS | |
| 3HCE80 | QVQLQESGGGLVQPGGSLRLSCAASGSISSIISMGWYRQAPGKQRGELVAAITSGGSTSYA | 25 |
| | GSVEGRFAISRDSAKNTAYLQMNSLKPEDTAVYYCNAHVRDYSGSAYYTGQGTQVTVSS | |
| 3HCE86 | QVQLQESGGGLVQPGGSLRLSCAASGGTFSNYDIAWFRQAPGKEREFVAAVNRYGDYSYYA | 29 |
| | DSVKGRFTISRDNAKSTVSLQMNSLKPEDTFSYYCAARLWNSAKYAYWGQGTQVTVSS | |
| 3DLT81 | QVQLQESGGGLVQPGGSLRLSCAASGSIFSIYAMGWYRQAPGKKRELVAGIGSEGSRDYAD | 32 |
| | SVKGRFTISRDNAKRTVYLQMNTLQPEDTAVYYCYAGRPPSASYSGAHYWGQGTQVTVSS | |
| 3HCE38 | QVQLQESGGGLVQPGGSLRLSCAASGFTFDDVAMSWVRQAPGKQRELVAVITSGGSTYTAD | 35 |
| | SVKGRFAISRDNAKNTVYLQMNSLKPEDTALYYCAARRDSRGQYHDWGQGTQVTVSS | |
| 2HCE6 | QVQLQESGGGLVQTGGSLRLSCAASGFTFSNSPMSWVRRAPGKGPEWVSAISSNGRNTSYA | 38 |
| | DSVKGRFTISRDNAKNTLFLQMHSLKFEDTAVYYCAKGATPSVLYDYGGQGTQVTVSS | |
| 1DLT39 | QVQLQESGGGLVQPGGSLRLSCAASGRIFSMFPMGWYRQAPGKQRELVADITTGGRPNYAD | 42 |
| | SVKGRFTITRDNAKNTVYLQMNSLQPEDTAVYFCNAVSTPELAYPYDYWGQGTQVTVSS | |
| 3HCE25 | QVQLQESGGGLVQPGGSLRLSCEASGFTFSSYAMSWVRQAPGKGPEWVSRINSGGGSTSYA | 45 |
| | DSVKGRFTISRDNAKNTLYLQMNSLKPEDTAVYYCAKEPWVTQGSWGQGTQVTVSS | |
| 2HCE27 | QVQLQESGGGLVQAGGSLRLSCAASGRTFRSYAMGWFRQAPGKEREFIAVIIMSDGSTSYA | 49 |
| | DSVKGRFTISRDNAKNTLYLQMNSLKPEDTAVYYCAARRDYFTGVYDYWGQGTQVTVSS | |
| 3HCE56 | QVQLQESGGGLVQAGGSLRLSCAASGRTFSVDAMGWLRQAPGKEREFVVVIDWTGGSTAYA | 55 |
| | DSVKGRFTISRDNARNTVYLQMNNLKPEDTGVYYCAARERSRTAYDYWGQGTQVTVSS | |
| 2DLT2 | QVQLQESGGGLVQPGGSLRLSCAASGSIVGNYAMGWYRQAPGKERELVAGIGSEGSRDYAD | 59 |
| | SVKGRFTISRDTAKRTVYLQMNSLKSEDTAVYICFLYNSGDDYWGQGTQVTVSS | |
| 3HCE44 | QVQLQESGGGSVQAGGSLRLSCAASGSIFSINSMGWHRHAPGKQRELVAAKASDGSTNYAG | 63 |
| | PVRGRFTITSDDAKNTVYLQMNNLKPEDTAVYYCFLYANNIPYWAKGTQVTVSS | |
| 2HCE174 | QVQLQESGGGSVQAGGSLRLSCAASGSIFSINSMGWHRHAPGKQRELVAAKASDGSTNYAG | 64 |
| | PVRGRFTITSDDAKNTVYLQMNSLKPEDTAVYYCFLYANNIPYWAKGTQVTVSS | |
| 2HCE167 | QVQLQESGGGLVQAGGSLRLSCAASGRTFSSYAMGWLRQAPGKEREFVAVISGSGYSASYR | 68 |
| | DSVKGRFTISRDNSKNTVFLQMNSLKPEDTAVYYCAARNERGASSSYDYWGQGTQVTVSS | |
| 3HCE18 | QVQLQESGGGLVQAGGSLRLSCAASGSIFSIITMGWYRQAPGKQRGELVAAITSGGSTSYA | 70 |
| | DSVKGRFAISRDSAKNTAYLQMNSLKPEDTAVYYCNAHVRDYSGSAYYTGQGTQVTVSS | |
| 2HCE117 | QVQLQESGGGLVQPGGSLRLSCAASGFTFSSYAMSWVRQAPGKGLEWVSDINSGGGSTYYA | 72 |
| | DSVKGRFTISRDNAKNTLYLQMDSLKPEDTAVYYCATPFEIGSWGQGTQVTVSS | |
| 3HCE87 | QVQLQESGGGLVQAGGSLRLSCAASGFIFDDYIIGWFRQAPGKEREFVAAISWSGSATAYA | 76 |
| | DSVKGRFTISRDNAKNTLYLQMNTLKPEDTAVYYCAASSRGPYNSGSSYDYWGQGTQVTVS | |
| | S | |
| Anti- | QIQLVQSGPELKKPGESVKISCKASGYTFTDYAMHWVKQAPGKGLKWMGWINTYTGKPTYA | 80 |
| DLL3-1 | DDFKGRFVLSLEASASTSTLQISDLRNEDTAIYFCSRERGYYDYSRSDWGQGTLVTVSS | |

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 4. In some embodiments, the present disclosure provides a VHH sdAb that specifically binds to DLL3 and comprises or consists of SEQ ID NO: 4.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 5. In some embodiments, the present disclosure provides a VHH sdAb that specifically binds to DLL3 and comprises or consists of SEQ ID NO: 5.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 9. In some embodiments, the present disclosure provides a VHH sdAb that specifically binds to DLL3 and comprises or consists of SEQ ID NO: 9.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 13. In some embodiments, the present disclosure provides a VHH sdAb that specifically binds to DLL3 and comprises or consists of SEQ ID NO: 13.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 17. In some embodiments, the present disclosure provides a VHH sdAb that specifically binds to DLL3 and comprises or consists of SEQ ID NO: 17.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 21. In some embodiments, the present disclosure provides a VHH sdAb that specifically binds to DLL3 and comprises or consists of SEQ ID NO: 21.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 25. In some embodiments, the present disclosure provides a VHH sdAb that specifically binds to DLL3 and comprises or consists of SEQ ID NO: 25.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 29. In some embodiments, the present disclosure provides a VHH sdAb that specifically binds to DLL3 and comprises or consists of SEQ ID NO: 29.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 32. In some embodiments, the present disclosure provides a VHH sdAb that specifically binds to DLL3 and comprises or consists of SEQ ID NO: 32.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 35. In some embodiments, the present disclosure provides a VHH sdAb that specifically binds to DLL3 and comprises or consists of SEQ ID NO: 35.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 38. In some embodiments, the present disclosure provides a VHH sdAb that specifically binds to DLL3 and comprises or consists of SEQ ID NO: 38.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 42. In some embodiments, the present disclosure provides a VHH sdAb that specifically binds to DLL3 and comprises or consists of SEQ ID NO: 42.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 45. In some embodiments, the present disclosure provides a VHH sdAb that specifically binds to DLL3 and comprises or consists of SEQ ID NO: 45.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 49. In some embodiments, the present disclosure provides a VHH sdAb that specifically binds to DLL3 and comprises or consists of SEQ ID NO: 49.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 55. In some embodiments, the present disclosure provides a VHH sdAb that specifically binds to DLL3 and comprises or consists of SEQ ID NO: 55.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 59. In some embodiments, the present disclosure provides a VHH sdAb that specifically binds to DLL3 and comprises or consists of SEQ ID NO: 59.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 63. In some embodiments, the present disclosure provides a VHH sdAb that specifically binds to DLL3 and comprises or consists of SEQ ID NO: 63.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 64. In some embodiments, the present disclosure provides a VHH sdAb that specifically binds to DLL3 and comprises or consists of SEQ ID NO: 64.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 68. In some embodiments, the present disclosure provides a VHH sdAb that specifically binds to DLL3 and comprises or consists of SEQ ID NO: 68.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 70. In some embodiments, the present disclosure provides a VHH sdAb that specifically binds to DLL3 and comprises or consists of SEQ ID NO: 70.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 72. In some embodiments, the present disclosure provides a VHH sdAb that specifically binds to DLL3 and comprises or consists of SEQ ID NO: 72.

In some embodiments, the present disclosure provides a VHH sdAb that specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 76. In some embodiments, the present disclosure provides a VHH sdAb that specifically binds to DLL3 and comprises or consists of SEQ ID NO: 76.

In some embodiments, the VHH sdAb described herein are humanized. Humanized antibodies and antigen-binding fragments thereof (e.g., VHHs) have the same or similar binding specificity and affinity as a mouse or other nonhuman antibody that provides the starting material for construction of the humanized binding construct. In some embodiments, the humanized antibodies and antigen-binding fragments thereof comprise a human framework region sequence. In some embodiments, the human framework region sequence comprises or consists of an FR1 sequence according to SEQ ID NO: 745, an FR2 sequence according to any one of SEQ ID NO: 746-747, an FR3 sequence according to any one of SEQ ID NO: 748-752, and an FR4 sequence according to SEQ ID NO: 753, or a polypeptide sequence having at least 80%, at least 85%, at least 90%, at least 95%, at least 97%, at least 98%, or at least 99% identity to any one of the FR1/FR2/FR3/FR4 combinations described herein.

In some embodiments, the VHH sdAbs described herein comprise a human framework region sequence and a CDR1, a CDR2, and a CDR3, wherein the CDR1 comprises an amino acid sequence selected from SEQ ID NOs: 1, 6, 10, 14, 18, 22, 26, 30, 33, 36, 39, 46, 52, 60, 65, 69, 73, 77, and 83, the CDR2 comprises an amino acid sequence selected from SEQ ID NOs: 2, 7, 11, 15, 19, 23, 27, 40, 43, 47, 53, 61, 66, 74, 78, 81, and 82, and the CDR3 comprises an amino acid sequence selected from SEQ ID NOs: 3, 8, 12, 16, 20, 24, 28, 31, 34, 37, 41, 44, 48, 54, 62, 67, 71, 75, 79, and 84. Exemplary humanized VHH sdAb amino acid sequences are provided in Table 3 below.

TABLE 3
Humanized Anti-DLL3 VHH Sequences

| Construct | | SEQ |
|---|---|---|
| ID | Sequence | ID |
| 2HCE27 | EVQLVESGGGLVQPGGSLRLSCAASGRTFRSYAMGWFRQAPGKEREFIAVIIMSDGSTSYA | 50 |
| 14.1 | DSVKGRFTISRDNAKNTLYLQMNSLKPEDTAVYYCAARRDYFTGVYDYWGQGTLVTVSS | |
| 2HCE27 | EVQLVESGGGLVQPGGSLRLSCAASGRTFRSYAMGWFRQAPGKEREFIAVIIMSDGSTSYA | 51 |
| 14.2 | DSVKGRFTISRDNAKNTLYLQMNSLRPEDTAVYYCAARRDYFTGVYDYWGQGTLVTVSS | |
| 3HCE56 | EVQLVESGGGLVQPGGSLRLSCAASGRTFSVDAMGWLRQAPGKEREFVVVIDWTGGSTAYA | 56 |
| 15.1 | DSVKGRFTISRDNARNTVYLQMNNLKPEDTGVYYCAARERSRTAYDYWGQGTLVTVSS | |
| 3HCE56 | EVQLVESGGGLVQPGGSLRLSCAASGRTFSVDAMGWLRQAPGKEREFVVVIDWTGGSTAYA | 57 |
| 15.2 | DSVKGRFTISRDNARNTVYLQMNSLRPEDTAVYYCAARERSRTAYDYWGQGTLVTVSS | |
| 3HCE56 | EVQLVESGGGLVQPGGSLRLSCAASGRTFSVDAMGWLRQAPGKEREFVVVIDWTGGSTAYA | 58 |
| 15.3 | DSVKGRFTISRDNAKNTVYLQMNSLRPEDTAVYYCAARERSRTAYDYWGQGTLVTVSS | |

In some embodiments, the humanized VHH sdAb specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 50. In some embodiments, the present disclosure provides a VHH sdAb that specifically binds to DLL3 and comprises or consists of SEQ ID NO: 50.

In some embodiments, the humanized VHH sdAb specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 51. In some embodiments, the present disclosure provides a VHH sdAb that specifically binds to DLL3 and comprises or consists of SEQ ID NO: 51.

In some embodiments, the humanized VHH sdAb specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 56. In some embodiments, the present disclosure provides a VHH sdAb that specifically binds to DLL3 and comprises or consists of SEQ ID NO: 56.

In some embodiments, the humanized VHH sdAb specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 57. In some embodiments, the present disclosure provides a VHH sdAb that specifically binds to DLL3 and comprises or consists of SEQ ID NO: 57.

In some embodiments, the humanized VHH sdAb specifically binds to DLL3 and comprises an amino acid sequence that is at least 85%, at least 90%, at least 91%, at least 92%, at least 93%, at least 94%, at least 95%, at least 96%, at least 97%, at least 98%, at least 99%, at least 99.5%, or 100% identical to SEQ ID NO: 58. In some embodiments, the present disclosure provides a VHH sdAb that specifically binds to DLL3 and comprises or consists of SEQ ID NO: 58.

In some embodiments, the present disclosure provides bispecific binding constructs comprising at least a first binding domain and a second binding domain, wherein the first binding domain binds to a first antigen or target (e.g., DLL3), and the second binding domain binds to a second antigen or target. In some embodiments, a bispecific binding construct of the disclosure may be a bispecific antibody of the disclosure.

Bispecific binding constructs of the disclosure may include, but are not limited to, formats such as DVD-Ig, mAb2, FIT-Ig, mAb-dAb, dock and lock, Fab-arm exchange, SEEDbody, Triomab, LUZ-Y, Fcab, KX-body, orthogonal Fab, scDiabody-Fc, diabody-Fc, tandem scFv-Fc, Fab-scFv-Fc, Fab-scFv, intrabody, BITE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody. In some embodiments, at least one Fab or scFv in any one of the bispecific formats as described in this paragraph is replaced by a VHH (e.g., an anti-DLL3 VHH of the present disclosure).

In some embodiments, the anti-DLL3 sdAbs described herein are linked or directly fused to a second domain to form a bispecific binding construct. In some embodiments, such binding constructs comprise, from N- to C-terminus, anti-DLL3 sdAb—second domain, second domain—anti-DLL3 sdAb, anti-DLL3 sdAb—linker—second domain, or second domain—linker anti-DLL3 sdAb.

As used herein, the term “linker” generally refers to a short polypeptide sequence connecting two sub-domains of a polypeptide. Non-limiting examples of linkers include flexible linkers comprising glycine-serine repeats, and linkers derived from (a) an interdomain region of a transmembrane protein (e.g., a type I transmembrane protein); (b) a stalk region of a type II C-lectin; or (c) an immunoglobulin hinge. In some embodiments, a linker provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids. Exemplary linkers are shown in Table 4.

TABLE 4
Exemplary linker Sequences

| | | SEQ |
|---|---|---|
| Linker | Amino Acid Sequence | ID |
| STD1 | NYGGGGSGGGGSGGGGSGNS | 85 |
| STD2 | NYGGGGSGGGGSGGGGSGNYGGGGSGGGGSGGGGSGNS | 86 |
| H1 | NS | 87 |
| H2 | GGGGSGNS | 88 |
| H3 | NYGGGGSGNS | 89 |
| H4 | GGGGSGGGGSGNS | 90 |
| H5 | NYGGGGSGGGGSGNS | 91 |
| H6 | GGGGSGGGGSGGGGSGNS | 92 |
| H7 | GCPPCPNS | 93 |
| Gly₄Ser | GGGGS | 94 |
| (G₄S)₃ | GGGGSGGGGSGGGGS | 95 |
| H105 | SGGGGSGGGGSGGGGS | 96 |
| (G₄S)₄ | GGGGSGGGGSGGGGSGGGGS | 97 |
| H75 (NKG2A | QRHNNSSLNTGTQMAGHSPNS | 98 |
| quadruple mutant) | | |
| H83 (NKG2A | SSLNTGTQMAGHSPNS | 99 |
| derived) | | |
| H106 (NKG2A | QRHNNSSLNTGTQMAGHS | 100 |
| derived) | | |
| H81 (NKG2D | EVQIPLTESYSPNS | 101 |
| derived) | | |
| H91 (NKG2D | NSLANQEVQIPLTESYSPNS | 102 |
| derived) | | |
| H94 | SGGGGSGGGGSGGGGSPNS | 103 |
| H111 | SGGGGSGGGGSGGGGSPGS | 104 |
| H113 | SGGGGSGGGGSGGGGSPAS | 105 |
| H114 | SGGGGSGGGGSGGGGSPS | 106 |
| H115 | SGGGGSGGGGSGGGGSPSS | 107 |
| H116 | SGSETPGTSESATPES | 108 |
| H117 | GGGGSGGGGSGGGGS | 109 |
| H118 | AHHSEDPSSKAPKAP | 110 |
| H119 | SPSTPPTPSPSTPPAS | 111 |

In some embodiments, the VHH sdAbs described herein are linked or directly fused to a second binding domain. Therefore, in some embodiments, the present disclosure provides bispecific binding constructs comprising an anti-DLL3 sdAb described herein and a second binding domain. In some embodiments, the second binding domain is an antigen binding domain. In some embodiments, the antigen binding domain is selected from an antigen binding domain that specifically binds to CD3, an antigen binding domain that specifically binds to human serum albumin (HSA). In some embodiments, the second binding domain is an Fc domain. In some embodiments, the bispecific binding constructs specifically bind to DLL3 and CD3. In some embodiments, the bispecific binding constructs comprise a VHH that specifically binds to DLL3 and an scFv that specifically binds to CD3. In some embodiments, the bispecific binding constructs comprise a VHH that specifically binds to DLL3 and a VH-VL pairing that specifically binds to CD3. In some embodiments, the bispecific binding constructs comprise an Fc domain. In some embodiments, the bispecific binding constructs comprise an Fc domain having a knobs-in-holes (KIH) format. In some embodiments, the Fc KIH format comprise an amino acid substitution when compared to a wild type Fc amino acid sequence. In some embodiments, the CD3 antigen binding domain comprises one or more CDRs selected from those in Table 6.

In some embodiments, the bispecific binding constructs described herein comprise an anti-DLL3 sdAb linked or directly fused to a second antigen binding domain. In some embodiments, the second antigen binding domain specifically binds to human serum albumin (HSA) or CD3. In some embodiments, the anti-DLL3 single domain antibody is a VHH antibody domain and the second antigen-binding domain is an scFv. Such embodiments are also referred to as light T-cell engagers (LiTEs) (See e.g., Molgaard et al., Cancer Immunol Immunother. 2018 August; 67(8):1251-1260). In some embodiments, the HSA antigen binding domain comprises one or more CDRs selected from those in Table 5. In some embodiments, the CD3 antigen binding domain comprises one or more CDRs selected from those in Table 6

In some embodiments, the bispecific binding constructs described herein comprise an anti-DLL3 sdAb linked or directly fused to a second antigen-specific binding domain that specifically binds to a cell surface protein found on natural killer (NK) cells (e.g., CD16 or NKp46). In some embodiments, the anti-DLL3 single domain antibody is a VHH antibody domain and the second antigen-binding domain is an scFv. NKp46, also known as CD335, belongs to the natural cytotoxicity receptor (NCR) family and is a glycoprotein with 2 Ig-like domains and a short cytoplasmic tail. CD16, also known as FcγRIII, is a cluster of differentiation molecule found on the surface of natural killer cells, neutrophils, monocytes, and macrophages. In some embodiments, the CD16 antigen binding domain comprises one or more CDRs selected from those in Table 7.

In some embodiments, the present disclosure provides trispecific binding constructs that specifically bind DLL3. Trispecific binding constructs comprise at least a first binding domain, a second binding domain, and a third binding domain, wherein at least one of the binding domains is an antigen binding domain that specifically binds to DLL3. In some embodiments, the first, second, and third binding domains are antigen binding domains, wherein the first antigen binding domain specifically binds to DLL3 the second binding domain binds to a second antigen or target, and the third binding domain binds to a third antigen or target. Each antigen binding domain may be a VHH, an scFv, an Fab, or other antigen binding domain format, and the three binding domains may be of different format (e.g., VHH or scFv or Fab). In some embodiments, the trispecific binding construct comprises two arms with each arm comprising two different VHH/scFv/Fab linked in tandem and each VHH/scFv/Fab specifically binds to a distinct antigen, an IgG constant region that links to the arms at one end, and a third VHH/scFv/Fab specifically binding to a third antigen linked to the other end of the IgG constant domain. In some embodiments, the trispecific binding construct comprises two arms and an IgG constant region that links to the arms, one of the arm comprises two different VHH/scFv/Fab linked in tandem and each VHH/scFv/Fab specifically binds to a distinct antigen, and the other arm comprises a third VHH/scFv/Fab that specifically binds to a third antigen. In some embodiments, three different VHH/scFv/Fab are linked in tandem, and each VHH/scFv/Fab specifically binds a distinct antigen. In some embodiments, one of the VHH/scFv/Fab of the trispecific binding construct is replaced by an Fc domain. Exemplary formats of trispecific antibodies include those disclosed in Runcie et al., Mol Med. 2018 September s24; 24(1):50; Wu et al., Nature Cancer 2020, volume 1, p 86-98; Wu and Demarest, Methods. 2019 Feb. 1; 154:3-9. In some embodiments, at least one binding domain of the trispecific binding construct is an anti-DLL3 VHH of the present disclosure.

In some embodiments, the trispecific binding constructs comprise a first antigen binding domain comprising an anti-DLL3 sdAb, a second binding domain, and a third binding domain. In some embodiments, the second binding domain and/or the third binding domain of the trispecific binding construct are antigen binding domains comprising a VHH or an scFv. In some embodiments, the second binding domain of the trispecific binding construct is an antigen binding domain that specifically binds to human serum albumin (HSA). In some embodiments, the third binding domain is an antigen binding domain that specifically binds to CD3, CD16, or NKp46. In some embodiments, the second binding domain of the trispecific binding construct comprises an Fc domain that binds to Fc receptors. In some embodiments, the trispecific binding constructs comprise three antigen binding domains specifically binding, individually, to DLL3, HSA and CD3. In some embodiments, the trispecific binding construct comprises a VHH that specifically binds to DLL3, a VHH that specifically binds to HSA and an scFv that specifically binds to CD3. In some embodiments, the trispecific binding constructs comprise a first antigen binding domain that specifically binds to DLL3, a second antigen binding domain that specifically binds to CD3, and a third binding domain comprising an Fc domain. In some embodiments, the trispecific binding construct comprises a VHH that specifically binds to DLL3, an Fc domain, and an scFv or a VH-VL pairing that specifically binds to CD3. In some embodiments, the Fc domain comprises a knobs-in-holes (KIH) format or at least one amino acid substitution compared to a wild type Fc that confers a KIH format.

In some embodiments, the trispecific binding construct comprises a VHH that specifically binds to DLL3, a second binding domain that specifically binds to HSA, and a third binding domain that specifically binds to CD3.

Exemplary HSA antigen binding domains suitable for use according to the present disclosure include, but are not limited to, those comprising CDR1, CDR2 and CDR3 sequences as shown in Table 5 or described in U.S. Pat. No. 8,188,223. In some embodiments, the HSA binding domain of the disclosure comprises a CDR1, a CDR2, and a CDR3 according to Table 5.

TABLE 5
Exemplary anti-HSA CDR sequences

| | CDR1 | SEQ ID: | CDR2 | SEQ ID: | CDR3 | SEQ ID: |
|---|---|---|---|---|---|---|
| HSA-1 | SFGMS | 113 | SISGSGSDTLYADSVKG | 114 | GGSLSR | 115 |
| HSA-2 | LNLMG | 116 | TITVGDSTNYADSVKG | 117 | RRTWHSEL | 118 |
| HSA-3 | INLLG | 119 | TITVGDSTSYADSVKG | 120 | RRTWHSEL | 121 |
| HSA-4 | SFGMS | 122 | SINGRGDDTRYADSVKG | 123 | GRSVSRS | 124 |
| HSA-5 | SFGMS | 125 | AISADSSDKRYADSVKG | 126 | GRGSP | 127 |
| HSA-6 | SFGMS | 128 | AISADSSDKRYADSVKG | 129 | GRGSP | 130 |
| HSA-7 | NYWMY | 131 | RISTGGGYSYYADSVKG | 132 | DREAQVDTLDFDY | 133 |

Exemplary CD3 antigen binding domain suitable for use according to the present disclosure include, but are not limited to, those described in WO 2008/119567, U. S. Pat. Pub. Nos: US2016/0176973, US2013/0156770, US2020/0071405, US2019/0040135, US2018/0355038, U.S. Pat. No. 10,174,124. Exemplary heavy chain variable domain (CD3-VH) and light chain variable domain (CD3-VL) CDRs that specifically bind to CD3 are shown in Table 6. In some embodiments, the CD3 binding domain of the disclosure comprises a heavy chain variable domain (CD3-VH) comprising a CDR1, a CDR2, and a CDR3 according to Table 6, and/or a light chain variable domain (CD3-VL) comprising a CDR1, a CDR2, and a CDR3 according to Table 6.

TABLE 6
Exemplary anti-CD3 CDR sequences

| | | SEQ | | SEQ | | SEQ |
|---|---|---|---|---|---|---|
| | CDR1 | ID: | CDR2 | ID: | CDR3 | ID: |
| CD3- | GSSTGAVTSGY | 474 | GTKFLAP | 475 | ALWYSNRWV | 476 |
| VL-1 | YPN | | | | | |
| CD3- | RSSTGAVTSGYY | 477 | ATDMRPS | 478 | ALWYSNRWV | 479 |
| VL-2 | PN | | | | | |
| CD3- | GSSTGAVTSGN | 480 | GTKFLAP | 481 | VLWYSNRWV | 482 |
| VL-3 | YPN | | | | | |
| CD3- | IYAMN | 483 | RIRSKYNNYATYYADS | 484 | HGNFGNSYVSFFAY | 485 |
| VH-1 | | | VKS | | | |
| CD3- | KYAMN | 486 | RIRSKYNNYATYYADS | 487 | HGNFGNSYISYWAY | 488 |
| VH-2 | | | VKD | | | |
| CD3- | SYAMN | 489 | RIRSKYNNYATYYADS | 490 | HGNFGNSYLSFWAY | 491 |
| VH-3 | | | VKG | | | |
| CD3- | RYAMN | 492 | RIRSKYNNYATYYADS | 493 | HGNFGNSYLSYFAY | 494 |
| VH-4 | | | VKG | | | |
| CD3- | VYAMN | 495 | RIRSKYNNYATYYADS | 496 | HGNFGNSYLSWWA | 497 |
| VH-5 | | | VKK | | Y | |
| CD3- | KYAMN | 498 | RIRSKYNNYATYYADS | 499 | HGNFGNSYTSYYAY | 500 |
| VH-6 | | | VKS | | | |
| CD3- | GYAMN | 501 | RIRSKYNNYATYYADS | 502 | HRNFGNSYLSWFAY | 503 |
| VH-7 | | | VKE | | | |
| CD3- | VYAMN | 504 | RIRSKYNNYATYYADS | 505 | HGNFGNSYISWWAY | 506 |
| VH-8 | | | VKK | | | |
| CD3- | SYAMN | 507 | RIRSKYNNYATYYADS | 508 | HGNFGNSYVSWWA | 509 |
| VH-9 | | | VKG | | Y | |
| CD3- | KYAMN | 510 | RIRSKYNNYATYYADS | 511 | HGNFGNSYISYWAY | 512 |
| VH-10 | | | VKD | | | |
| CD3- | SSTGAVTTSNYA | 513 | GTNKRA | 514 | LWYSNLWV | 515 |
| VL-4 | N | | | | | |
| CD3- | TYAMN | 516 | IRSKYNNYATYYADSV | 517 | HGNFGNSYVSWFAY | 518 |
| VH-11 | | | KD | | | |
| CD3- | GFTFDDYS | 519 | ISWNSGSK | 520 | AKYGSGYGKFYHY | 521 |
| VH-12 | | | | | GLDV | |
| CD3- | GFTFDDYA | 522 | ISWNSGSI | 523 | AKDGSGYGYFYYY | 524 |
| VH-13 | | | | | GMDV | |
| CD3- | GFTFDDYS | 525 | ISWNSGSI | 526 | AKDGSGYGYFYYY | 527 |
| VH-14 | | | | | GMDV | |
| CD3- | GFTFDDYA | 528 | ISWNSGSI | 529 | AKDGSGYGKFYYY | 530 |
| VH-15 | | | | | GMDV | |
| CD3- | GFTFDDYS | 531 | ISWNSGSI | 532 | AKDGSGYGKFYYY | 533 |
| VH-16 | | | | | GMDV | |
| CD3- | GFTFDDYS | 534 | ISWNSGSI | 535 | AKYGSGYGKFYHY | 536 |
| VH-17 | | | | | GLDV | |
| CD3- | GFTFDDYS | 537 | ISWNSGSK | 538 | AKDGSGYGKFYHY | 539 |
| VH-18 | | | | | GLDV | |
| CD3- | GFTFDDYS | 540 | ISWNSGSK | 541 | AKYGSGYGKFYYY | 542 |
| VH-19 | | | | | GLDV | |
| CD3- | GFTFDDYS | 543 | ISWNSGSK | 544 | AKYGSGYGKFYHY | 545 |
| VH-20 | | | | | GMDV | |
| CD3- | GFTFDDYS | 546 | ISWNSGSI | 547 | AKDGSGYGKFYHY | 548 |
| VH-21 | | | | | GLDV | |
| CD3- | GFTFDDYS | 549 | ISWNSGSI | 550 | AKYGSGYGKFYYY | 551 |
| VH-22 | | | | | GLDV | |
| CD3- | GFTFDDYS | 552 | ISWNSGSI | 553 | AKYGSGYGKFYHY | 554 |
| VH-23 | | | | | GMDV | |
| CD3- | GFTFDDYS | 555 | ISWNSGSK | 556 | AKDGSGYGKFYYY | 557 |
| VH-24 | | | | | GLDV | |
| CD3- | GFTFDDYS | 558 | ISWNSGSK | 559 | AKDGSGYGKFYHY | 560 |
| VH-25 | | | | | GMDV | |
| CD3- | GFTFDDYS | 561 | ISWNSGSK | 562 | AKYGSGYGKFYYY | 563 |
| VH-26 | | | | | GMDV | |
| CD3- | GFTFDDYS | 564 | ISWNSGSI | 565 | AKDGSGYGKFYYY | 566 |
| VH-27 | | | | | GLDV | |
| CD3- | GFTFDDYS | 567 | ISWNSGSI | 568 | AKDGSGYGKFYHY | 569 |
| VH-28 | | | | | GMDV | |
| CD3- | GFTFDDYS | 570 | ISWNSGSI | 571 | AKYGSGYGKFYYY | 572 |
| VH-29 | | | | | GMDV | |
| CD3- | GFTFDDYS | 573 | ISWNSGSK | 574 | AKDGSGYGKFYYY | 575 |
| VH-30 | | | | | GMDV | |
| CD3- | GFTFDDYS | 576 | ISWNSGSK | 577 | AKYGSGYGKFYHY | 578 |
| VH-31 | | | | | GLDV | |
| CD3- | NYYIH | 579 | WIYPGDGNTKYNEKFK | 580 | DSYSNYYFDY | 581 |
| VH-32 | | | G | | | |
| CD3- | NYYIH | 579 | WIYPGDGNTKYNEKFK | 580 | DSYSNYYFDY | 581 |
| VH-n | | | G | | | |
| | SYYIH | 582 | WIYPENDNTKYNEKFK | 583 | DGYSRYYFDY | 584 |
| | | | D | | | |
| | GYTMN | 585 | LINPYKGVSTYNQKFK | 586 | DAYSRYYFDY | 587 |
| | | | D | | | |
| | TYAMN | 588 | LINPYKGVTTYADSVK | 589 | TGYSRYYFDY | 590 |
| | | | G | | | |
| | NYGMN | 591 | LINPYKGVSTYADSVK | 592 | DGYSRYAFDY | 593 |
| | | | G | | | |
| | SYYIH | 594 | RIRSKYNNYATYYADS | 595 | SGYSRYYFDY | 596 |
| | | | VKD | | | |
| | NYAIH | 597 | WINTNTGKPTYAEEFK | 598 | DGYSRAYFDY | 599 |
| | | | G | | | |
| | GFSLTNYAIH | 600 | WIYPGNVNTKYNEKFK | 601 | DSYSNYYFDY | 602 |
| | | | G | | | |
| | GSAMH | 603 | VIWAGGNTKYNSALMS | 604 | SGYYGDSDWYFDV | 605 |
| | SDYIH | 606 | GVIWAGGNTK | 607 | HGNFGNSYVSWFAY | 608 |
| | | | YNSALMS | | | |
| | SHYLH | 609 | RIRSRANSYATAYAAS | 610 | RGDYRYAWFLY | 611 |
| | | | VKD | | | |
| | SYYIH | 612 | WIYFGNVNTKYNEKFK | 613 | NHDYYFDY | 614 |
| | | | G | | | |
| | SYYIH | 615 | WINPGDGNVKYNEKFK | 616 | EDSSGYVALDY | 617 |
| | | | D | | | |
| | NYGMN | 618 | WIFPGSDNTKYNEKFK | 619 | QMNSLRAEDTAVY | 620 |
| | | | G | | YCAREDSSGYVALD | |
| | | | | | Y | |
| | NYYIH | 621 | WIYPGNVNTKYNEKFK | 622 | DTMVRGIDY | 623 |
| | | | G | | | |
| | SYWMH | 624 | WINTNTGKPTYAEDFK | 625 | DGGYYFDY | 626 |
| | | | G | | | |
| | NYYMH | 627 | WIYPGSDNTKFNDKFK | 628 | DGAYYFDY | 629 |
| | | | G | | | |
| | NYYTH | 630 | NFYPGDLTVNYDEKFK | 631 | NGNYAMDY | 632 |
| | | | N | | | |
| | NYYTH | 633 | WISPGSGSIKYNEKFKG | 634 | NHDYYFDY | 635 |
| | NYYTH | 636 | WIYPGNGNIKYNEKFM | 637 | RGDYRYAWFTY | 638 |
| | | | G | | | |
| | SYWMH | 639 | WIYPGNGNIKYNEKFM | 640 | DSITNYYFDY | 641 |
| | | | G | | | |
| | SYYIH | 642 | WLYPGNGDTRYNEKFK | 643 | DAYSRYFFDY | 644 |
| | | | D | | | |
| | SYWMH | 645 | NSYPGDLNVNYDEKFK | 646 | DGYSLYFFDF | 647 |
| | | | N | | | |
| | SCAIS | 648 | WLYPGDVSTRYNEKFR | 649 | DSYGSYYFDY | 650 |
| | | | D | | | |
| | DYYIH | 651 | NIYPGGEIINYAEKFKT | 652 | DSYGSYYFDY | 653 |
| | | | FMSVTGSAYYANWAK | 654 | DSYGNYFFDY | 655 |
| | | | S | | | |
| | | | LINPYKGVXTYXXXXK | 656 | DAYSRYFFDY | 657 |
| | | | X ** | | | |
| | | | WIYPGDVSTRFNEKFK | 658 | DSSASYYFDF | 659 |
| | | | G | | | |
| | | | | | DTTGNYFFDY | 660 |
| | | | | | VGIGSGLNI | 661 |
| | | | | | XXYSXXXFDY | 662 |
| | | | | | DSYGNYFFDY | 663 |
| CD3- | KSSQSLLNSRTR | 664 | WASTRES | 665 | TQSFILRT | 666 |
| VL-5 | KNYLA | | | | | |
| CD3- | KSSQSLLNSRTR | 664 | WASTRES | 665 | TQSFILRT | 666 |
| VL-n | KNYLA | | | | | |
| | KSSQSLLNSRTR | 667 | WTSTRKS | 668 | KQSFILRT | 669 |
| | KNYLA | | | | | |
| | RASQDIRNYLN | 670 | YTSRLES | 671 | KQSFALRT | 672 |
| | RSSTGAVTTSNY | 673 | GTNKRAP | 674 | KASFILRT | 675 |
| | AN | | | | | |
| | RSSTGAVTTSNY | 676 | GTSNRAP | 677 | KQSAILRT | 678 |
| | AN | | | | | |
| | KSSQSLLNSRTR | 679 | WASTRES | 680 | TQSFILRT | 681 |
| | KNYLA | | | | | |
| | KSSQSLLSGRTR | 682 | WASTRES | 683 | QQGNTLPWT | 684 |
| | KNYLA | | | | | |
| | KSSQSLLSGRTR | 685 | WASTRES | 686 | ALWYSNLWV | 687 |
| | KNYLA | | | | | |
| | KSSQSVLYSSNN | 688 | RASTRES | 689 | ALWYSTHFV | 690 |
| | KNYLV | | | | | |
| | KSSQSLLNSRTR | 691 | WASTRES | 692 | KQSYILRT | 693 |
| | KNYLA | | | | | |
| | KSSQSLLNSRTR | 694 | WASTRES | 695 | KQSYYLLT | 696 |
| | KNYLA | | | | | |
| | KSSQSLLNSRTR | 697 | WASTRES | 698 | KQSYYLLT | 699 |
| | KNYLA | | | | | |
| | KSSQSLLNSRTR | 700 | WASTRES | 701 | QQYYSVPWT | 702 |
| | KNYLA | | | | | |
| | RSSTGAVTTSNY | 703 | GTSNRAP | 704 | KQSFTLRT | 705 |
| | AN | | | | | |
| | KSSQSLLNIRTR | 706 | WASTRYS | 707 | KQSYTLRT | 708 |
| | KNCLA | | | | | |
| | KSSQSLLNSRTR | 709 | WASTRES | 710 | KQSYTLRT | 711 |
| | KNYLA | | | | | |
| | KSSQSLLNSRTR | 712 | WASTRES | 713 | KQSYILRT | 714 |
| | KNYLA | | | | | |
| | KSSQSLLNSRTR | 715 | WASTRES | 716 | ALWYSTHFV | 717 |
| | KNYLA | | | | | |
| | KSSQSLLNSRTR | 718 | WASTRES | 719 | TQSYTLRT | 720 |
| | KNYLA | | | | | |
| | KSSQSLLNSRTR | 721 | WASTRES | 722 | CQSFILRT | 723 |
| | KNYLA | | | | | |
| | KSSQSLLNSRTR | 724 | WASTRES | 725 | KQSFILRT | 726 |
| | KNYLA | | | | | |
| | KSSQSLLNIRTR | 727 | WASTRAS | 728 | TQSFILRT | 729 |
| | KNYLA | | | | | |
| | KSSQSLLNSRTR | 730 | WASTRES | 731 | KQSFILRT | 732 |
| | KNYLA | | | | | |
| | QASETVYSNNY | 733 | GVSTLDS | 734 | KQSFILRT | 735 |
| | LA | | | | | |
| | KSSQSLLNSRTR | 736 | WASTRES | 737 | CQSFILRT | 738 |
| | KNYLA | | | | | |
| | | | | | TQSFILRT | 739 |
| | | | | | TQSFILRT | 740 |
| | | | | | AGYKTSSSYAIA | 741 |
| | | | | | XXSXXLRT | 742 |
| | | | | | CTQSFILRT | 743 |

** “X” indicates any amino acid can occur in that position.

Exemplary CD 16 antigen binding domains suitable for use according to the present disclosure include, but are not limited to, those described in U.S. Pat. No. 9,701,750, U.S. Pat. Pub. No. 2020/0010547, 2008/0145362. Exemplary heavy chain variable domain (CD16-VH) and light chain variable domain (CD 16-VL) CDRs that specifically bind to CD 16 are shown in Table 7. In some embodiments, the CD 16 antigen binding domain of the disclosure comprises a heavy chain variable domain (CD16-VH) comprising a CDR1, a CDR2, and a CDR3 according to Table 7, and/or a light chain variable domain (CD 16-VL) comprising a CDR1, a CDR2, and a CDR3 according to Table 7.

TABLE 7
Exemplary anti-CD16 CDR sequences

| | | SEQ | | SEQ | | SEQ |
|---|---|---|---|---|---|---|
| | CDR1 | ID: | CDR2 | ID: | CDR3 | ID: |
| CD16-VH | TSYYMH | 450 | IINPSGGSTSY | 451 | GSAYYYDFADY | 452 |
| | | | AQKFQG | | | |
| CD16-VL-1 | SGDKLEEKYVS | 453 | QDNKRPS | 454 | QVWDNYSVL | 455 |
| CD16-VL-2 | GGNNIESRNVH | 456 | RDNNRPS | 457 | QVWDNYTVL | 458 |
| CD16-VL-3 | GGNNIGSKNVH | 459 | RDSNRPS | 460 | QVWDNYIVL | 461 |
| CD16-VL-4 | EGNNIGSKNVH | 462 | DDSDRPS | 463 | QVWDNYSVL | 464 |
| CD16-VL-5 | GGNNIGSKNVH | 465 | RDSSRPS | 466 | QVWDDYIVV | 467 |
| CD16-VL-6 | GANDIGKRNVH | 468 | QDNKRPS | 469 | QVWDNYSVL | 470 |
| CD16-VL-7 | GGHNIGSKNVH | 471 | QDNKRPS | 472 | QVWDNYSVL | 473 |

Exemplary NKp46 antigen binding domains suitable for use according to the present disclosure include, but are not limited to, Bab281, mIgG1, available commercially from Beckman Coulter, Inc. (Brea, Calif, USA) (see Pessino et al., J Exp Med, 1998, 188(5):953-960 and Sivori et al., Eur J Immunol, 1999, 29:1656-1666, describing chromium release cytotoxicity assays). Another NKp46 binding antibody is 9E2, mIgG1, available commercially from Becton Dickinson (Franklin Lakes, N.J., USA) and Miltenyi Biotec (Bergisch Gladback, Germany) (see Brando et al. (2005) J Leukoc Biol 78:359-371 and El-Sherbiny et al. (2007) Cancer Research 67(18):8444-9). Another anti-NKp46 binding antibody is 195314, mIgG2b, available commercially from R&D Systems, Inc. (Minneapolis, USA) (see Nolte-'t Hoen et al. (2007) Blood 109:670-673). The NKp46 antigen binding domain may comprise variable region or CDR sequences from such Bab281, 9E2 or 195314 antibodies. In some embodiments, the NKp46 antigen binding domain comprises a sequence having at least 85%, at least 90%, at least 95%, at least 98%, at least 99%, or 100% identity to STGSEVQLQQSGPELVKPGASVKISCKTSGYTFTEYTMHWVKQSHGKSLEWIGGISPNIGGTSY NQKFKGKATLTVDKSSSTAYMELRSLTSEDSAVYYCARRGGSFDYWGQGTTLTVSSVEGGSGGS GGSGGSGGVDDIVMTQSPATLSVTPGDRVSLSCRASQSISDYLHWYQQKSHESPRLLIKYASQS ISGIPSRFSGSGSGSDFTLSINSVEPEDVGVYYCQNGHSFPLTFGAGTKLELK (SEQ ID NO: 744). In some embodiments, the NKp46 antigen binding domain comprises CDR1, CDR2 and CDR3 sequences that are identical to those in SEQ ID NO: 744. See, U.S. Pat. No. 10,344,087 and U.S. Pat Appl. No. 2017/0210802.

In some embodiments, the bispecific or trispecific binding constructs of the disclosure comprise an anti-DLL3 sdAb linked or directly fused to an antibody Fc domain. In some embodiments, the Fc domain is an IgG isotype (e.g. IgG1, IgG2, IgG3, IgG4). In some embodiments, the Fc domain comprises a wild-type IgG amino acid sequence. Such sequences are known in the art, see e.g. Shields et al., J Biol Chem, (2001) 276:9; 6591-6604. In some embodiments, the CH2 or CH3 domain of the Fc domain comprises one or more amino acid mutations that alter the function and/or stability of the antibody. For example, in some embodiments, the Fc domain of an anti-DLL3 antibody described herein lacks or has minimal effector functions while retaining the ability to bind some Fc receptors such as the neonatal Fc receptor (FcRn) and retaining a relatively long half-life in vivo. In some embodiments, the Fc domain is engineered to increase binding to Fc7 receptors. In some embodiments, the Fc domain has enhanced engagement of effector immune cells (e.g., Natural Killer cells, macrophages) and/or increased Fc-mediated effector functions (e.g., ADCC). In some embodiments, the Fc domain is afucosylated. In some embodiments, afucosylation of Fc domain can be achieved by engineering of the expression cell line (e.g., CHO DG44) with strategies including, but not limited to, inactivation of the FUT8 gene, loss-of-function mutations in the Golgi GDP-fucose transporter (GFT) gene (Slc35c1), and overexpression of 4-b-N-acetylglucosaminyltransferase (GnT-III). In some embodiments, afucosylation of Fc domain can be achieved by amino acid mutation in the Fc domain. For IgG1, such mutations include, but are not limited to, one or more mutations selected from T256A, K290A, S298A, E333A, and K334A. In some embodiments, the Fc variant comprises a single S239D or 1332E mutation, a double S239D/I332E mutations, or a triple S239D/I332E/A330L mutations. In some embodiments, the engineered Fc domain is an Fc variant with S239D/I332E mutations. See for example Pereira et al., MAbs. 2018 July; 10(5): 693-711; Horton et al., Cancer Res. 2008 Oct. 1; 68(19):8049-57. In some embodiments, such binding constructs with afucosylated Fc domain display enhanced ADCC activity. In some embodiments, the Fc variant of the bispecific or trispecific binding construct does not result in, or substantially reduces the induction of, antibody-dependent cell-mediated cytotoxicity (ADCC), antibody-dependent cell-mediated phagocytosis (ADCP), complement activation, and/or complement-dependent cytotoxicity (CDC). Such mutations are well known in the art, see for example Shields et al., J Biol Chem, (2001) 276:9; 6591-6604; Arduin et al., Mol Immunol (2015) 63:2; 456-463; Vafa et al., Methods (2014) 65:1; 114-126. Unless otherwise noted, the numbering of the residues in an IgG heavy chain is that of the EU index as in Kabat et al., Sequences of Proteins of Immunological Interest, 5th Ed. Public Health Service, National Institutes of Health, Bethesda, MD (1991), expressly incorporated herein by references. The “EU index as in Kabat” refers to the numbering of the human IgG1 EU antibody.

In some embodiments, the present disclosure provides an anti-DLL3 antibody comprising a heavy chain variable region (VH) and light chain variable region (VL), wherein the VH comprises a comprises a heavy chain complementarity determining region (CDR) 1 (HCDR1), an HCDR2, and an HCDR3 selected from those listed in Table 1, and wherein the VL comprises a light chain complementarity determining region (CDR) 1 (LCDR1), an LCDR2, and an LCDR3 selected from those known in the art. Light chain CDRs that specifically bind to DLL3 are known in the art, for example, see WO 2019/222283, WO 2019/222282, WO 2019/222278, U.S. Pat. Pub. Nos. 2019/0046656, 2019/0263907, 2019/0270817, and U.S. Pat. No. 10,308,721. Exemplary light chain variable domain (DLL3-VL) LCDRs that specifically bind to DLL3 are shown in Table 8. In some embodiments, the DLL3 binding domain of the disclosure comprises a light chain variable domain (DLL3-VL) comprising an LCDR1, an LCDR2, and an LCDR3 according to Table 8.

TABLE 8
Exemplary anti-DLL3 light chain CDR sequences

| | | SEQ | | SEQ | | SEQ |
|---|---|---|---|---|---|---|
| | | ID | | ID | | ID |
| | LCDR1 | NO: | LCDR2 | NO: | LCDR3 | NO: |
| DLL3- | RASQRVNNNYLA | 134 | GASSRAT | 135 | QQYDRSPLT | 136 |
| VL-1 | | | | | | |
| DLL3- | RASQSVNKNYLA | 137 | GASSRAT | 138 | QQYDRSPLT | 139 |
| VL-2 | | | | | | |
| DLL3- | RASQSVSRGYLA | 140 | GASSRAT | 141 | QQYDTSPIT | 142 |
| VL-3 | | | | | | |
| DLL3- | RSSQSLLHSNGYNYLD | 143 | LGSNRAS | 144 | MQALQTPFT | 145 |
| VL-4 | | | | | | |
| DLL3- | RASQSISSYLN | 146 | AASSLQS | 147 | QQSFTTPLT | 148 |
| VL-5 | | | | | | |
| DLL3- | SPGERATLSCK | 149 | YASNRY | 150 | QQDYTSPWT | 151 |
| VL-6 | | | | | | |
| DLL3- | RASQSVSSSYLA | 152 | GASTRAT | 153 | QQYGTSPLT | 154 |
| VL-7 | | | | | | |
| DLL3- | QASQDIRNYLN | 155 | DASNLET | 156 | QHYDNLPLTF | 157 |
| VL-8 | | | | | | |
| DLL3- | RASQGIRNYLG | 158 | AASSLQS | 159 | LQHDSDLRTF | 160 |
| VL-9 | | | | | | |
| DLL3- | TASSSVSSSY | 161 | STSNLAS | 162 | HQYHRSPFTFGAGTKLKIR | 163 |
| VL-10 | | | | | | |
| DLL3- | RASQDISNY | 164 | YTSRLHS | 165 | QQGDMLPWTFGGGTKLEIK | 166 |
| VL-11 | | | | | | |
| DLL3- | SASSSVSY | 167 | DTSKLAS | 168 | QQWTRNPLTFGAGTKLELK | 169 |
| VL-12 | | | | | | |
| DLL3- | KSSQSVLVYSSNQKNY | 170 | WASTRES | 171 | HQYLSSWTFGGGTKLEIK | 172 |
| VL-13 | | | | | | |
| DLL3- | QATQDIVKN | 173 | YAIELAE | 174 | LQFYEFPFTFGAGTKLELK | 175 |
| VL-14 | | | | | | |
| DLL3- | TASSSVSSSY | 176 | STSNLAS | 162 | HQYHRSPFTFGSGTKLEIK | 178 |
| VL-15 | | | | | | |
| DLL3- | KSSQSLSDSDGKTY | 177 | LVSKLDS | 208 | WQGKHFPWTFGGGTKLEIK | 179 |
| VL-16 | | | | | | |
| DLL3- | SASSSVSY | 180 | LTSNLAS | 181 | QQWRSNPFTFGSGTKLEIK | 182 |
| VL-17 | | | | | | |
| DLL3- | RASENIYYN | 183 | TANSLED | 184 | KQAYDVPPTFGGGTKLEIK | 185 |
| VL-18 | | | | | | |
| DLL3- | RASQNIINY | 186 | YTSRLHS | 187 | QQYSERPYTFGGGTKLEIKR | 188 |
| VL-19 | | | | | | |
| DLL3- | KASQDIHKY | 189 | YTSTLQP | 190 | LQYNNLYTFGGGTKLEIKR | 191 |
| VL-20 | | | | | | |
| DLL3- | QATQDIVKN | 192 | YATELAE | 193 | LQFYEFPFTFGAGTKLELK | 194 |
| VL-21 | | | | | | |
| DLL3- | KSSQSLLNSSNQKNY | 195 | FASTRES | 196 | QQHYSIPLTFGAGTKLELK | 197 |
| VL-22 | | | | | | |
| DLL3- | RASQDIKNY | 198 | YTSRVHS | 199 | QQGYTLPFTFGSGTKLE | 200 |
| VL-23 | | | | | | |
| DLL3- | SASSSVSSRY | 201 | STSNLAS | 202 | HQWSNYPLTFGAGTKLELK | 203 |
| VL-24 | | | | | | |
| DLL3- | SASSSVSY | 204 | DSSKLAS | 205 | QQWSSNPLTFGAGTKLELK | 206 |
| VL-25 | | | | | | |
| DLL3- | KSSQSLSDSDGKTY | 207 | LVSKLDS | 208 | WQGKHFPWTFGGGTKLEIK | 209 |
| VL-26 | | | | | | |
| DLL3- | SASSSVSY | 210 | TTSNLAS | 211 | QQRSLYPYTFGGGTKVEIK | 212 |
| VL-27 | | | | | | |
| DLL3- | TASSSVTSSY | 213 | STSNLAS | 214 | HQFHRSPFTFGSGTKLEIK | 215 |
| VL-28 | | | | | | |
| DLL3- | KSTKSLLNSDGFTY | 216 | LVSNRFS | 217 | FQSNYLPLTFGAGTKLELR | 218 |
| VL-29 | | | | | | |
| DLL3- | KASQSVSND | 219 | YASNRYS | 220 | QQDYSSPWTFGGGTKLEIK | 221 |
| VL-30 | | | | | | |
| DLL3- | RASQDISNY | 222 | YTSRLHS | 223 | QQGNTLPYTFGGGTKLEIK | 224 |
| VL-31 | | | | | | |
| DLL3- | ITTPDIDDD | 225 | EGNSLRP | 226 | LQSDNMPFTFGSGTKLEIK | 227 |
| VL-32 | | | | | | |
| DLL3- | SASSSINY | 228 | DTSKLAS | 229 | HQRSTWTFGGGTKLEIK | 230 |
| VL-33 | | | | | | |
| DLL3- | RASQDVINY | 231 | YTSRLHS | 232 | QQYSERPYTFGGGTKLEIKR | 233 |
| VL-34 | | | | | | |
| DLL3- | RSSQNIVHSDRYTY | 234 | GVSNRFS | 235 | FQGTHVPYTFGGGTKLEIK | 236 |
| VL-35 | | | | | | |
| DLL3- | QATQDIVKN | 237 | YATELAE | 238 | LQFYEFPFTFGAGTKLELK | 239 |
| VL-36 | | | | | | |
| DLL3- | KSTKSLLNSDGFTY | 240 | LVSNRFS | 241 | FQSNYLPLTFGAGTKLELR | 242 |
| VL-37 | | | | | | |
| DLL3- | KASQDINSYL | 243 | RANRLVD | 244 | LQYDEFPLTFGAGTKLELK | 245 |
| VL-38 | | | | | | |
| DLL3- | RASQDISNY | 246 | YTSRLHS | 247 | QQGNTLRTFGGGTKLEIK | 248 |
| VL-39 | | | | | | |
| DLL3- | RASQGIRGT | 249 | STSNLNS | 250 | LQRNAYPLTFGAGTKLELK | 251 |
| VL-40 | | | | | | |
| DLL3- | KASQDINSY | 252 | RANRLVD | 253 | LQYDEFPYTFGGGTKLEIKR | 254 |
| VL-41 | | | | | | |
| DLL3- | KASQSVSND | 255 | YASNRYT | 256 | QQDYTSPWTFGGGTKLEIR | 257 |
| VL-42 | | | | | | |
| DLL3- | KASQDVSIF | 258 | SASYRYT | 259 | QQHYGTPFTFGSGTKLKIR | 260 |
| VL-43 | | | | | | |
| DLL3- | RASENIYSY | 261 | NAKTLAE | 262 | QHHYDSPLTFGAGTKLELR | 263 |
| VL-44 | | | | | | |
| DLL3- | KSSQSLLNSSNQKNY | 264 | FASTRES | 265 | QQHYSIPLTFGAGTKLELK | 266 |
| VL-45 | | | | | | |
| DLL3- | KASQDINSF | 267 | RANRLVD | 268 | LQYDEFPYTFGGGTKLEIKR | 269 |
| VL-46 | | | | | | |
| DLL3- | SASSSVSY | 270 | DTSKLAS | 271 | QQWSSNPYTFGGGTKLEIK | 272 |
| VL-47 | | | | | | |
| DLL3- | SVTSSVSY | 273 | LTSNLAS | 274 | QQWRNNPFTFGSGTKVEIK | 275 |
| VL-48 | | | | | | |
| DLL3- | RSSTGAVTTSNY | 276 | GTNNRAP | 277 | GLWYSNHLVFGGGTKLTVL | 278 |
| VL-49 | | | | | | |
| DLL3- | ITSTDIDDD | 279 | EGNTLRP | 280 | LQSDNMPLTFGAGTKLELK | 281 |
| VL-50 | | | | | | |
| DLL3- | RASSSVNY | 282 | YTSNLAP | 283 | QQFTSSPYTFGGGTKLEIKR | 284 |
| VL-51 | | | | | | |
| DLL3- | RASQDIGYS | 285 | ATSSLDS | 286 | LQYASSPWTFGGGTKLEIK | 287 |
| VL-52 | | | | | | |
| DLL3- | KASQDINSY | 288 | RANRLVD | 289 | LQYDEFPFTFGSGTKLEIK | 290 |
| VL-53 | | | | | | |
| DLL3- | KSSQSLLNSRTRKNY | 291 | WASTRES | 292 | KQSYNLYTFGGGTKLKIKR | 293 |
| VL-54 | | | | | | |
| DLL3- | ITSTDIDDD | 294 | EGNTLRP | 295 | LKRDDLPYTFGGGTQVEIKR | 296 |
| VL-55 | | | | | | |
| DLL3- | TASSSVSSSY | 297 | STSNLAS | 298 | HQYNRSPLTFGAGTKLELK | 299 |
| VL-56 | | | | | | |
| DLL3- | KASQDIKKY | 300 | YTSTLEP | 301 | LQYDILWTFGGGTKLEIK | 302 |
| VL-57 | | | | | | |
| DLL3- | SASSSVSSSY | 303 | RTSNLAS | 304 | RQWSGYPWTFGGGTKLEIK | 305 |
| VL-58 | | | | | | |
| DLL3- | TASSSVSSSY | 306 | STSNLAS | 307 | HQYHRSPFTFGSGTKLEIK | 308 |
| VL-59 | | | | | | |
| DLL3- | RASKSVSTSGYSY | 309 | LASNLES | 310 | QHSRELPLTFGAGTKLELK | 311 |
| VL-60 | | | | | | |
| DLL3- | RASSSVSY | 312 | ATSNLAS | 313 | QQWSSNPPTFGAGTKLELK | 314 |
| VL-61 | | | | | | |
| DLL3- | KASQDVGTA | 315 | WASIRHT | 316 | QQYSSYPLTFGAGTKLELK | 317 |
| VL-62 | | | | | | |
| DLL3- | KASQDINSY | 318 | RANRLVD | 319 | LQYDEFPFTFGSGTKLEIK | 320 |
| VL-63 | | | | | | |
| DLL3- | KASQDVNTA | 321 | SASYRYT | 322 | QQHYSSPYTFGGGTKLEIKR | 323 |
| VL-64 | | | | | | |
| DLL3- | RASENIYSY | 324 | NAKTLAE | 325 | QHHYGTPYTFGGGTKLEIKR | 326 |
| VL-65 | | | | | | |
| DLL3- | SASSSVSY | 327 | DTSNLAS | 328 | QEWSGNPLTFGDGTKLELK | 329 |
| VL-66 | | | | | | |
| DLL3- | KASQSVSND | 330 | YASNRYT | 331 | QQDYSSPPTFGGGTKLEIK | 332 |
| VL-67 | | | | | | |
| DLL3- | RASENIYYS | 333 | NANSLED | 334 | KQTYDVPLTFGAGTKLELK | 335 |
| VL-68 | | | | | | |
| DLL3- | KSSQSLLDSDGTTY | 336 | LVSKLDS | 337 | WQGTHFPLTFGAGTKLELK | 338 |
| VL-69 | | | | | | |
| DLL3- | RASSSVSY | 339 | ATSNLAS | 340 | QQWSSNPYTFGGGTKLEIKR | 341 |
| VL-70 | | | | | | |
| DLL3- | KSSQSLLDSDGTTY | 342 | LVSKLDS | 343 | WQGTHFPLTFGAGTKLELK | 344 |
| VL-71 | | | | | | |
| DLL3- | TSSQSLLTSGNQKNY | 345 | WASTRES | 346 | QNDYSLTFGAGTKLELK | 347 |
| VL-72 | | | | | | |
| DLL3- | HVSQNINVW | 348 | KASNLHT | 349 | QQGQSYPFTFGSGTKLEIK | 350 |
| VL-73 | | | | | | |
| DLL3- | KASQSVDYDGDSY | 351 | AASNLES | 352 | QQSNEDPYTFGGGTKLEIKR | 353 |
| VL-74 | | | | | | |
| DLL3- | KSSQSLLYSSTQOKNY | 354 | WASTRES | 355 | QQYYSYPYTFGGGTKLEIKR | 356 |
| VL-75 | | | | | | |
| DLL3- | SASSSVSY | 357 | STSNLAS | 358 | QQRSSYPPTFGGGTKLEIKR | 359 |
| VL-76 | | | | | | |
| DLL3- | KASQNVGTN | 360 | SASYRYS | 361 | QQYNSYPLTFGGGTKLEIK | 362 |
| VL-77 | | | | | | |
| DLL3- | TASSSVSSSY | 363 | STSNLAS | 364 | HQYHRSPFTFGSGTKLEIK | 365 |
| VL-78 | | | | | | |
| DLL3- | LASQTIGTW | 366 | AATSLAD | 367 | QQLYSTPWTFGGGTKLEIK | 368 |
| VL-79 | | | | | | |
| DLL3- | HASQNINVW | 369 | KASILHT | 370 | QQGQSYPYTFGGGTKLEIK | 371 |
| VL-80 | | | | | | |
| DLL3- | HASQNINVW | 372 | KASNLHT | 373 | QQGQSYPFTFGSGTKLEIK | 374 |
| VL-81 | | | | | | |
| DLL3- | LASQTIGTW | 375 | AATSLAD | 376 | QQLYSTPYTFGGGTKLEIKR | 377 |
| VL-82 | | | | | | |
| DLL3- | RASGSIHNY | 378 | NAKTLVD | 379 | QHFWTTPWTFGGGTKLEIK | 380 |
| VL-83 | | | | | | |
| DLL3- | HVSQNINVW | 381 | KASNLHT | 382 | QQGQSYPFTFGSGTKLEIK | 383 |
| VL-84 | | | | | | |
| DLL3- | LASQTIGTW | 384 | AATSLAD | 385 | QQLYSTPWTFGGGTKLEIK | 386 |
| VL-85 | | | | | | |
| DLL3- | KASQSVSND | 387 | CASNRYT | 388 | QQDYSSPLTFGAGTKLELK | 389 |
| VL-86 | | | | | | |
| DLL3- | KASQSVDHAGDSY | 390 | AASNLES | 391 | QQSNEDPYTFGGGTKLEIKR | 392 |
| VL-87 | | | | | | |
| DLL3- | KASQDINRY | 393 | RANRLVD | 394 | LQYDEFPFTFGSGTKLEIK | 395 |
| VL-88 | | | | | | |
| DLL3- | RASGNIHNY | 396 | NAKTLAD | 397 | QHFWSTPWTFGGGTKLEIK | 398 |
| VL-89 | | | | | | |
| DLL3- | SASSSVSY | 399 | STSNLAS | 400 | HQWSSYHTFGGGTKLEIKR | 401 |
| VL-90 | | | | | | |
| DLL3- | LASQTIGTW | 402 | SATSLAD | 403 | QQLYSTPWTFGGGTKLEIK | 404 |
| VL-91 | | | | | | |
| DLL3- | KASQDVNTA | 405 | SASYRYT | 406 | QQHYSSPYTFGGGTKLEIK | 407 |
| VL-92 | | | | | | |
| DLL3- | RASKSVSTSGYSY | 408 | LASNLES | 409 | QHSRELPFTFGGGTKLEIKR | 410 |
| VL-93 | | | | | | |
| DLL3- | KASQDINSY | 411 | RANRLVD | 412 | LQYDEFPFTFGSGTKLEIK | 413 |
| VL-94 | | | | | | |
| DLL3- | KASQDINNY | 414 | RANRLVD | 415 | LQYDEFPYTFGGGTKLEIKR | 416 |
| VL-95 | | | | | | |
| DLL3- | RSSQSIVHSNGNTY | 417 | KVSNRFS | 418 | FQGSHVPLTFGAGTKLELK | 419 |
| VL-96 | | | | | | |
| DLL3- | KASQSVSND | 420 | YASNRYN | 421 | QQDYSSPWTFGGGTKLEIK | 422 |
| VL-97 | | | | | | |
| DLL3- | RASQDINNY | 423 | YTSRLHS | 424 | QQGDTLPWTFGGGTKLEIK | 425 |
| VL-98 | | | | | | |
| DLL3- | SASSSVSY | 426 | DTSNLAS | 427 | QEWSNNPLTFGDGTKLELK | 428 |
| VL-99 | | | | | | |
| DLL3- | HASQNINVW | 429 | KASHLHT | 430 | QQGQSYPFTFGSGTTLEIK | 431 |
| VL-100 | | | | | | |
| DLL3- | MSSQSLLYSSTQKNY | 432 | WASTRES | 433 | QQYYSYPYTFGGGTKLEIKR | 434 |
| VL-101 | | | | | | |
| DLL3- | SASSSVSY | 435 | LTSNLAS | 436 | QQWRSNPFTFGQGTKLEIKR | 437 |
| VL-102 | | | | | | |
| DLL3- | RASENIYYN | 438 | TANSLED | 439 | KQAYDVPPTFGGGTKLEIK | 440 |
| VL-103 | | | | | | |
| DLL3- | SASSSVSY | 441 | DSSKLAS | 442 | QQWSSNPLTFGQGTKLEIK | 443 |
| VL-104 | | | | | | |
| DLL3- | KASQSVSND | 444 | YASNRYS | 445 | QQDYSSPWTFGGGTKVEIK | 446 |
| VL-105 | | | | | | |
| DLL3- | KASQSVSND | 447 | YASNRYT | 448 | QQDYTSPWTFGQGTKLEIKR | 449 |
| VL-106 | | | | | | |

In certain embodiments, the anti-DLL3 binding constructs described herein may be prepared using standard molecular biology techniques with regard to selecting antibodies that have a desired specificity. In some embodiments, the anti-DLL3 binding constructs described herein are produced using recombinant DNA technologies. Procedures for the expression and purification of recombinant proteins are well established in the art.

In some embodiments, the anti-DLL3 binding constructs described herein demonstrate superior functional characteristics compared to other DLL3 binding constructs known in the art. For example, in some embodiments, the anti-DLL3 binding constructs described herein demonstrate superior binding affinity and/or specificity for DLL3, demonstrate a faster on-rate (kₒₙ) and/or a slower off-rate (k_{off}) for DLL3, demonstrate binding to a different or novel epitope of DLL3, demonstrate a longer half-life in vitro and/or in vivo, demonstrate species cross-reactivity with DLL3 from non-human species (e.g., mouse and/or cynomolgus), and/or demonstrate enhanced cytotoxicity of DLL3-expressing cells compared to other DLL3 binding constructs known in the art. In some embodiments, the anti-DLL3 binding constructs described herein demonstrate superior tumor penetration and/or enhanced tumor biodistribution, for example, due to their small sizes and/or binding to human serum albumin (HSA). In some embodiments, the anti-DLL3 binding constructs described herein demonstrate superior manufacturing characteristics compared to other anti-DLL3 constructs known in the art, for example increased protein titer, decreased protein aggregation, and/or increase protein stability.

In some embodiments, the anti-DLL3 binding constructs described herein demonstrate superior binding affinity and/or specificity for human DLL3, as compared to a reference anti-DLL3 construct. In some embodiments, the anti-DLL3 binding constructs described herein bind to human DLL3 with a dissociation constant (K_{D}, M) of less than 1.0E-07, 7.0E-08, 5.0E-08, 3.0E-08, 2.0E-08, 1.0E-08, 7.0E-09, 5.0E-09, 3.0E-09, 2.0E-09, 1.0E-09, 7.0E-10, 5.0E-10, 3.0E-10, 2.0E-10, or 1.0E-10, including all ranges and subranges therebetween. In some embodiments, the anti-DLL3 binding constructs described herein have a K_{D} of less than 1.0E-08 M (i.e., less than 10 nM). In some embodiments, the anti-DLL3 binding constructs described herein have a K_{D} of less than 7.0E-09 M (i.e., less than 7 nM).

In some embodiments, the anti-DLL3 binding constructs described herein demonstrate a faster on-rate (kₒₙ) and/or a slower off-rate (k_{off}) for human DLL3, as compared to a reference anti-DLL3 construct. In some embodiments, the anti-DLL3 binding constructs described herein bind to human DLL3 with an on-rate (kₒₙ, 1/Ms) of at least 5.0E+03, 7.0E+03, 1.0E+04, 2.0E+04, 3.0E+04, 5.0E+04, 7.0E+04, 1.0E+05, 2.0E+05, 3.0E+05, 5.0E+05, 7.0E+05, 1.0E+06, 2.0E+06, 3.0E+06, 5.0E+06, 7.0E+06, or 1.0E+07, including all ranges and subranges therebetween. In some embodiments, the anti-DLL3 binding constructs described herein have an on-rate (kₒₙ 1/Ms) of at least 5.0E+04. In some embodiments, the anti-DLL3 binding constructs described herein have an on-rate (kₒₙ 1/Ms) of at least 7.0E+04. In some embodiments, the anti-DLL3 binding constructs described herein have an on-rate (kₒₙ 1/Ms) of at least 2.0E+05. In some embodiments, the anti-DLL3 binding constructs described herein bind to human DLL3 with an off-rate (k_{off}, 1/s) of less than 5.0E-03, 3.0E-03, 2.0E-03, 1.0E-03, 7.0E-04, 5.0E-04, 3.0E-04, 2.0E-04, 1.0E-04, 7.0E-05, 5.0E-05, 3.0E-05, 2.0E-05, 1.0E-05, 7.0E-06, or 5.0E-06, including all ranges and subranges therebetween. In some embodiments, the anti-DLL3 binding constructs described herein have an off-rate (k_{off}, 1/s) of less than 2.0E-03. In some embodiments, the anti-DLL3 binding constructs described herein have an off-rate (k_{off}, 1/s) of less than 1.0E-03. In some embodiments, the anti-DLL3 binding constructs described herein have an off-rate (k_{off}, 1/s) of less than 7.0E-04.

In some embodiments, the anti-DLL3 binding constructs described herein bind to human DLL3 and demonstrate species cross-reactivity with DLL3 from non-human species (e.g., mouse and/or cynomolgus). In some embodiments, the anti-DLL3 binding constructs described herein demonstrate species cross-reactivity with one or more of mouse, rat, guinea pig, hamster, gerbil, pig, cat, dog, rabbit, goat, sheep, cow, horse, camel, cynomolgus, marmoset, rhesus and chimpanzee DLL3. In some embodiments, the anti-DLL3 binding constructs described herein demonstrate species cross-reactivity with both human and mouse DLL3. In some embodiments, the anti-DLL3 binding constructs described herein demonstrate species cross-reactivity with both human and cynomolgus DLL3. In some embodiments, the anti-DLL3 binding constructs described herein demonstrate species cross-reactivity with human, mouse and cynomolgus DLL3. In some embodiments, species cross-reactivity refers to having similar dissociation constant (K_{D}) values for binding to DLL3 proteins from the two or more species. In some embodiments, the dissociation constant (K_{D}) values are considered “similar” when they are within a 10-fold range, for example, within a 7-fold, 5-fold, 3-fold, or 2-fold range, with one another. In some embodiments, species cross-reactivity refers to having similar EC50 values in the cytotoxicity assay for DLL3 proteins from the two or more species. In some embodiments, the EC50 values in the cytotoxicity assay are considered “similar” when they are within a 10-fold range, for example, within a 7-fold, 5-fold, 3-fold, or 2-fold range, with one another.

In some embodiments, the anti-DLL3 binding constructs described herein demonstrate enhanced cytotoxicity of human DLL3-expressing cells, as compared to a reference anti-DLL3 construct. In some embodiments, the cytotoxicity is mediated at least in part by T cells. In some embodiments, the anti-DLL3 binding constructs described herein have an cytotoxicity EC50 of less than 20 nM, 10 nM, 7 nM, 5 nM, 3 nM, 2 nM, 1 nM, 0.7 nM, 0.5 nM, 0.3 nM, 0.2 nM, 0.1 nM, 0.07 nM, 0.05 nM, 0.03 nM, 0.02 nM, or 0.01 nM, including all ranges and subranges therebetween. In some embodiments, the anti-DLL3 binding constructs described herein have an cytotoxicity EC50 of less than 1 nM. In some embodiments, the anti-DLL3 binding constructs described herein have a cytotoxicity EC50 of less than 0.5 nM.

The binding properties of the anti-DLL3 binding constructs described herein can be characterized using assays known in the art. Non-limiting, exemplary in vitro assays for measuring binding properties are illustrated in Examples herein. For example, the binding affinities and kinetic constants can be determined by a Biacore/SPR instrument using the assay format as defined in Example 4 herein; difference of binding epitopes can be determined by an antibody binning experiment using the assay format as defined in Example 3 herein; the EC50 of cytotoxicity can be determined by a cellular assay according to Example 5 herein.

In some embodiments, the anti-DLL3 binding constructs of the disclosure demonstrate a longer half-life in vitro and/or in vivo. In some embodiments, the anti-DLL3 binding construct comprises a human serum albumin (HSA) binding domain or Fc fusion, and the in vivo half-life of the anti-DLL3 binding construct comprising the HSA binding domain or Fc fusion is extended by about 1-fold, about 2-fold, about 3-fold, about 4-fold, about 5-fold, about 7-fold, about 10-fold, about 15-fold, about 20-fold, about 30-fold, about-40 fold, or about 50-fold compared to the antibody or antigen-binding fragment thereof that does not comprise an HSA binding domain or Fc fusion. In some embodiments, the in vivo half-life of the anti-DLL3 binding construct comprising the HSA binding domain or Fc fusion is extended by 1-fold, 2-fold, 3-fold, 4-fold, 5-fold, 7-fold, 10-fold, 15-fold, 20-fold, 30-fold, about-40 fold, 50-fold, or any value in between, compared to the antibody or antigen-binding fragment thereof that does not comprise an HSA binding domain or Fc fusion. In some embodiments, the in vivo half-life of the anti-DLL3 binding construct comprising the HSA binding domain or Fc fusion is extended by at least 1-fold, at least 2-fold, at least 3-fold, at least 4-fold, at least 5-fold, at least 7-fold, at least 10-fold, at least 15-fold, at least 20-fold, at least 30-fold, about-40 fold, or at least 50-fold compared to the antibody or antigen-binding fragment thereof that does not comprise an HSA binding domain or Fc fusion.

### Polynucleotides and Methods of Protein Expression

The disclosure also includes polynucleotides (e.g., DNA or RNA) encoding the anti-DLL3 binding constructs of the present disclosure. In some embodiments, the polynucleotides encode a polypeptide that is substantially identical to a polypeptide listed in Tables 1, 2, and 3. In some embodiments, the polynucleotides encode a polypeptide that is at least 80%, at least 90%, at least 95%, at least 96%, at least 97%, at least 98%, at least 99%, or 100% identical to a polypeptide listed in Tables 1, 2, and 3. Polynucleotides of the disclosure also include complementary nucleic acids. In some instances, the sequences will be fully complementary (no mismatches) when aligned. In other instances, there can be up to about a 20% mismatch in the sequences. The polynucleotide sequences provided herein can be exploited using codon optimization, degenerate sequence, silent mutations, and other DNA techniques to optimize expression in a particular host, and the present disclosure encompasses such sequence modifications.

In some embodiments, the present disclosure provides an mRNA polynucleotide encoding the anti-DLL3 binding constructs described herein. In some embodiments, the mRNA polynucleotides encode a polypeptide that is substantially identical to a polypeptide listed in Tables 1, 2, and 3. In some embodiments, the mRNA polynucleotides encode a polypeptide that is at least 80%, at least 90%, at least 95%, at least 96%, at least 97%, at least 98%, at least 99%, or 100% identical to a polypeptide listed in Tables 1, 2, and 3.

In some embodiments, the polynucleotides of the present disclosure are inserted into a nucleic acid vector. The nucleic acid vector may be a viral vector or a non-viral vector, e.g. a plasmid. Vectors include, without limitation, plasmids, phagemids, cosmids, transposons, artificial chromosomes such as yeast artificial chromosome (YAC), bacterial artificial chromosome (BAC), or P1-derived artificial chromosome (PAC), bacteriophages such as lambda phage or M13 phage, and animal viruses. In some embodiments, the vector is a plasmid selected from pXT1, pSG5 (Stratagene), pSVK3, pBPV, pMSG, and pSVLSV40 (Pharmacia). In some embodiments, the vector is a viral vector selected from viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., U.S. Pat. No. 7,078,387; Ali et al., Hum Gene Ther 9:81 86, 1998, Flannery et al, PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al, Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Examples of vectors are pClneo vectors (Promega) for expression in mammalian cells; pLenti4/V5-DEST™ pLenti6/V5-DEST™, and pLenti6.2/V5-GW/lacZ (Invitrogen) for lentivirus-mediated gene transfer and expression in mammalian cells.

In some embodiments, the polynucleotide is inserted into a nucleic acid vector and is operably linked to one or more regulatory sequences that control transcription, such as promoters, enhancers, terminators, inducers, or repressors. Exemplary promoters include Non-limiting examples of suitable eukaryotic promoters (promoters functional in a eukaryotic cell) include those from cytomegalovirus (CMV) immediate early, herpes simplex virus (HSV) thymidine kinase, a viral simian virus 40 (SV40) (e.g., early and late SV40), a spleen focus forming virus (SFFV) promoter, long terminal repeats (LTRs) from retrovirus (e.g., a Moloney murine leukemia virus (MoMLV) LTR promoter or a Rous sarcoma virus (RSV) LTR), a herpes simplex virus (HSV) (thymidine kinase) promoter, H5, P7.5, and P11 promoters from vaccinia virus, an elongation factor 1-alpha (EFla) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, dl587rev primer-binding site substituted (MND) promoter, and mouse metallothionein-1.

In some embodiments, the vector is introduced into a host cell for expression of the anti-DLL3 binding construct. Accordingly, proteins for use within the present disclosure can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook and Russell, Molecular Cloning: A Laboratory Manual (3rd ed., Cold Spring Harbor Laboratory Press, Cold Spring Harbor, NY, 2001), and Ausubel et al., Short Protocols in Molecular Biology (4th ed., John Wiley & Sons, 1999).

The gene product encoded by a polynucleotide of the disclosure is expressed in any convenient expression system, including, for example, bacterial, yeast, insect, amphibian, and mammalian systems. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB 11 (Hyclone, Logan, UT); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Virginia. Introduction of the DNA construct can use any convenient method, including, e.g. conjugation, bacterial transformation, calcium-precipitated DNA, electroporation, fusion, transfection, infection with viral vectors, biolistics, and the like.

For example, for recombinant expression of an anti-DLL3 binding construct as described herein, an expression vector will generally include a nucleic acid segment encoding one or more of the amino acid sequences provided in Tables 1, 2, and 3 operably linked to a promoter. The expression vector is introduced to a host cell by conventional techniques, and the host cells are then cultured by conventional techniques to produce the encoded polypeptide(s) to produce the corresponding anti-DLL3 binding constructs.

To direct a recombinant protein into the secretory pathway of a host cell, a secretory signal sequence (also known as a leader sequence) is provided in the expression vector. The secretory signal sequence can be that of the native form of the recombinant protein or can be derived from another secreted protein or synthesized de novo. The secretory signal sequence is operably linked to the polypeptide-encoding DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences can be positioned elsewhere in the DNA sequence of interest (see, e.g., U.S. Pat. Nos. 5,037,743 and 5,143,830).

Cultured mammalian cells are suitable hosts for production of recombinant polypeptides and proteins of the present disclosure (e.g., anti-DLL3 binding constructs). Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, U.S. Pat. Nos. 4,713,339; 4,784,950; 4,579,821; and 4,656,134.

Transformed or transfected host cells to produce the polypeptides and proteins of the present disclosure are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins, and minerals. Media can also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

The anti-DLL3 binding constructs of the present disclosure may be purified by conventional protein purification methods, typically by a combination of chromatographic techniques. See generally Affinity Chromatography: Principles & Methods (Pharmacia LKB Biotechnology, Uppsala, Sweden, 1988); Scopes, Protein Purification: Principles and Practice (Springer-Verlag, New York 1994). Proteins comprising an immunoglobulin Fc region can be purified by affinity chromatography on immobilized protein A or protein G. Additional purification steps, such as gel filtration, can be used to obtain the desired level of purity or to provide for desalting, buffer exchange, and the like.

### Oncolytic Viruses

In some embodiments, the polynucleotides encoding the anti-DLL3 binding constructs are incorporated into an oncolytic virus. Examples of oncolytic viruses are known in the art including, but not limited to, herpes simplex virus (HSV), an adenovirus, a polio virus, a vaccinia virus, a measles virus, a vesicular stomatitis virus, an orthomyxovirus, a parvovirus, a maraba virus, or a picornavirus (e.g., a coxsackievirus or a Seneca Valley virus). In some embodiments, the oncolytic viruses described herein are referred to as oncolytic vectors.

In some embodiments, the anti-DLL3 binding constructs are administered to a subject in combination with an oncolytic virus. In some embodiments, an anti-DLL3 binding construct or a polynucleotide encoding the same is administered to a subject in combination with an oncolytic virus. In some embodiments, the polynucleotide is an mRNA polynucleotide.

### Compositions and Methods of Use

In some embodiments, the present disclosure provides of treating cancer in a subject in need thereof, comprising administering to the subject a therapeutically effective amount of an anti-DLL3 binding construct of the present disclosure. In some embodiments, the methods comprise administering a polynucleotide encoding an anti-DLL3 binding construct a subject in need thereof. In some embodiments, the present disclosure provides of treating cancer in a subject in need thereof, comprising administering to the subject a therapeutically effective amount of an oncolytic virus encoding an anti-DLL3 binding construct. In some embodiments, the present disclosure provides of treating cancer in a subject in need thereof, comprising administering to the subject a therapeutically effective amount of an oncolytic virus or polynucleotide encoding the same in combination with an anti-DLL3 binding construct or polynucleotide encoding the same.

The disclosure also encompasses anti-DLL3 binding constructs, as well as polynucleotides encoding the same, for the manufacture of a medicament for treatment of a cancer in a subject.

In some embodiments, for treatment methods and uses described herein, the anti-DLL3 binding construct is delivered in a manner consistent with conventional methodologies associated with management of the disease or disorder for which treatment is sought. In accordance with the disclosure herein, a therapeutically effective amount of the protein or polypeptide is administered to a subject in need of such treatment for a time and under conditions sufficient to prevent or treat the disease or disorder.

A “subject,” as used herein, includes any animal that exhibits a symptom of a disease, disorder, or condition that can be treated with the recombinant viral vectors, compositions, and methods disclosed herein. Suitable subjects (e.g., subjects) include laboratory animals (such as mouse, rat, rabbit, or guinea pig), farm animals (such as horse or cow), and domestic animals or pets (such as cat or dog). Non-human primates and, preferably, human subjects, are included.

The term “treating” and “treatment” as used herein refers to administering to a subject a therapeutically effective amount of a recombinant virus or composition thereof as described herein so that the subject has an improvement in a disease or condition, or a symptom of the disease or condition. The improvement is any improvement or remediation of the disease or condition, or symptom of the disease or condition. The improvement is an observable or measurable improvement or may be an improvement in the general feeling of well-being of the subject. Thus, one of skill in the art realizes that a treatment may improve the disease condition but may not be a complete cure for the disease. A “prophylactically effective amount” refers to an amount of a virus, a viral stock, or a composition effective to achieve the desired prophylactic result. As used herein, “prophylaxis” can mean complete prevention of the symptoms of a disease, a delay in onset of the symptoms of a disease, or a lessening in the severity of subsequently developed disease symptoms. Typically, but not necessarily, since a prophylactic dose is used in subjects prior to or at an earlier stage of disease, the prophylactically effective amount is less than the therapeutically effective amount.

In prophylactic applications, pharmaceutical compositions or medicants comprising the anti-DLL3 binding construct are administered to a subject susceptible to, or otherwise at risk of, a particular disorder in an amount sufficient to eliminate or reduce the risk or delay the onset of the disorder. In therapeutic applications, compositions or medicants comprising a protein of the present disclosure are administered to a subject suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. In some embodiments, the cancer is selected from non-small cell lung cancer (NSCLC), small cell lung cancer (SCLC), small cell bladder cancer, large cell neuroendocrine carcinoma (LCNEC), castration-resistant small cell neuroendocrine prostate cancer (CRPC-NE), carcinoid (e.g., pulmonary carcinoid), glioblastoma multiforme-IDH mutant (GBM-IDH mutant), Merkel cell carcinoma, and gastric neuroendocrine tumors.

For administration, the anti-DLL3 binding construct, or the oncolytic virus or the polynucleotide encoding the same, may be formulated as a pharmaceutical composition. A pharmaceutical composition may comprise: (i) a DLL3 binding construct; and (ii) a pharmaceutically acceptable carrier, diluent or excipient. A pharmaceutical composition comprising a DLL3 binding construct can be formulated according to known methods to prepare pharmaceutically useful compositions, whereby the therapeutic molecule is combined in a mixture with a pharmaceutically acceptable carrier, diluent, or excipient. A carrier is said to be a “pharmaceutically acceptable carrier” if its administration can be tolerated by a recipient subject. Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising a polypeptide or protein described herein may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition comprising the anti-DLL3 binding construct, or the oncolytic virus or the polynucleotide encoding the same, may be administered to a subject in a therapeutically effective amount. According to the methods of the present disclosure, the anti-DLL3 binding construct, or the oncolytic virus or the polynucleotide encoding the same, can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, intratumoral, and oral routes of administration. For prevention and treatment purposes, an anti-DLL3 binding construct can be administered to a subject in a single bolus delivery, via continuous delivery (e.g., continuous transdermal delivery) over an extended time period, or in a repeated administration protocol (e.g., on an hourly, daily, weekly, or monthly basis).

Effective doses of the compositions of the present disclosure vary depending upon many different factors, including means of administration, target site, physiological state of the subject, whether the subject is human or an animal, other medications administered, whether treatment is prophylactic or therapeutic, as well as the specific activity of the composition itself and its ability to elicit the desired response in the individual. Usually, the subject is a human, but in some diseases, the subject can be a nonhuman mammal. Typically, dosage regimens are adjusted to provide an optimum therapeutic response, i.e., to optimize safety and efficacy.

Determination of effective dosages in this context is typically based on animal model studies followed up by human clinical trials and is guided by determining effective dosages and administration protocols that significantly reduce the occurrence or severity of the subject disorder in model subjects. Accordingly, a “therapeutically effective amount,” as used herein, refers to an amount of a compound is an amount that achieves the desired biologic or therapeutic effect, namely an amount that prevents, reduces or ameliorates one or more symptoms of the enumerated diseases being treated or prevented. For example, the therapeutically effective amount of the antibody, or antigen-binding fragment thereof, will depend on the condition to be treated, the severity and course of the condition, whether the antibody is administered for preventive or therapeutic purposes, previous therapy, the subject's clinical history and response to the antibody, the type of antibody, or antigen-binding fragment thereof, used, and the discretion of the attending physician. The anti-DLL3 binding construct is suitably administered to the patent at one time or over a series of treatments and may be administered to the patent at any time from diagnosis onwards. The anti-DLL3 binding construct nay be administered as the sole treatment or in conjunction with other drugs or therapies (such as an oncolytic virus) useful in treating the condition in question.

In some embodiments, the therapeutically effective amount of the anti-DLL3 binding construct is between about 1 ng/kg body weight/day to about 100 mg/kg body weight/day. In some embodiments, the range of antibody administered is from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 pg/kg body weight/day, 10 pg/kg body weight/day to about 100 pg/kg body weight/day, 10 pg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day. Dosages within this range can be achieved by single or multiple administrations, including, e.g., multiple administrations per day or daily, weekly, bi-weekly, or monthly administrations. The anti-DLL3 binding construct may be administered, as appropriate or indicated, as a single dose by bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

For administration to a human adult subject, the therapeutically effective amount may be administered in doses in the range of 0.0006 mg to 1000 mg per dose, including but not limited to 0.0006 mg per dose, 0.001 mg per dose, 0.003 mg per dose, 0.006 mg per dose, 0.01 mg per dose, 0.03 mg per dose, 0.06 mg per dose, 0.1 mg per dose, 0.3 mg per dose, 0.6 mg per dose, 1 mg per dose, 3 mg per dose, 6 mg per dose, 10 mg per dose, 30 mg per dose, 60 mg per dose, 100 mg per dose, 300 mg per dose, 600 mg per dose and 1000 mg per dose, and multiple, usually consecutive daily doses may be administered in a course of treatment. The anti)-DLL3 antibody or antigen-fragment thereof can be administered at different times of the day. In one embodiment the optimal therapeutic dose can be administered in the evening. In another embodiment the optimal therapeutic dose can be administered in the morning. As expected, the dosage will be dependent on the condition, size, age, and condition of the subject.

Dosage of the pharmaceutical composition comprising the anti-DLL3 binding construct can be varied by the attending clinician to maintain a desired concentration at a target site. Higher or lower concentrations can be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc.

Pharmaceutical compositions comprising the anti-DLL3 binding construct can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition

The disclosure relates to a method of treating cancer in a subject in need thereof, comprising administering a prophylactically effective amount or a therapeutically effective amount of an oncolytic virus, a viral stock, or a composition as described herein to the subject.

In some embodiments, the present disclosure provides methods of treating cancer comprising administration of a therapeutically effective amount of an anti-DLL3 binding construct described herein in combination with one or more additional therapeutic agents. In some embodiments, these two therapeutic agents are administered concurrently or sequentially In concurrent administration, these two therapeutic agents are administered at the same time or within a short time frame (for example, within 10 minutes, within 20 minutes, within 30 minutes, within 1 hour, within 2 hours, within 4 hours, within 6 hours, within 12 hours, or within 24 hours, including all ranges and subranges therebetween). In sequential administration, one of the therapeutic agents is administered before the other, for example, at least 2 hours before, at least 4 hours before, at least 6 hours before, at least 12 hours before, at least 1 day before, at least 2 days before, at least 3 days before, at least 4 days before, at least 5 days before, at least 6 days before, at least 1 week before, at least 2 weeks before, at least 3 weeks before, at least 4 weeks before, at least 1 month before, at least 2 months before, or at least 3 months before, including all ranges and subranges therebetween.

In some embodiments, the additional therapeutic agent is selected from a chemotherapeutic agent, an immune checkpoint inhibitor, an oncolytic virus or polynucleotide encoding the same, and an engineered immune cell comprising an engineered antigen receptor.

Engineered antigen receptors refer to non-naturally occurring antigen-specific receptors such as a chimeric antigen receptors (CARs) or a recombinant T cell receptor (TCRs). In some embodiments, the engineered antigen receptor is a CAR comprising an extracellular antigen binding domain fused via hinge and transmembrane domains to a cytoplasmic domain comprising a signaling domain. In some embodiments, the CAR extracellular domain binds to an antigen expressed by a target cell in an MHC-independent manner leading to activation and proliferation of the engineered immune cell. In some embodiments, the extracellular domain of a CAR recognizes a tag fused to an antibody or antigen-binding fragment thereof. In such embodiments, the antigen-specificity of the CAR is dependent on the antigen-specificity of the labeled antibody, such that a single CAR construct can be used to target multiple different antigens by substituting one antibody for another (See e.g., U.S. Pat. Nos. 9,233,125 and 9,624,279; US Patent Application Publication Nos. 20150238631 and 20180104354). In some embodiments, the extracellular domain of a CAR may comprise an antigen binding fragment derived from an antibody. In some embodiments, the extracellular antigen binding domain is an anti-DLL3 antigen binding construct.

In some embodiments, the present disclosure provides a CAR comprising an extracellular antigen binding domain, a hinge, a transmembrane domain, and a cytoplasmic domain wherein the extracellular antigen binding domain is a binding construct described herein. In some embodiments, the extracellular antigen binding domain is an anti-DLL3 antigen binding construct. In some embodiments, the extracellular antigen binding domain is an anti-DLL3 VHH sdAb described herein.

In some embodiments, the intracellular signaling domain of a CAR may be derived from the TCR complex zeta chain (such as CD3 signaling domains), FcγRIII, FcεRI, or the T-lymphocyte activation domain. In some embodiments, the intracellular signaling domain of a CAR further comprises a costimulatory domain, for example a 4-1BB, CD28, CD40, MyD88, or CD70 domain. In some embodiments, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US 20130287748; WO 2015/142675; WO 2014/055657; and WO 2015/090229, incorporated herein by reference).

CARs specific for a variety of tumor antigens are known in the art, for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J Natl Cancer Inst (2014) 107(1):364), carbonic anhydrase K-specific CARs (Lamers et al., Biochem Soc Trans (2016) 44(3):951-959), FR-a-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL13Ra2-specific CARs (Brown et al., Clin Cacner Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2):154-166), MSLN-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta®) and Tisagenlecleucel (Kymriah®). See also, Li et al., J Hematol and Oncol (2018) 11(22), reviewing clinical trials of tumor-specific CARs.

In some embodiments, the engineered antigen receptor is an engineered TCR. Engineered TCRs comprise TCRa and/or TCRP chains that have been isolated and cloned from T cell populations recognizing a particular target antigen. For example, TCRa and/or TCRP genes (i.e., TRAC and TRBC) can be cloned from T cell populations isolated from individuals with particular malignancies or T cell populations that have been isolated from humanized mice immunized with specific tumor antigens or tumor cells. Engineered TCRs recognize antigen through the same mechanisms as their endogenous counterparts (e.g., by recognition of their cognate antigen presented in the context of major histocompatibility complex (MHC) proteins expressed on the surface of a target cell). This antigen engagement stimulates endogenous signal transduction pathways leading to activation and proliferation of the TCR-engineered cells.

Engineered TCRs specific for tumor antigens are known in the art, for example WT1-specific TCRs (JTCR016, Juno Therapeutics; WT1-TCRc4, described in US Patent Application Publication No. 20160083449), MART-1 specific TCRs (including the DMF4T clone, described in Morgan et al., Science 314 (2006) 126-129); the DMF5T clone, described in Johnson et al., Blood 114 (2009) 535-546); and the ID3T clone, described in van den Berg et al., Mol. Ther. 23 (2015) 1541-1550), gp100-specific TCRs (Johnson et al., Blood 114 (2009) 535-546), CEA-specific TCRs (Parkhurst et al., Mol Ther. 19 (2011) 620-626), NY-ESO and LAGE-1 specific TCRs (1G4T clone, described in Robbins et al., J Clin Oncol 26 (2011) 917-924; Robbins et al., Clin Cancer Res 21 (2015) 1019-1027; and Rapoport et al., Nature Medicine 21 (2015) 914-921), and MAGE-A3-specific TCRs (Morgan et al., J Immunother 36 (2013) 133-151) and Linette et al., Blood 122 (2013) 227-242). (See also, Debets et al., Seminars in Immunology 23 (2016) 10-21).

In some embodiments, the engineered antigen receptor is directed against a target antigen selected from a cluster of differentiation molecule, such as CD3, CD4, CD8, CD16, CD24, CD25, CD33, CD34, CD45, CD64, CD71, CD78, CD80 (also known as B7-1), CD86 (also known as B7-2), CD96, CD 116, CD 117, CD123, CD133, and CD138, CD371 (also known as CLL1); a tumor-associated surface antigen, such as 5T4, BCMA (also known as CD269 and TNFRSF17, UniProt #Q02223), carcinoembryonic antigen (CEA), carbonic anhydrase 9 (CAIX or MN/CAIX), CD19, CD20, CD22, CD30, CD40, disialogangliosides such as GD2, ELF2M, ductal-epithelial mucin, ephrin B2, epithelial cell adhesion molecule (EpCAM), ErbB2 (HER2/neu), FCRL5 (UniProt #Q68SN8), FKBP11 (UniProt #Q9NYL4), glioma-associated antigen, glycosphingolipids, gp36, GPRCSD (UniProt #Q9NZD1), mut hsp70-2, intestinal carboxyl esterase, IGF-I receptor, ITGA8 (UniProt #P53708), KAMP3, LAGE-la, MAGE, mesothelin, neutrophil elastase, NKG2D, Nkp30, NY-ESO-1, PAP, prostase, prostate-carcinoma tumor antigen-1 (PCTA-1), prostate specific antigen (PSA), PSMA, prostein, RAGE-1, ROR1, RU1 (SFMBT1), RU2 (DCDC2), SLAMF7 (UniProt #Q9NQ25), survivin, TAG-72, and telomerase; a major histocompatibility complex (MHC) molecule presenting a tumor-specific peptide epitope; tumor stromal antigens, such as the extra domain A (EDA) and extra domain B (EDB) of fibronectin; the Al domain of tenascin-C(TnC Al) and fibroblast associated protein (FAP); cytokine receptors, such as epidermal growth factor receptor (EGFR), EGFR variant III (EGFRvIII), TFGP-R or components thereof such as endoglin; a major histocompatibility complex (MHC) molecule; a virus-specific surface antigen such as an HIV-specific antigen (such as HIV gp120); an EBV-specific antigen, a CMV-specific antigen, a HPV-specific antigen, a Lassa virus-specific antigen, an Influenza virus-specific antigen as well as any derivate or variant of these surface antigens.

In some embodiments, the immune checkpoint inhibitor is an antibody or an antigen binding fragment thereof. In some embodiments, the immune checkpoint inhibitor binds to PD-1 (e.g., the inhibitor is an anti-PD-1 antibody). Anti-PD1 antibodies are known in the art, for example, Nivolumab, Pembrolizumab, Lambrolizumab, Pidilzumab, Cemiplimab, and AMP-224 (AstraZeneca/MedImmune and GlaxoSmithKline), JTX-4014 by Jounce Therapeutics, Spartalizumab (PDR001, Novartis), Camrelizumab (SHR1210, Jiangsu HengRui Medicine Co., Ltd), Sintilimab (IB1I308, Innovent and Eli Lilly), Tislelizumab (BGB-A317), Toripalimab (JS 001), Dostarlimab (TSR-042, WBP-285, GlaxoSmithKline), INCMGA00012 (MGA012, Incyte and MacroGenics), and AMP-514 (MEDI0680, AstraZeneca). In some embodiments, the immune checkpoint inhibitor binds to PD-L1 (e.g., the inhibitor is an anti-PD-L1 antibody). Anti-PDL1 antibodies are known in the art, for example, MEDI-4736, MPDL3280A, Atezolizumab (Tecentriq, Roche Genentech), Avelumab (Bavencio, Merck Serono and Pfizer), and Durvalumab (Imfinzi, AstraZeneca). In some embodiments, the immune checkpoint inhibitor binds to CTLA4 (e.g., the inhibitor is an anti-CTLA4 antibody). Anti-CTLA4 antibodies are known in the art, for example, ipilumumab, tremelimumab, or any of the antibodies disclosed in WO2014/207063. In some embodiments, the immune checkpoint inhibitor is an anti-TIGIT antibody or fragment thereof. Anti-TIGIT antibodies are known in the art, for example tiragolumab (Roche), EOS-448 (iTeos Therapeutics), Vibostolimab (Merck), Domvanalimab (Arcus, Gilead), BMS-986207 (BMS), Etigilimab (Mereo), COM902 (Compugen), ASP8374 (Astellas), SEA-TGT (Seattle Genetics) BGB-A1217 (BeiGene), IBI-939 (Innovent), and M6223 (EMD Serono).

In some embodiments, the method of treating cancer comprises administering to the subject 1) a therapeutically effective amount of an oncolytic virus or polynucleotide encoding the same; and 2) a therapeutically effective amount of a polypeptide comprising an anti-DLL3 binding construct or polynucleotide encoding the same. In some embodiments, the oncolytic virus or polynucleotide encoding the same is administered before the polypeptide comprising an anti-DLL3 binding construct or polynucleotide encoding the same. In some embodiments, the polypeptide comprising an anti-DLL3 binding construct or polynucleotide encoding the same is administered before the oncolytic virus or polynucleotide encoding the same. In some embodiments, the oncolytic virus is a picornavirus. In some embodiments, the picornavirus is a Seneca Valley Virus (SVV) or a Coxsackie virus (CVA).

In some embodiments, the polypeptide comprising an anti-DLL3 binding construct or polynucleotide encoding the same is administered once. In some embodiments, the polypeptide comprising an anti-DLL3 binding construct or polynucleotide encoding the same is administered multiple times or repeatedly. In some embodiments, the oncolytic virus or polynucleotide encoding the same is administered once. In some embodiments, the oncolytic virus or polynucleotide encoding the same is administered multiple times or repeatedly. In some embodiments, repeated administration (e.g., of a polypeptide comprising an anti-DLL3 antigen-binding fragment) is performed every about 3 hours, 6 hours, 12 hours, 1 day, 2 days, 3 days, 4 days, 5 days, 6 days, a week, 2 weeks, 3 weeks, 4 weeks, 1 month, or longer, including all ranges and subranges therebetween, for a duration of time. In some embodiments, the duration of time is about 12 hours, 1 day, 2 days, 3 days, 4 days, 5 days, 6 days, a week, 2 weeks, 3 weeks, 4 weeks, 1 month, 2 months, 3 months, 4 months, 5 months, 6 months, or longer, including all ranges and subranges therebetween.

“Administration” refers herein to introducing a therapeutic agent, a polypeptide, a vector, an oncolytic virus, a viral stock, a conjugate thereof, or a composition thereof into a subject or contacting a therapeutic agent, a polypeptide, a vector, an oncolytic virus, a viral stock, a conjugate thereof, or a composition thereof with a cell and/or tissue. Administration can occur by injection, irrigation, inhalation, consumption, electro-osmosis, hemodialysis, iontophoresis, and other methods known in the art. The route of administration will vary, naturally, with the location and nature of the disease being treated, and may include, for example auricular, buccal, conjunctival, cutaneous, dental, endocervical, endosinusial, endotracheal, enteral, epidural, interstitial, intra-articular, intra-arterial, intra-abdominal, intraauricular, intrabiliary, intrabronchial, intrabursal, intracavernous, intracerebral, intracisternal, intracorneal, intracronal, intracoronary, intracranial, intradermal, intradiscal, intraductal, intraduodenal, intraduodenal, intradural, intraepicardial, intraepidermal, intraesophageal, intragastric, intragingival, intrahepatic, intraileal, intralesional, intralingual, intraluminal, intralymphatic, intramammary, intramedulleray, intrameningeal, instramuscular, intranasal, intranodal, intraocular, intraomentum, intraovarian, intraperitoneal, intrapericardial, intrapleural, intraprostatic, intrapulmonary, intraruminal, intrasinal, intraspinal, intrasynovial, intratendinous, intratesticular, intratracheal, intrathecal, intrathoracic, intratubular, intratumoral, intratympanic, intrauterine, intraperitoneal, intravascular, intraventricular, intravesical, intravestibular, intravenous, intravitreal, larangeal, nasal, nasogastric, oral, ophthalmic, oropharyngeal, parenteral, percutaneous, periarticular, peridural, perineural, periodontal, respiratory, retrotubular, rectal, spinal, subarachnoid, subconjunctival, subcutaneous, subdermal, subgingival, sublingual, submucosal, subretinal, topical, transdermal, transendocardial, transmucosal, transplacental, trantracheal, transtympanic, ureteral, urethral, and/or vaginal perfusion, lavage, direct injection, and oral administration.

In some embodiments, the polypeptide comprising an anti-DLL3 binding construct, or the polynucleotide encoding the same, is administered systemically. In some embodiments, the polypeptide or polynucleotide encoding the same is administered parenterally. In some embodiments, the polypeptide or polynucleotide encoding the same is administered intravenously. In some embodiments, the polypeptide or polynucleotide encoding the same is administered locally. In some embodiments, the polypeptide or polynucleotide encoding the same is administered intratumorally. In some embodiments, the polypeptide is a trispecific binding construct comprising an anti-DLL3 binding domain of the disclosure.

In some embodiments, the oncolytic virus or polynucleotide encoding the same is administered systemically. In some embodiments, the oncolytic virus or polynucleotide encoding the same is administered parenterally. In some embodiments, the oncolytic virus or polynucleotide encoding the same is administered intravenously. In some embodiments, the oncolytic virus or polynucleotide encoding the same is administered locally. In some embodiments, the oncolytic virus or polynucleotide encoding the same is administered intratumorally.

In some embodiments, the polynucleotide encoding the oncolytic virus (e.g., a RNA molecule encoding SVV viral genome or Coxsackievirus viral genome) is encapsulated in a lipid nanoparticle (LNP). In some embodiments, administering the polynucleotide encoding the oncolytic virus comprises administering the LNP containing the polynucleotide.

In some embodiments, the oncolytic virus is a picornavirus. In some embodiments, the oncolytic virus is a Seneca Valley virus (SVV). In some embodiments, the oncolytic virus is a coxsackievirus. In some embodiments, the coxsackievirus is a coxsackievirus A type (CVA)—for example, CVA21. In some embodiments, the oncolytic virus is a herpes simplex virus.

### FURTHER NUMBERED EMBODIMENTS

Further numbered embodiments of the present disclosure are provided as follows:

Embodiment 1: A single domain antibody (sdAb) comprising a complementarity determining region (CDR) 1, a CDR2, and a CDR3, wherein: the CDR1 comprises an amino acid sequence selected from SEQ ID NOs: 1, 6, 10, 14, 18, 22, 26, 30, 33, 36, 39, 46, 52, 60, 65, 69, 73, 77, and 83; the CDR2 comprises an amino acid sequence selected from SEQ ID NOs: 2, 7, 11, 15, 19, 23, 27, 40, 43, 47, 53, 61, 66, 74, 78, 81, and 82; and the CDR3 comprises an amino acid sequence selected from SEQ ID NOs: 3, 8, 12, 16, 20, 24, 28, 31, 34, 37, 41, 44, 48, 54, 62, 67, 71, 75, 79, and 84.

Embodiment 2: The sdAb of Embodiment 1, wherein: the CDR1 comprises an amino acid sequence of SEQ ID NO: 46; the CDR2 comprises an amino acid sequence of SEQ ID NO: 47; and the CDR3 comprises an amino acid sequence of SEQ ID NO: 48.

Embodiment 3: The sdAb of Embodiment 1, wherein: the CDR1 comprises an amino acid sequence of SEQ ID NO: 52; the CDR2 comprises an amino acid sequence of SEQ ID NO: 53; and the CDR3 comprises an amino acid sequence of SEQ ID NO: 54.

Embodiment 4: The sdAb of Embodiment 1, wherein: the CDR1 comprises an amino acid sequence of SEQ ID NO: 18; the CDR2 comprises an amino acid sequence of SEQ ID NO: 19; and the CDR3 comprises an amino acid sequence of SEQ ID NO: 20.

Embodiment 5: The sdAb of Embodiment 1, wherein: the CDR1 comprises an amino acid sequence of SEQ ID NO: 83; the CDR2 comprises an amino acid sequence of SEQ ID NO: 2; and the CDR3 comprises an amino acid sequence of SEQ ID NO: 84.

Embodiment 6: The sdAb of Embodiment 1, wherein: the CDR1 comprises an amino acid sequence of SEQ ID NO: 60; the CDR2 comprises an amino acid sequence of SEQ ID NO: 61; and the CDR3 comprises an amino acid sequence of SEQ ID NO: 62.

Embodiment 7: The sdAb of Embodiment 1, wherein: the CDR1 comprises an amino acid sequence of SEQ ID NO: 65; the CDR2 comprises an amino acid sequence of SEQ ID NO: 66; and the CDR3 comprises an amino acid sequence of SEQ ID NO: 67.

Embodiment 8: The sdAb of Embodiment 1, wherein: the CDR1 comprises an amino acid sequence of SEQ ID NO: 69; the CDR2 comprises an amino acid sequence of SEQ ID NO: 23; and the CDR3 comprises an amino acid sequence of SEQ ID NO: 24.

Embodiment 9: The sdAb of Embodiment 1, wherein: the CDR1 comprises an amino acid sequence of SEQ ID NO: 14; the CDR2 comprises an amino acid sequence of SEQ ID NO: 43; and the CDR3 comprises an amino acid sequence of SEQ ID NO: 71.

Embodiment 10: The sdAb of Embodiment 1, wherein: the CDR1 comprises an amino acid sequence of SEQ ID NO: 73; the CDR2 comprises an amino acid sequence of SEQ ID NO: 74; and the CDR3 comprises an amino acid sequence of SEQ ID NO: 75.

Embodiment 11. The sdAb of Embodiment 1 comprising an amino acid sequence that is at least 95%, at least 96%, at least 97%, at least 98%, or at least 99% identical to an amino acid sequence selected from SEQ ID NOs: 4, 5, 9, 13, 17, 21, 25, 29, 32, 35, 38, 42, 45, 49, 55, 59, 63, 64, 68, 70, 72, and 76.

Embodiment 12. The sdAb of Embodiment 1 comprising or consisting of an amino acid sequence selected from SEQ ID NOs: 4, 5, 9, 13, 17, 21, 25, 29, 32, 35, 38, 42, 45, 49, 55, 59, 63, 64, 68, 70, 72, and 76.

Embodiment 13. A single domain antibody sdAb comprising (a) human framework region sequence and (b) a complementarity determining region (CDR) 1, a CDR2, and a CDR3, wherein (i) the CDR1 comprises an amino acid sequence selected from SEQ ID NOs: 1, 6, 10, 14, 18, 22, 26, 30, 33, 36, 39, 46, 52, 60, 65, 69, 73, 77, and 83; (ii) the CDR2 comprises an amino acid sequence selected from SEQ ID NOs: 2, 7, 11, 15, 19, 23, 27, 40, 43, 47, 53, 61, 66, 74, 78, 81, and 82; and (iii) the CDR3 comprises an amino acid sequence selected from SEQ ID NOs: 3, 8, 12, 16, 20, 24, 28, 31, 34, 37, 41, 44, 48, 54, 62, 67, 71, 75, 79, and 84.

Embodiment 14: The sdAb of Embodiment 13, wherein: the CDR1 comprises an amino acid sequence of SEQ ID NO: 46; the CDR2 comprises an amino acid sequence of SEQ ID NO: 47; and the CDR3 comprises an amino acid sequence of SEQ ID NO: 48.

Embodiment 15: The sdAb of Embodiment 13, wherein: the CDR1 comprises an amino acid sequence of SEQ ID NO: 52; the CDR2 comprises an amino acid sequence of SEQ ID NO: 53; and the CDR3 comprises an amino acid sequence of SEQ ID NO: 54.

Embodiment 16: The sdAb of Embodiment 13, wherein: the CDR1 comprises an amino acid sequence of SEQ ID NO: 18; the CDR2 comprises an amino acid sequence of SEQ ID NO: 19; and the CDR3 comprises an amino acid sequence of SEQ ID NO: 20.

Embodiment 17: The sdAb of Embodiment 13, wherein: the CDR1 comprises an amino acid sequence of SEQ ID NO: 83; the CDR2 comprises an amino acid sequence of SEQ ID NO: 2; and the CDR3 comprises an amino acid sequence of SEQ ID NO: 84.

Embodiment 18: The sdAb of Embodiment 13, wherein: the CDR1 comprises an amino acid sequence of SEQ ID NO: 60; the CDR2 comprises an amino acid sequence of SEQ ID NO: 61; and the CDR3 comprises an amino acid sequence of SEQ ID NO: 62.

Embodiment 19: The sdAb of Embodiment 13, wherein: the CDR1 comprises an amino acid sequence of SEQ ID NO: 65; the CDR2 comprises an amino acid sequence of SEQ ID NO: 66; and the CDR3 comprises an amino acid sequence of SEQ ID NO: 67.

Embodiment 20: The sdAb of Embodiment 13, wherein: the CDR1 comprises an amino acid sequence of SEQ ID NO: 69; the CDR2 comprises an amino acid sequence of SEQ ID NO: 23; and the CDR3 comprises an amino acid sequence of SEQ ID NO: 24.

Embodiment 21: The sdAb of Embodiment 13, wherein: the HCDR1 comprises an amino acid sequence of SEQ ID NO: 14; the HCDR2 comprises an amino acid sequence of SEQ ID NO: 43; and the HCDR3 comprises an amino acid sequence of SEQ ID NO: 71.

Embodiment 22: The sdAb of Embodiment 13, wherein: the CDR1 comprises an amino acid sequence of SEQ ID NO: 73; the CDR2 comprises an amino acid sequence of SEQ ID NO: 74; and the CDR3 comprises an amino acid sequence of SEQ ID NO: 75.

Embodiment 23: The sdAb of Embodiment 13, comprising an amino acid sequence that is at least 95%, at least 96%, at least 97%, at least 98%, or at least 99% identical to an amino acid sequence selected from SEQ ID NOs: 50, 51, 56, 57 and 58.

Embodiment 24: The sdAb of Embodiment 13, comprising or consisting of an amino acid sequence selected from SEQ ID NOs: 50, 51, 56, 57 and 58.

Embodiment 25: The sdAb of any one of Embodiments 1-24, specifically binding to a Delta-like ligand 3 (DLL3) protein.

Embodiment 26: The sdAb of Embodiment 25, wherein the DLL3 protein is a human DLL3 protein.

Embodiment 27: A chimeric antigen receptor (CAR) comprising an extracellular antigen binding domain, a hinge, a transmembrane domain, and a cytoplasmic domain wherein the extracellular antigen binding domain is the sdAb of any one of Embodiments 1-26.

Embodiment 28: A biomolecule comprising the sdAb of any one of Embodiments 1-26.

Embodiment 29: A polynucleotide encoding the sdAb of any one of Embodiments 1-26, the CAR of Embodiment 27, or the biomolecule of Embodiment 28.

Embodiment 30: The polynucleotide of Embodiment 29, wherein the polynucleotide is an mRNA molecule.

Embodiment 31: A vector comprising the polynucleotide of Embodiment 29 or Embodiment 30.

Embodiment 32: A binding construct comprising the sdAb of any one of Embodiments 1-26.

Embodiment 33: A binding construct comprising the sdAb of any one of Embodiments 1-26 and an Fc domain.

Embodiment 34: A bispecific binding construct comprising a first binding domain and a second binding domain, wherein the first binding domain comprises the sdAb of any one of Embodiments 1-26; and wherein the second binding domain is selected from a antigen binding domain that specifically binds to CD3, CD16 or NKp46, or human serum albumin (HSA), or an Fc domain.

Embodiment 35: A trispecific binding construct comprising a first binding domain, a second binding domain and a third binding domain, wherein the first binding domain comprises the sdAb of any one of Embodiments 1-26; wherein the second binding domain comprises an Fc domain or an antigen binding domain that specifically binds to human serum albumin (HSA); and wherein the third binding domain is an antigen binding domain specifically binds to CD3, CD16 or NKp46.

Embodiment 36: The trispecific binding construct of Embodiment 35, wherein the second binding domain is an antigen binding domain that specifically binds to HSA, and the third binding domain is an antigen binding domain that specifically binds to CD3.

Embodiment 37: A polynucleotide encoding the binding construct of Embodiment 32 or 33, the bispecific binding construct of Embodiment 34, or the trispecific binding construct of any one of Embodiments 35-36.

Embodiment 38: The polynucleotide of Embodiment 37, wherein the polynucleotide is an mRNA molecule.

Embodiment 39: A vector comprising the polynucleotide of Embodiment 37.

Embodiment 40: A method of treating cancer in a subject in need thereof, comprising administering the sdAb of any one of Embodiments 1-26, the CAR of Embodiment 27, the biomolecule of Embodiment 28, the binding construct of Embodiment 32 or 33, the bispecific binding construct of Embodiment 34, or the trispecific binding construct of any one of Embodiments 35-36 to the subject.

Embodiment 41: Use of the sdAb of any one of Embodiments 1-26, the CAR of Embodiment 27, the biomolecule of Embodiment 28, the binding construct of Embodiment 32 or 33, the bispecific binding construct of Embodiment 34, or the trispecific binding construct of any one of Embodiments 35-36, for treating cancer in a subject in need thereof.

Embodiment 42: Use of the sdAb of any one of Embodiments 1-26, the CAR of Embodiment 27, the biomolecule of Embodiment 28, the binding construct of Embodiment 32 or 33, the bispecific binding construct of Embodiment 34, or the trispecific binding construct of any one of Embodiments 35-36 in the manufacture of a medicament for treating cancer in a subject in need thereof.

Embodiment 43: The method of Embodiment 40 or use of Embodiment 41 or 42, wherein the cancer is selected from non-small cell lung cancer (NSCLC), small cell lung cancer (SCLC), small cell bladder cancer, large cell neuroendocrine carcinoma (LCNEC), castration-resistant small cell neuroendocrine prostate cancer (CRPC-NE), carcinoid (e.g., pulmonary carcinoid), glioblastoma multiforme-IDH mutant (GBM-IDH mutant), Merkel cell carcinoma, and Gastric neuroendocrine cancer.

Embodiment 44: The method of Embodiment 40 or 43, or the use of any one of Embodiments 41-43, wherein the sdAb or the binding construct is administered in combination with an oncolytic virus or a polynucleotide encoding the oncolytic virus.

Embodiment 45: The method or use of Embodiment 44, wherein the sdAb or the binding construct, and the oncolytic virus or the polynucleotide encoding the oncolytic virus, are administered concurrently.

Embodiment 46: The method or use of Embodiment 44, wherein the sdAb or the binding construct, and the oncolytic virus or the polynucleotide encoding the oncolytic virus, are administered sequentially.

Embodiment 47: The method or use of any one of Embodiments 44-46, wherein the oncolytic virus is a picornavirus.

Embodiment 48: The method or use of Embodiment 47, wherein the picornavirus is a Coxsackievirus.

Embodiment 49: The method or use of Embodiment 47, wherein the picornavirus is a Seneca Valley virus.

Embodiment 50: The method or use of any one of Embodiments 44-49, wherein the polynucleotide encoding the oncolytic virus is encapsulated in a lipid nanoparticle.

Embodiment 51: An oncolytic virus expressing one or more payload molecules, wherein the one or more payload molecules comprise the sdAb of any one of Embodiments 1-26, the binding construct of Embodiment 32 or 33, the bispecific binding construct of Embodiment 34 or 34.b, or the trispecific binding construct of any one of Embodiments 35-36.

Embodiment 52: The oncolytic virus of Embodiment 51, wherein the virus is selected from a herpes simplex virus, an adenovirus, a polio virus, a vaccinia virus, a measles virus, a vesicular stomatitis virus, an orthomyxovirus, a parvovirus, a maraba virus, a picornavirus, a togaviriadae virus, a semliki forest virus, a sindbis virus, a paramyxoviridae virus, and a sendai virus.

Embodiment 53: The oncolytic virus of Embodiment 52, wherein the picornavirus is a coxsackievirus or a seneca valley virus.

Embodiment 54: A method of treating cancer in a subject in need thereof, comprising administering the oncolytic virus of any one of Embodiments 51-53 to the subject.

Embodiment 55: Use of the oncolytic virus of any one of Embodiments 51-53 for treating cancer in a subject in need thereof.

Embodiment 56: Use of the oncolytic virus of any one of Embodiments 51-53 in the manufacture of a medicament for treating cancer in a subject in need thereof.

Embodiment 57: A messenger RNA (mRNA) polynucleotide encoding the sdAb of any one of Embodiments 1-26, the binding construct of Embodiment 32 or 33, the bispecific binding construct of Embodiment 34 or 34.b, or the trispecific binding construct of any one of Embodiments 35-36.

Embodiment 58: A method of treating cancer in a subject in need thereof, comprising administering the mRNA polynucleotide of Embodiment 57 to the subject.

Embodiment 59: Use of the mRNA polynucleotide of Embodiment 57 for treating cancer in a subject in need thereof.

Embodiment 60: Use of the mRNA polynucleotide of Embodiment 57 in the manufacture of a medicament for treating cancer in a subject in need thereof.

Embodiment 61: The method of Embodiment 58 or the use of Embodiment 59 or 60, wherein the mRNA polynucleotide is administered in combination with an oncolytic virus.

Embodiment 62: The method of Embodiment 58 or the use of Embodiment 59 or 60, wherein the mRNA polynucleotide is administered in combination with a polynucleotide encoding an oncolytic virus.

### Examples

### Example 1: Cytotoxicity of Anti-DLL3/Anti-CD3 Bispecific Binding Constructs

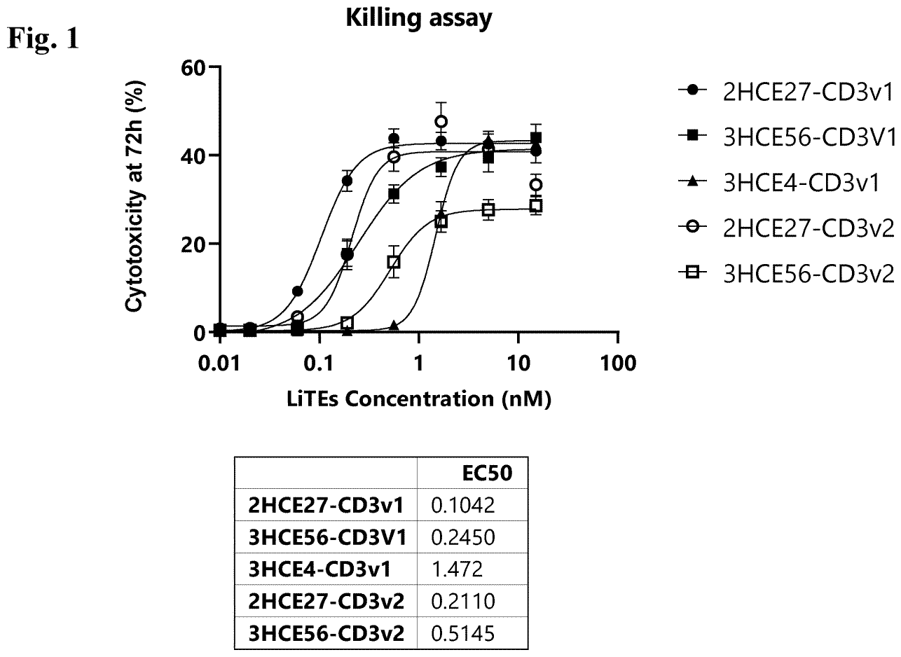

Experiments were performed to assess the cytotoxicity of multiple anti-DLL3/anti-CD3 bispecific binding constructs. The bispecific binding constructs used in this example comprised an anti-DLL3 VHH domain linked to an anti-CD3 scFv (also referred to herein as a LiTE construct). The anti-DLL3 VHH domains used are the 2HCE27 (SEQ ID NO: 49), 3HCE56 (SEQ ID NO: 55), and 3HCE4 (SEQ ID NO: 21). Human CD3 T cells were incubated with CHO cells expressing human DLL3 at an effector-to-target (E:T) of 10:1. Different dilutions of each LiTEs were tested as indicated in FIG. 1. The Caspase 3/7 area/Phase area % was measured according to the IncuCyte® immune cell killing assay protocol (Essen Bioscience) at 72 hours to determine the cytotoxicity % and the EC50 of each LiTE was determined. Results are shown in FIG. 1. All constructs display cytotoxicity. Among all the VHH clones, 2HCE27-CD3v1 displayed the best activity in the killing assay. CD3v1 was better than CD3v2 at induce the killing of the target cells when combined with 2HCE27 VHH.

### Example 2: Cross-Reactivity of Anti-DLL3/Anti-CD3 Bispecific Binding Constructs

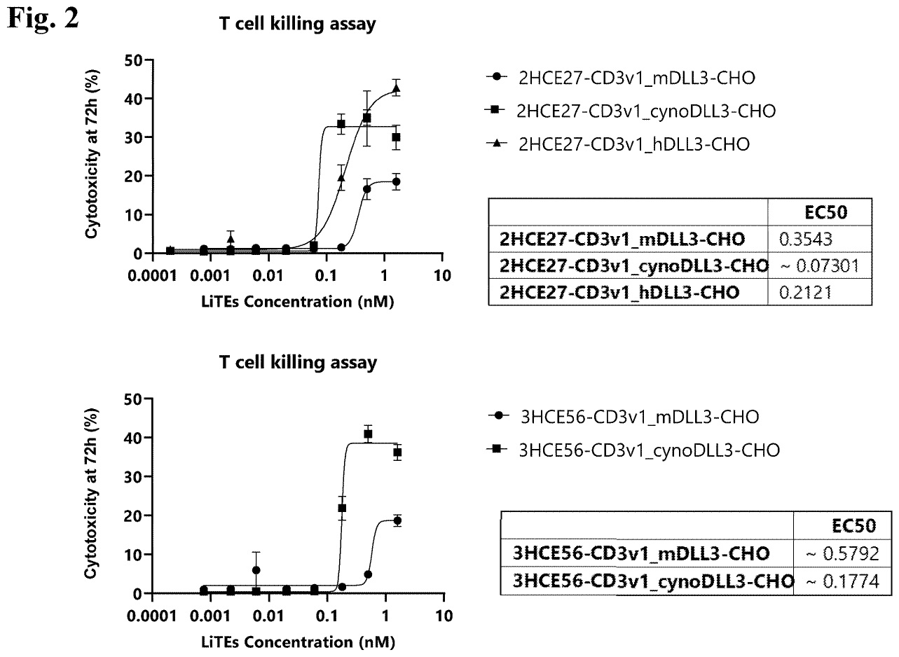

The 2HCE27 and 3HCE56 LiTE constructs described in Example 1 were tested in an in vitro assay to determine the ability of each to bind to murine, human, or cynomolgus DLL3. Human CD3 T cells were incubated with CHO cells expressing either human DLL3 or mouse DLL3 or cyno DLL3 at an E:T 10:1. Different dilution of LiTEs were tested. At 72 hours the Caspase 3/7 area/Phase area % was measured according to the IncuCyte® immune cell killing assay protocol (Essen Bioscience) to reflect the cytotoxicity %. Results are shown in FIG. 2. Both 2HCE27 and 3HCE56 LiTEs induced killing of target cells expressing cyno or mouse DLL3, demonstrating that both anti-human VHHs can cross-react with cyno and mouse DLL3.

### Example 3: Binding Properties of Anti-DLL3 Constructs

Antibody binning experiments were carried out for 5 anti-DLL3 VHHs and an anti-human DLL3 BiTE (SEQ ID NO: 756). The target antigen was coated on a series of Octet SA tips. Each series of tips was first soaked in a periplasmic extract containing one specific VHH. After the response signals reached saturation, the tips were transferred to a mix of PEs containing again the first VHH (at the same concentration) plus a different VHH (termed secondary VHH) for each tip of the series. Secondary VHH that still bind the target antigen in the presence of the first VHH will show an increased response, as compared to the binding response of the first VHH alone. This was repeated for each VHH as primary VHH until all VHH were tested against each other in both directions (as primary and as secondary binder). From the response data, the bins were deduced for each VHH. Results are shown in Table 9 below. 2HCE117 and 3HCE4 show competition with the human DLL3 BiTEs, whereas 3HCE56 and 3HCE87 show a partial competition. 2HCE27 does not compete with the human DLL3 BiTE, suggesting that their binding epitopes on DLL3 are different.

TABLE 9
Binning of Anti-DLL3 Antibodies

| | | | BIN 6 | BIN 5 | | |
|---|---|---|---|---|---|---|
| | DLL3 BiTEs | 2HCE27 | 3HCE4 | 3HCE56 | 3HCE87 | 2HCE117 |
| DLL3 BiTEs | 0.00 | 82.52 | 121.36 | 1501.59 | 125.35 | 1279.87 |
| 2HCE27 | 83.38 | 0.00 | 53.42 | 6495.12 | 87.49 | 19919.16 |
| 3HCE4 | 9.03 | 346.55 | 0 | 406.99 | 120.36 | 3507.51 |
| 3HCE56 | 44.06 | −3.58 | −79.59 | 0.00 | −99.14 | 1114.98 |
| 3HCE87 | 68.35 | −48.19 | −54.40 | 203.52 | 0.00 | 1210.99 |
| 2HCE117 | −23.17 | −73.01 | −90.13 | −206.97 | −98.70 | 0.00 |

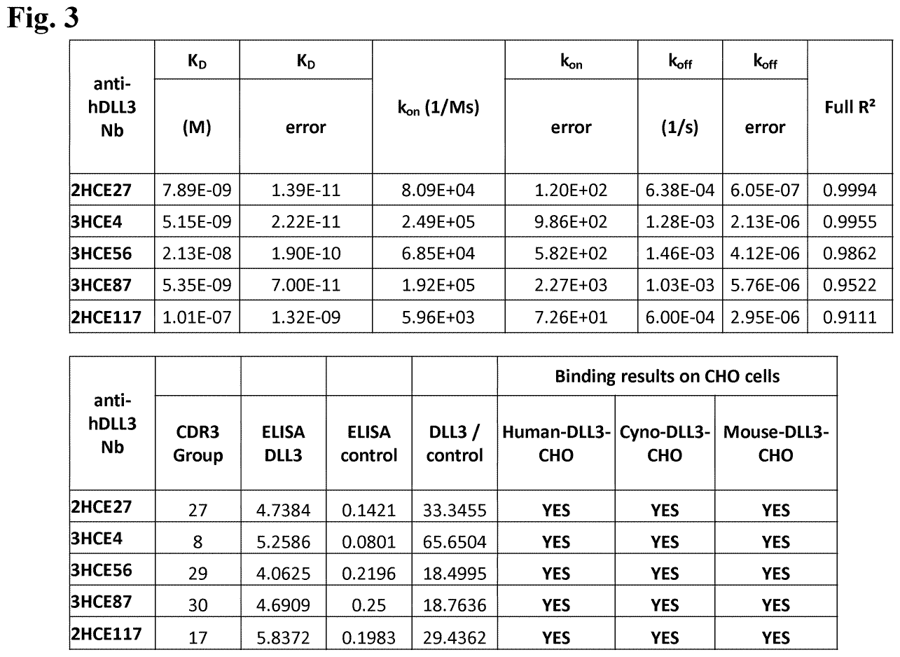

Additional experiments were carried out to further characterize the binding properties of the anti-DLL3 constructs. Binding properties tested include dissociation constant (K_{D}), off-rate (k_{off}), binding to DLL3 protein by ELISA and binding to CHO cells expressing human, cyno or mouse DLL3. To obtain off-rate (k_{off}), human biotinylated DLL3 was immobilized on Octet SA tips, and cynomolgus or mouse DLL3 was immobilized on Octet AR2G tips. For each DLL3 protein, the binding to a dilution series of each anti-DLL3 construct was measured using Fortebio Octet Red. The dissociation constant (K_{D}), off-rate (k_{off}) and other kinetic parameters for each anti-DLL3 construct were calculated using a 1:1 binding model. In addition, binding of these anti-DLL3 constructs to CHO cells expressing human, cyno or mouse DLL3 protein was analyzed using flow cytometry (FACS). Results are shown in FIG. 3, which demonstrates that all anti-DLL3 constructs tested (2HCE27, 3HCE4, 3HCE56, 3HCE87 and 2HCE117) have high affinity binding to human DLL3 protein, as well as binding to cyno and mouse DLL3 proteins.

### Example 4: Binding and Cross-Reactivity of an Anti-DLL3/Anti-CD3/Anti-HSA Trispecific Binding Construct

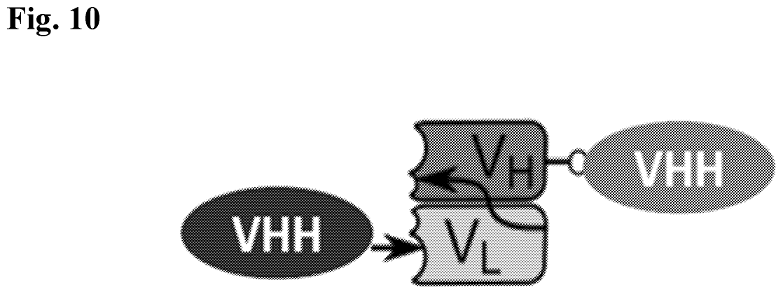

Experiments were performed to assess the binding and cross-reactivity of an anti-DLL3/anti-CD3/anti-HSA trispecific binding construct. The domain organization of the trispecific binding construct is shown in FIG. 10. The anti-DLL3 VHH domain in the trispecific binding construct is 2HCE27 (SEQ ID NO: 49).

Surface Plasmon Resonance (SPR) was used to assess the binding kinetics and binding affinity. Kinetic rate coefficients were recovered from binding analysis experiments performed with a Biacore 3000 biosensor. Concentrations of the anti-DLL3/anti-CD3/anti-HSA trispecific binding construct ranging from 1.23 nM to 300 nM were run against the SPR sensorgram surfaces immobilized with recombinant human CD3 (rhu CD3) or recombinant HSA (rHSA). Concentrations of recombinant human DLL3 (rhu DLL3) ranging from 1.23 nM to 100 nM were run against the SPR sensorgram surface immobilized with the anti-DLL3/anti-CD3/anti-HSA trispecific binding construct. The association and dissociation phase data were globally fit to a 1:1 model to determine the association rate coefficient (kₒₙ), dissociation rate coefficient (k_{off}), the Rmax value, and the dissociation constant (K_{D}). Results were reported as the global fits to 1:1 model±standard error. The control groups were the three corresponding individual binding constructs. The results are shown in Table 10 below. All three antigen binding domains in the trispecific binding construct retain binding to the corresponding antigen. The anti-DLL3 binding domain in the trispecific binding construct shows comparable dissociation constant (K_{D}) as the isolated anti-DLL3 binding construct.

TABLE 10
Comparison of Binding Properties of the anti-DLL3/anti-CD3/anti-
HSA trispecific Binding Construct with Corresponding Individual
Binding Constructs

| | | kₒₙ 1 × | k_{off} 1 × | | |
|---|---|---|---|---|---|
| Binding | | 10⁴ | 10⁻³ | Rmax | K_{D} |
| Construct | Ligand | (M⁻¹s⁻¹) | (s⁻¹) | (RU) | (nM) |
| anti-DLL3/ | rhu | 33.67 ± 0.03 | 0.39 ± | 50.18 ± 0.01 | 1.15 ± |
| anti-CD3/ | CD3 | | 0.01 | | 0.01 |
| anti-HSA | rHSA | 2.7 ± 0.01 | 1.75 ± | 83.73 ± 0.07 | 64.7 ± 0.1 |
| trispecific | | | 0.01 | | |
| binding | rhu | 9.84 ± 0.02 | 0.64 ± | 43.27 ± 0.04 | 6.47 ± |
| construct | DLL3 | | 0.001 | | 0.01 |
| anti-CD3 | rhu | 510 | 0.57 | 11.1 | 0.112 |
| | CD3 | | | | |
| anti-HSA | rHSA | | | | 0.91 |
| anti-DLL3 | rhu | 8 ± 0.01 | 0.64 ± | | 7.89 ± |
| (2HCE27) | DLL3 | | 0.001 | | 0.01 |

Cell binding assays were performed to determine the ability of the anti-DLL3/anti-CD3/anti-HSA trispecific binding construct to bind to human or cynomolgus (cyno) DLL3 and CD3.

5×10⁴ CHO or cancer cells/well were plated in a 96 well round plate. Cells were incubated for 30 min at 4° C. with 0.02 mL block solution (10% goat+10% rabbit serum diluted in Flow media (PBS, 1% BSA)) and then washed. The trispecific binding construct was titrated from 120 nM to 0.1 nM in serial 3-fold dilutions in Flow media, added to the cells at a volume of 30 μL/well, and incubated for 30 min at 4° C. The cells were then washed in Flow media and stained with an anti-His APC conjugated antibody (cat #IC050A, R&D systems) for 20 min at 4° C. to detect the His-tagged trispecific binding construct.

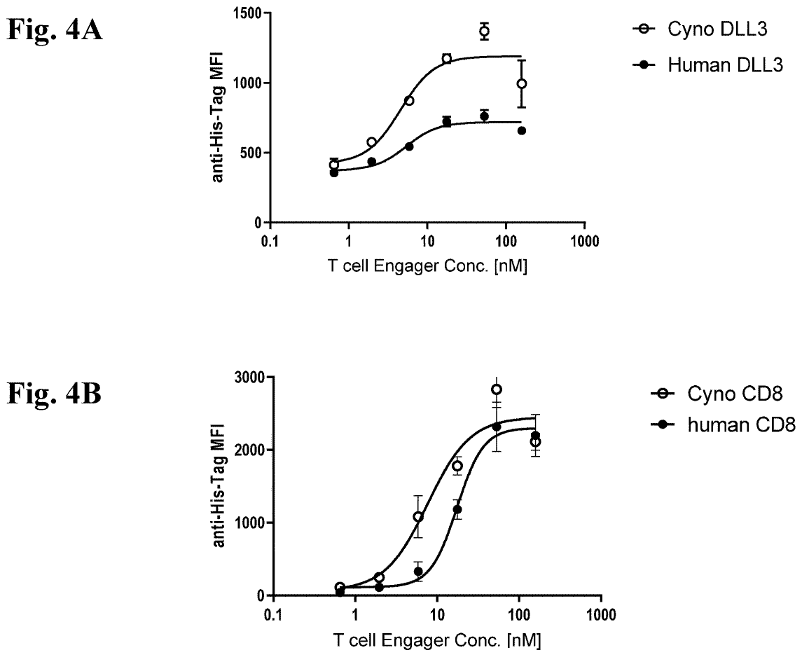

Flow cytometry was used to analyze the presence of the trispecific binding construct on cell surface. CHO cells expressing either human DLL3 or cynomolgus DLL3 were washed 2×0.2 mL with Flow media and then 100 μL Flow media was added. 5 μL 7AAD was added 5 min before plate analyzing to enable gating of live versus dead cells. Data were acquired on a BD LSRFortessa using BD FACSDiva software, and analyzed using FlowJo software. The EC50 values of the trispecific binding construct were calculated by measuring the APC MFI in the live cells gate. The results (FIG. 4A) show that the trispecific binding construct binds to human DLL3 and cynomolgus DLL3 on the cell surface with similar EC50.

For the CD8 human and cyno T cell binding assays, the procedure was similar, but the staining with the anti-His APC conjugated antibody was done in presence of anti-human CD8-PE (clone SK1, Cat #12-087-41, eBioscience), anti-human CD14-BV421 (clone HCD14, Cat #325628, Biolegend), and anti-human CD4-BV605 (clone RPA-T4, Cat #562659BD, Biosciences). The EC50 values of the trispecific binding construct were calculated by measuring the APC MFI in the live cells CD8+ gate. The results (FIG. 4B) show that the trispecific binding construct binds to human CD3 and cynomolgus CD3 on the cell surface with similar EC50.

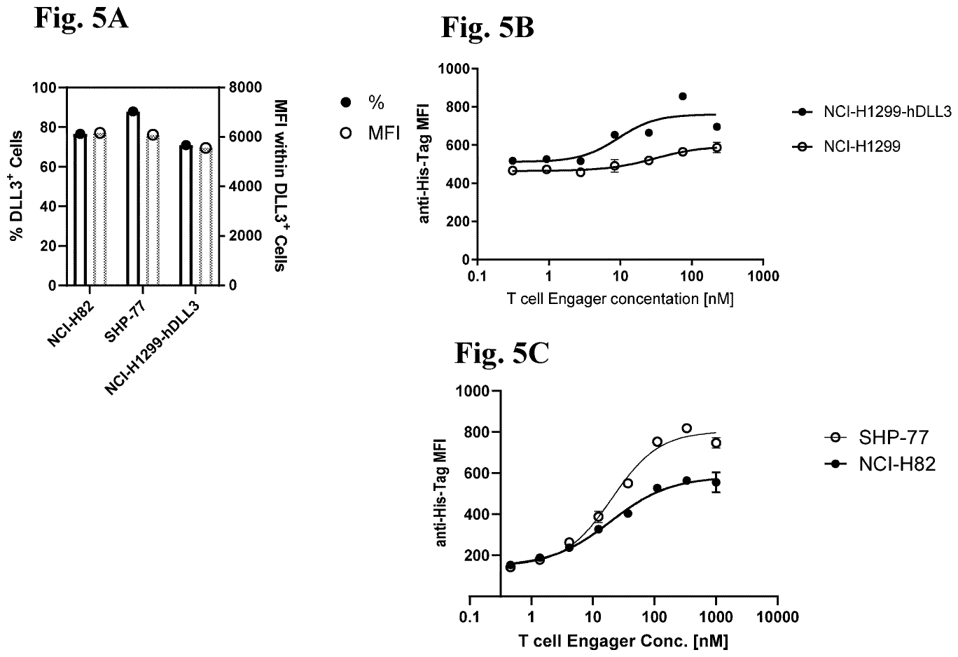

Additional experiments were performed to analyze the binding of the anti-DLL3/anti-CD3/anti-HSA trispecific binding construct to multiple cancer cell lines including NCI-H1299 parental, NCI-H1299 transduced with human DLL3 (NCI-H1299-hDLL3), SHP-77 and NCI-H82. Firstly, the cancer cell lines were stained for human DLL3 expression. For each cell line, 5×10⁴ cells/well were plated in a 96 well round plate. Cells were incubated for 15 min at 4° C. with 30 μL/well of anti-DLL3 antibody (Biolegend, clone RMD3-13, cat #154004) in Flow media (PBS, 1% BSA). Samples were washed with 0.25 mL Flow media, then 100 μL Flow media was added. 5 μL 7AAD was added 5 min before plate analyzing to enable live/dead gating. Data were acquired on a BD LSRFortessa using BD FACSDiva software, and analyzed using FlowJo software. The results are shown in FIG. 5A. The analyzed cell lines express DLL3 in about 70%-90% of the cell population, with about 5000-7000 mean fluorescence intensity (MFI) within the positive cells. Binding assay was performed following procedures similar to the CHO cells above, and the results are shown in FIG. 5B and FIG. 5C. The anti-DLL3/anti-CD3/anti-HSA trispecific binding construct binds to all DLL3 expressing cell lines.

Table 11 below summarizes the binding EC50 values of the trispecific binding construct to DLL3 in various cell lines used in this example.

TABLE 11
EC50 of Trispecific Binding Construct Binding to DLL3 in Various Cell
Lines

| DLL3 binding | EC50 [nM] |
|---|---|
| CHO-human DLL3 | 5.56 ± 0.22 |
| CHO-Cyno DLL3 | 5.51 ± 1.35 |
| NCI-H1299-human DLL3 | 10.47 ± 1.5 |
| NCI-H82 | 9.96 ± 8.9 |
| SHP-77 | 12.36 ± 7.12 |

Table 12 below summarize the binding EC50 values of the trispecific binding construct to CD3 in various cell lines used in this example.

TABLE 12
EC50 of Trispecific Binding Construct Binding to CD3 in Various Cell
Lines

| CD3 binding | EC50 [nM] |
|---|---|
| Human CD4 T Cell | 17.97 ± 1.58 |
| Human CD8 T Cell | 18.11 ± 0.86 |
| Cyno CD4 T Cell | 7.29 ± 1.57 |
| Cyno CD8 T Cell | 6.57 ± 0.94 |

### Example 5: Cytotoxicity of the Anti-DLL3/Anti-CD3/Anti-HSA Trispecific Binding Construct

Experiments were performed to analyze the in vitro cytotoxicity of the anti-DLL3/anti-CD3/anti-HSA trispecific binding construct at the presence of T cells.

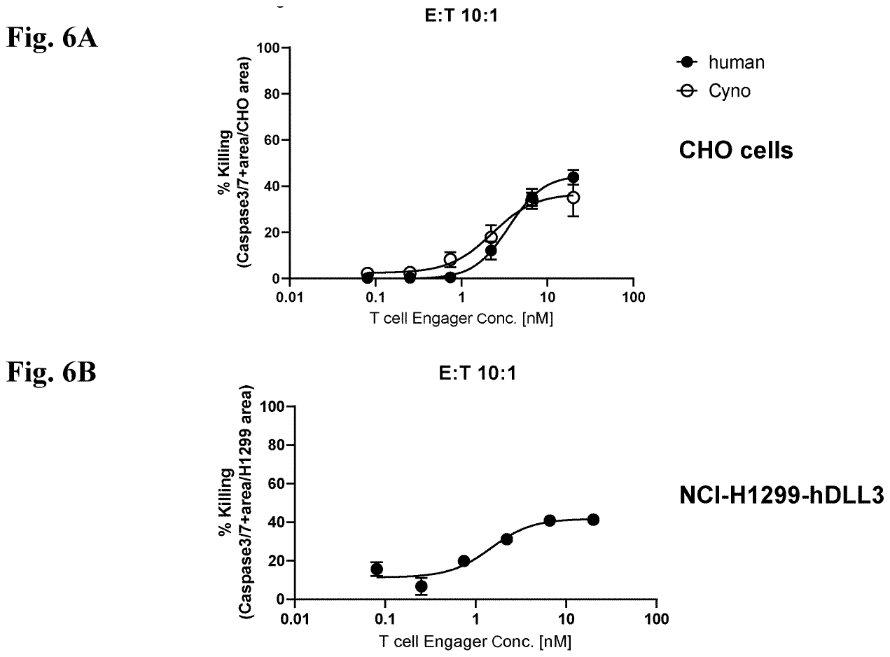

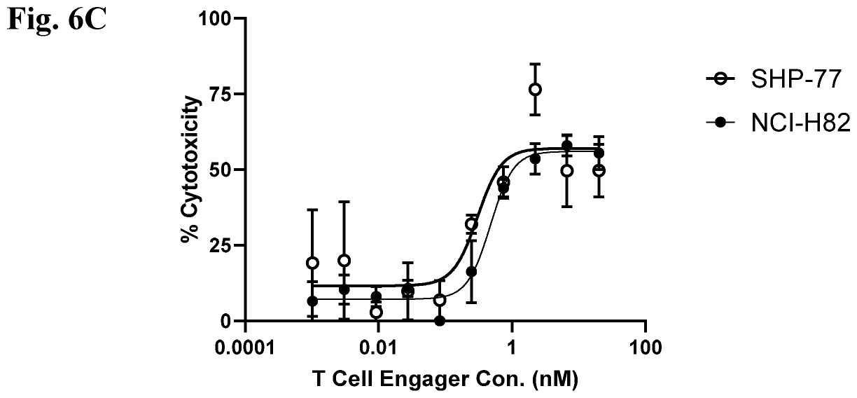

Protocol for CHO cells expressing human DLL3 or cyno, or NCI-H1299 cell line transduced with human DLL3. The day before the cytotoxicity assay, target cells were plated 5000 target cells/well in the 96 well plate. On the day of the assay, purified CD3 T cells (effector cell) from human or Cyno PBMCs were plated 50 000 cells/well, to obtain an Effector:Target cell ratio of 10:1. The trispecific binding construct was titrated from 20 nM to 0.1 nM, serial 3-fold dilutions in media and was added to the wells, then IncuCyte® Caspase-3/7 Green Reagent for Apoptosis (Essen BioScience, Inc.cat #4668) was added to the wells to measure cytotoxicity. The plates were scanned every 3 hours for 96 hours in the presence of IncuCyte®. The data was analyzed, and best time point was used to calculate the EC50 values. The results are shown in FIG. 6A and FIG. 6B.

Protocol for NCI-H82 and SHP-77 cell lines: NCI-H82 or SHP-77 target cells were plated at a density of 20000 and 3000 target cells/well, respectively, in flat bottom 96 well plate. Purified CD3 T cells from human PBMCs were plated to obtain an Effector:Target cell ratio of 5:1. The trispecific binding construct was titrated from 20 nM to 0.1 nM, serial 3-fold dilutions in media and was added to the wells. The cytotoxicity was measured 72 h later using the CytoTox 96® Non-Radioactive Cytotoxicity Assay (Promega, cat #G1780). The data was analyzed according to the manufacturer recommendations and EC50 values were calculated. The results are shown in FIG. 6C.

The results in FIG. 6A-6C demonstrate that the anti-DLL3/anti-CD3/anti-HSA trispecific binding construct is able to mediate potent cell killing activity of the DLL3 expressing target cells by T cells. The EC50 values of cytotoxicity evaluated in this example are summarized in Table 13 below.

TABLE 13
Cytotoxicity EC50 of Trispecific Binding Construct

| Target cells | EC50 [nM] |
|---|---|
| CHO-human DLL3 | 1.79 ± 0.9 |
| CHO-Cyno DLL3 | 1.58 ± 0.84 |
| NCI-H1299-human DLL3 | 1.49 ± 0.44 |
| NCI-H82 | 0.36 ± 0.19 |
| SHP-77 | 0.43 ± 0.28 |

### Example 6: Pharmacokinetics (PK) Study of the Anti-DLL3/Anti-CD3/Anti-HSA Trispecific Binding Construct

Experiments were performed to evaluate the pharmacokinetics of the trispecific binding construct and the effect of the anti-HSA binding domain.

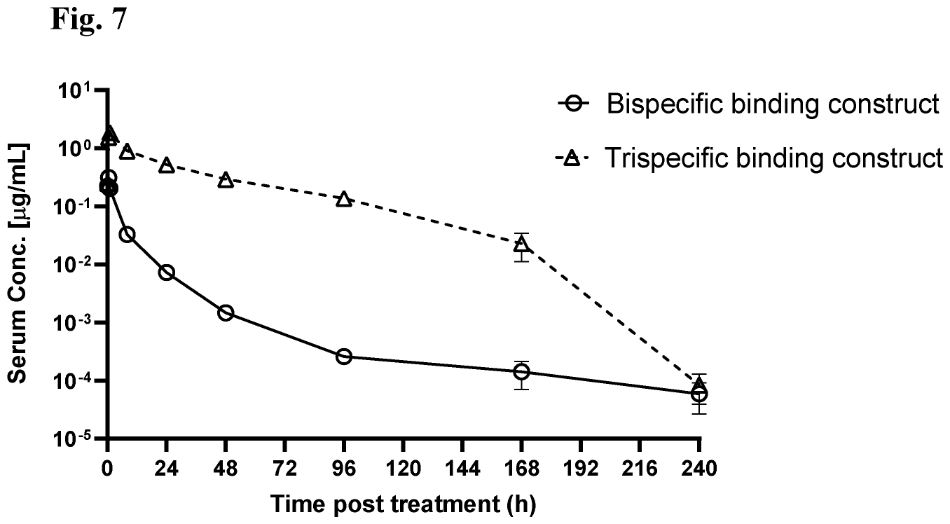

Balb/c mice were dosed with 1 mg/kg, single intravenous injection, 5 ml/kg volume, of either the anti-DLL3/anti-CD3/anti-HSA trispecific binding construct, or an anti-DLL3/anti-CD3 bispecific binding construct. Blood was collected at 5 min (Cmax), 1 hr, 8 hrs, 24 hrs, 48 hrs, 72 hrs, 96 hrs, 7 days, and 10 days post-injection. The serum was frozen before quantification. The concentrations of the binding constructs were measured using an in-house developed immunoassay on the MSD platform. Biotinylated capture antibody (anti-VHH, Genscript, #A02015) was linked to a streptavidin small spot plate (MSD, #R92TG). Detection anti-His-tag antibody (THE anti-HIS, Genscript, #A00186-100) was conjugated to a SULFO-TAG-NHS-Ester label (MSD, R31AA). Standard curve was generated using the same binding constructs used in the study at a concentration range from 10,000 μg/ml to 2.44 pg/ml. Mouse plasma samples were diluted with assay diluent (MSD Blocker A, #R93BA in PBS) as necessary for quantification within the range of detection, with a minimum dilution of 1:10. Samples were analyzed on the Quickplex SQ120. Standard curves and data analysis were generated using the MSD Discovery Workbench Analysis Software, v.4. The results are shown in FIG. 7 and Table 14 below.

TABLE 14
PK of Trispecific Binding Construct vs Bispecific Binding Construct

| | anti-DLL3/anti-CD3/anti- | anti-DLL3/anti-CD3 |
|---|---|---|
| | HSA trispecific binding | bispecific binding |
| | construct | construct |
| T1/2 (h) | 33.281 | 9.02 |
| AUC∞ (h * μg/mL) | 45.959 | 1.33 |
| Cl (mL/h/kg) | 21.76 | 751.32 |
| Vss (mL/kg) | 866.28 | 8259.21 |

T1/2: serum half-life;
AUC∞: cumulative area under curve;
Cl: clearance rate;
Vss: steady state volume of distribution.

The results demonstrate that the presence of anti-HSA binding domain significantly increases the serum retention time of the binding construct.

### Example 7: In Vivo Efficacy Studies of the Anti-DLL3/Anti-CD3/Anti-HSA Trispecific Binding Construct

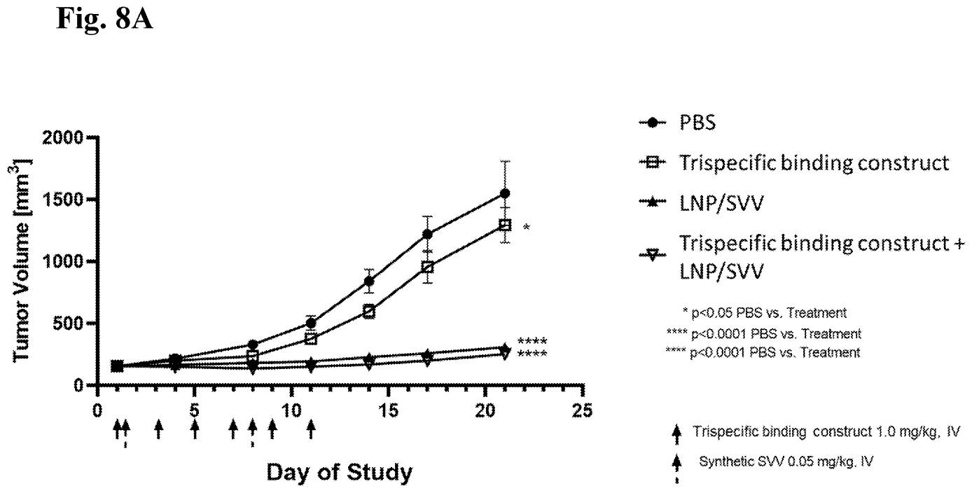

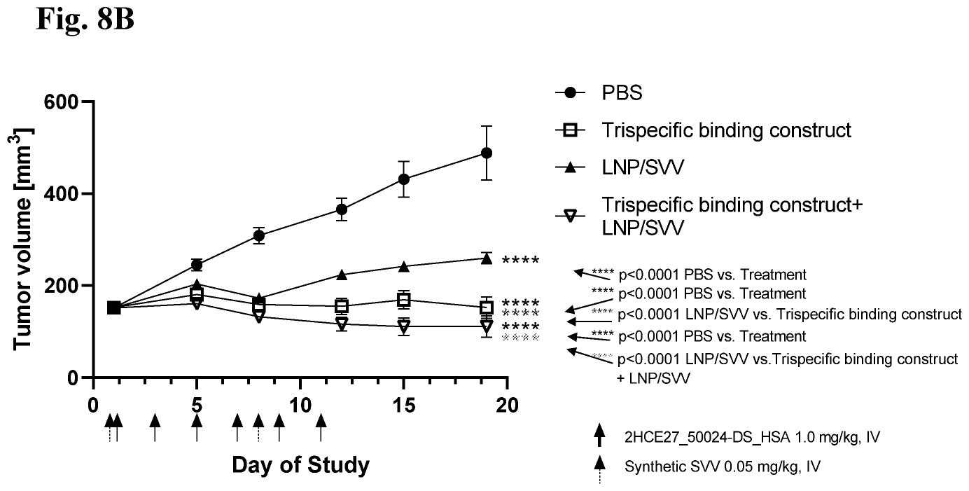

NSG mice (The Jackson Laboratory) 8 weeks old were injected with 5×10⁶-NCI-H82 or NCI-H1299hDLL3-tumor cells. 10×10⁶ freshly isolated PBMCs were intravenously injected 7 days prior treatment initiation. Treatments were initiated when mice reached an average tumor size of approximately 150 mm³. Mice were dosed with the anti-DLL3/anti-CD3/anti-HSA trispecific binding construct intravenously at 1.0 mg/kg every 2 days for 6 injections, and/or dosed twice with lipid nanoparticles (LNPs) containing synthetic SVV RNA viral genomes intravenously at 0.05 mg/kg every 7 days. Tumor volume and body weight were measured twice a week. Study endpoint was reached when the tumor volume reached 2000 mm³, or >20% body weight loss occurred, or tumors had open ulcerations, or mice reached 21 or 28 days from treatment initiation. The results are shown in FIG. 8A (for NCI-H82 tumor model) and FIG. 8B (for NCI-H1299-hDLL3 tumor model).

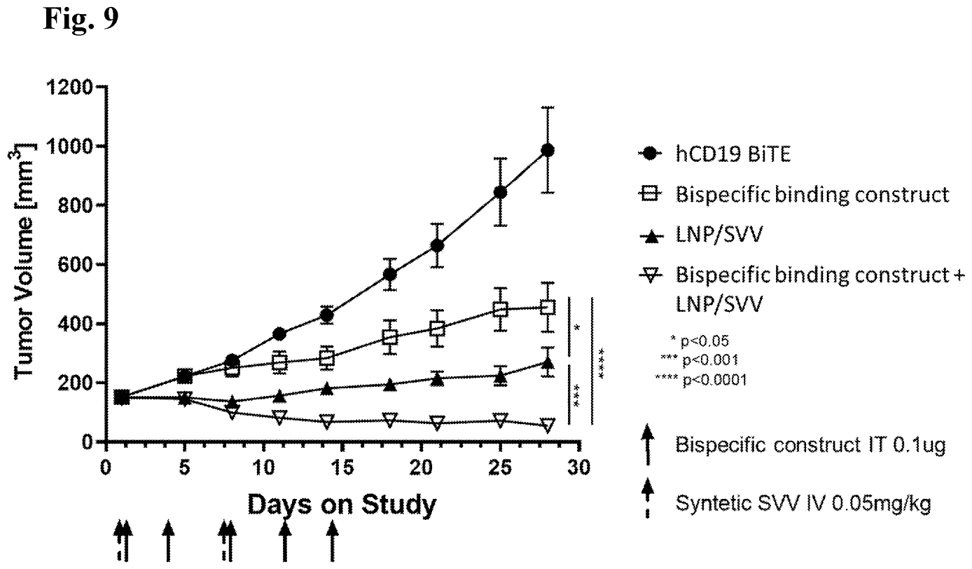

In another set of experiments, NSG mice (The Jackson Laboratory) 8 weeks old were injected with 5×10⁶ NCI-H1299-hDLL3 tumor cells. 10×10⁶ freshly isolated PBMCs were intravenously injected 7 days prior treatment initiation. Treatments were initiated when mice reached an average tumor size of approximately 150 mm³. Mice were dosed with an anti-DLL3/anti-CD3 bispecific binding construct intratumorally at 0.1 μg every 3 days for 5 injections, and/or dosed twice with lipid nanoparticles containing synthetic SVV RNA viral genome intravenously at 0.05 mg/kg every 7 days. A human CD19 BiTE was used as a control group. Tumor volume and body weight were measured twice a week. Study endpoint was reached when the tumor volume reached 2000 mm³, or >20% body weight loss occurred, or tumors had open ulcerations, or mice reached 21 or 28 days from treatment initiation. The results are shown in FIG. 9.

These results demonstrate that a T-cell engager molecule comprising a DLL3 binding domain and a CD3 binding domain can effectively mediate the killing of cancer cells in an NCI-H1299-hDLL3 lung cancer model, and its anti-cancer effect is synergistic with the treatment of an oncolytic virus such as SVV.

### Example 8: In Vivo Efficacy of Lipid Nanoparticles Comprising Oncolytic Virus-Encoding RNA Molecules in Combination with an Anti-DLL3 Antibody in Lung Cancer

The ability of anti-DLL3 antibodies (exemplary forms include full length monoclonal antibody, T-cell engager, T-cell engager with extended half-life, NK engager, or NK engager with extended half-life) to enhance the efficacy of a lipid nanoparticles comprising Coxsackie A virus (CVA)-encoding RNA molecules to inhibit lung tumor growth in vivo are evaluated.

Briefly, 8-week-old NSG mice are injected with human PBMC on day 1, 2 and 3. On day 10, NCI-H1299-hDLL3 cells (5×10⁶ cells/0.1 mL in a 1:1 mixture of serum-free PBS and Matrigel®) are implanted subcutaneously in the right flank of PBMC-humanized mice. When median tumor size is approximately 150 mm³ (120-180 mm³ range), mice are cohorted in groups of 8-10 mice per treatment arm. Mice are treated with the anti-DLL3 antibody (via intravenous or intratumoral administration) and/or intravenous administration of CVA-RNA lipid nanoparticles. Tumor volume is measured 2 times a week to assess the efficacy of each treatment arm.

### Example 9: In Vivo Efficacy of Lipid Nanoparticles Comprising Oncolytic Virus-Encoding RNA Molecules in Combination with Anti-DLL3 Antibody Encoded by an mRNA in Lung Cancer

mRNA encoding an anti-DLL3 antibody (exemplary forms include full-length monoclonal antibody, T-cell engager, T-cell engager with extended half-life, NK engager, or NK engager with extended half-life) and SVV-RNA or CVA-RNA are co-formulated in lipid nanoparticles to evaluate their efficacy to inhibit lung tumor growth in vivo.

Briefly, 8-week-old NSG mice are injected with human PBMC on day 1, 2 and 3. On day 10, NCI-H1299-hDLL3 cells (5×10⁶ cells/0.1 mL in a 1:1 mixture of serum-free PBS and Matrigel®) are implanted subcutaneously in the right flank of PBMC-humanized mice. When median tumor size is approximately 150 mm³ (120-180 mm³ range), mice are cohorted in groups of 8-10 mice per treatment arm. Mice are treated with intravenous administration of SVV-RNA or CVA-RNA lipid nanoparticles or SVV-RNA or CVA-RNA lipid nanoparticles co-formulated with mRNA encoding the anti-DLL3 antibody. Tumor volume is measured 2 times a week to assess the efficacy of each treatment arm.

### Example 10: In Vivo Efficacy of Lipid Nanoparticles Comprising Oncolytic Virus-Encoding RNA Molecules in Lung Cancer

An oncolytic virus-RNA genome (e.g., SVV-RNA or CVA-RNA) comprising a transgene encoding an anti-DLL3 antibodies (for example, in the form of T-cell engager, or NK engager) is formulated in a lipid nanoparticle. Alternatively, a replicon comprising a transgene encoding an anti-DLL3 antibody is co-formulated with oncolytic virus-encoding RNA (e.g., SVV-RNA or CVA-RNA) in a lipid nanoparticle. These lipid nanoparticles are evaluated for their efficacy to inhibit lung tumor growth in vivo.

Briefly, 8-week-old NSG mice are injected with human PBMC on day 1, 2 and 3. On day 10, NCI-H1299-hDLL3 cells (5×10⁶ cells/0.1 mL in a 1:1 mixture of serum-free PBS and Matrigel®) are implanted subcutaneously in the right flank of PBMC-humanized mice. When median tumor size reached approximately 150 mm³ (120-180 mm³ range), mice are cohorted in groups of 8-10 mice per treatment arm. Mice are treated with intravenous administration of lipid nanoparticles containing oncolytic virus-encoding RNA (without transgene encoding the anti-DLL3 antibody), lipid nanoparticles containing oncolytic virus-encoding RNA comprising a transgene encoding the anti-DLL3 antibody, or lipid nanoparticles co-formulated with oncolytic virus-encoding RNA and replicons comprising a transgene encoding the anti-DLL3 antibody. Tumor volume is measured 2 times a week to assess the efficacy of each treatment arm.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A nucleic acid encoding a protein binding construct comprising a DLL3-binding domain that is capable of specifically binding DLL3 or an epitope of DLL3 comprising a heavy chain variable region (VH) comprising a complementarity determining region (CDR) 1, a CDR2, and a CDR3, wherein the CDR1, CDR2, and CDR3 comprise the amino acid sequences as set forth in any one of the following sets of SEQ ID NOs:
a) SEQ ID NOs: 46, 47, and 48, respectively;
b) SEQ ID NOs: 52, 53, and 54, respectively;
c) SEQ ID NOs: 18, 19, and 20, respectively;
d) SEQ ID NOs: 83, 2, and 84, respectively;
e) SEQ ID NOs: 60, 61, and 62, respectively;
f) SEQ ID NOs: 65, 66, and 67, respectively;
g) SEQ ID NOs: 69, 23, and 24, respectively;
h) SEQ ID NOs: 14, 43, and 71, respectively;
i) SEQ ID NOs: 73, 74, and 75, respectively;
j) SEQ ID NOs: 77, 78, and 79, respectively;
k) SEQ ID NOs: 1, 2, and 3, respectively;
l) SEQ ID NOs: 6, 7, and 8, respectively;
m) SEQ ID NOs: 10, 11, and 12, respectively;
n) SEQ ID NOs: 14, 15, and 16, respectively;
o) SEQ ID NOs: 22, 23, and 24, respectively;
p) SEQ ID NOs: 26, 27, and 28, respectively;
q) SEQ ID NOs: 30, 2, and 31, respectively;
r) SEQ ID NOs: 33, 81, and 34, respectively;
s) SEQ ID NOs: 36, 82, and 37, respectively;
t) SEQ ID NOs: 39, 40, and 41, respectively; and
u) SEQ ID NOs: 14, 43, and 44, respectively.

2. The nucleic acid of claim 1, wherein the protein binding construct further comprises a human framework region sequence.

3. The nucleic acid of claim 1, wherein the protein binding construct comprises an amino acid sequence that is at least 95% identical to any one of SEQ ID NOs: 4, 5, 9, 13, 17, 21, 25, 29, 32, 35, 38, 42, 45, 49, 55, 59, 63, 64, 68, 70, 72, and 76.

4. The nucleic acid of claim 1, wherein the protein binding construct comprises an amino acid sequence that is at least 99% identical to any one of SEQ ID NOs: 4, 5, 9, 13, 17, 21, 25, 29, 32, 35, 38, 42, 45, 49, 55, 59, 63, 64, 68, 70, 72, and 76.

5. The nucleic acid of claim 1, wherein the protein binding construct is a chimeric antigen receptor or a recombinant T-cell receptor.

6. The nucleic acid of claim 1, wherein the protein binding construct further comprises an additional binding domain.

7. The nucleic acid of claim 6, wherein the protein binding construct is bispecific.

8. The nucleic acid of claim 1, wherein the protein binding construct further comprises two additional binding domains.

9. The nucleic acid of claim 8, wherein the protein binding construct is trispecific.

10. A composition comprising the nucleic acid of claim 1.

11. A vector comprising the nucleic acid of claim 1.

12. The vector of claim 11, wherein the vector is a viral vector.

13. A cell comprising the nucleic acid of claim 1.

14. A cell comprising the nucleic acid of claim 5.

15. A bispecific protein binding construct comprising a first binding domain and a second binding domain, wherein the first binding domain is a DLL3-binding domain that is capable of specifically binding DLL3 or an epitope of DLL3 and comprises a VH comprising a CDR1, a CDR2, and a CDR3, wherein the CDR1, CDR2, and CDR3 comprise the amino acid sequences as set forth in any one of the following sets of SEQ ID NOs:
a) SEQ ID NOs: 46, 47, and 48, respectively;
b) SEQ ID NOs: 52, 53, and 54, respectively;
c) SEQ ID NOs: 18, 19, and 20, respectively;
d) SEQ ID NOs: 83, 2, and 84, respectively;
e) SEQ ID NOs: 60, 61, and 62, respectively;
f) SEQ ID NOs: 65, 66, and 67, respectively;
g) SEQ ID NOs: 69, 23, and 24, respectively;
h) SEQ ID NOs: 14, 43, and 71, respectively;
i) SEQ ID NOs: 73, 74, and 75, respectively;
j) SEQ ID NOs: 77, 78, and 79, respectively;
k) SEQ ID NOs: 1, 2, and 3, respectively;
l) SEQ ID NOs: 6, 7, and 8, respectively;
n) SEQ ID NOs: 10, 11, and 12, respectively;
n) SEQ ID NOs: 14, 15, and 16, respectively;
o) SEQ ID NOs: 22, 23, and 24, respectively;
p) SEQ ID NOs: 26, 27, and 28, respectively;
q) SEQ ID NOs: 30, 2, and 31, respectively;
r) SEQ ID NOs: 33, 81, and 34, respectively;
s) SEQ ID NOs: 36, 82, and 37, respectively;
t) SEQ ID NOs: 39, 40, and 41, respectively; and
u) SEQ ID NOs: 14, 43, and 44, respectively.

16. The bispecific binding construct of claim 15, wherein the second binding domain is an antigen binding domain that specifically binds to CD3, CD16, NKp46, human serum albumin, or an Fc domain.

17. A trispecific binding construct comprising a first binding domain, a second binding domain, and a third binding domain, wherein the wherein the first binding domain is a DLL3-binding domain that is capable of specifically binding DLL3 or an epitope of DLL3 and comprises a VH comprising a CDR1, a CDR2, and a CDR3, wherein the CDR1, CDR2, and CDR3 comprise the amino acid sequences as set forth in any one of the following sets of SEQ ID NOs:
a) SEQ ID NOs: 46, 47, and 48, respectively;
b) SEQ ID NOs: 52, 53, and 54, respectively;
c) SEQ ID NOs: 18, 19, and 20, respectively;
d) SEQ ID NOs: 83, 2, and 84, respectively;
e) SEQ ID NOs: 60, 61, and 62, respectively;
f) SEQ ID NOs: 65, 66, and 67, respectively;
g) SEQ ID NOs: 69, 23, and 24, respectively;
h) SEQ ID NOs: 14, 43, and 71, respectively;
i) SEQ ID NOs: 73, 74, and 75, respectively;
j) SEQ ID NOs: 77, 78, and 79, respectively;
k) SEQ ID NOs: 1, 2, and 3, respectively;
l) SEQ ID NOs: 6, 7, and 8, respectively;
o) SEQ ID NOs: 10, 11, and 12, respectively;
n) SEQ ID NOs: 14, 15, and 16, respectively;
o) SEQ ID NOs: 22, 23, and 24, respectively;
p) SEQ ID NOs: 26, 27, and 28, respectively;
q) SEQ ID NOs: 30, 2, and 31, respectively;
r) SEQ ID NOs: 33, 81, and 34, respectively;
s) SEQ ID NOs: 36, 82, and 37, respectively;
t) SEQ ID NOs: 39, 40, and 41, respectively; and
u) SEQ ID NOs: 14, 43, and 44, respectively.

18. The trispecific binding construct of claim 17, wherein:
the second binding domain is an antigen binding domain that specifically binds to human serum albumin; and
the third binding domain is an antigen binding domain that specifically binds to CD3, CD16, or NKp46.

19. A method of treating cancer in a subject in need thereof comprising administering an effective amount of the cell of claim 13 to the subject.

20. A method of treating cancer in a subject in need thereof comprising administering an effective amount of the cell of claim 14 to the subject.


SEQUENCE LISTING (761 sequences)

SEQ ID NO: 1 - protein, 8 aa, synthetic construct
  REGION 1..8: note = VH-CDR1
GSIVGDYA

SEQ ID NO: 2 - protein, 7 aa, synthetic construct
  REGION 1..7: note = VH-CDR2
IGSEGSR

SEQ ID NO: 3 - protein, 9 aa, synthetic construct
  REGION 1..9: note = VH-CDR3
FLYNSGEDY

SEQ ID NO: 4 - protein, 115 aa, synthetic construct
  REGION 1..115: note = 2DLT10
QVQLQESGGGLVQPGESLRLSCAASGSIVGDYAMGWYRQAPGKKRELVAGIGSEGSRDYADSVKGRFTISRDNAKRTLYLQMNSLKSEDTAVYICFLYNSGEDYWGQGTQVTVSS

SEQ ID NO: 5 - protein, 115 aa, synthetic construct
  REGION 1..115: note = 1DLT176
QVQLQESGGGLVQPGGSLRLACAASGSIVGDYAMGWYRQAPGKKRELVAGIGSEGSRDYADSVKGRFTISRDNAKRTLYLQMNSLKSEDTAVYICFLYNSGEDYWGQGTQVTVSS

SEQ ID NO: 6 - protein, 8 aa, synthetic construct
  REGION 1..8: note = VH-CDR1
GFTTDDYG

SEQ ID NO: 7 - protein, 7 aa, synthetic construct
  REGION 1..7: note = VH-CDR2
ITTGGST

SEQ ID NO: 8 - protein, 19 aa, synthetic construct
  REGION 1..19: note = VH-CDR3
NAVCSGSGCYEVSWESYDY

SEQ ID NO: 9 - protein, 125 aa, synthetic construct
  REGION 1..125: note = 2HCE138
QVQLQESGGGLVQAGGSLRLSCAVSGFTTDDYGIGWFRQAPGKQRELVAIITTGGSTNYADSVKGRFKISRDNAKNTVYLQMNSLKPEDTAVYYCNAVCSGSGCYEVSWESYDYWGQGTQVTVSS

SEQ ID NO: 10 - protein, 8 aa, synthetic construct
  REGION 1..8: note = VH-CDR1
GRTYSNYF

SEQ ID NO: 11 - protein, 8 aa, synthetic construct
  REGION 1..8: note = VH-CDR2
VSWSGDRT

SEQ ID NO: 12 - protein, 11 aa, synthetic construct
  REGION 1..11: note = VH-CDR3
AAGPLINRINY

SEQ ID NO: 13 - protein, 118 aa, synthetic construct
  REGION 1..118: note = 3HCE7
QVQLQESGGGLVQAGDSLRLSCAGSGRTYSNYFIDWFRQAPGKEREFVAAVSWSGDRTTYADSVKGRFTVSRDNAKNTWYLQMNSLKPEDTAVYYCAAGPLINRINYWGQGTQVTVSS

SEQ ID NO: 14 - protein, 8 aa, synthetic construct
  REGION 1..8: note = VH-CDR1
GFTFSSYA

SEQ ID NO: 15 - protein, 8 aa, synthetic construct
  REGION 1..8: note = VH-CDR2
IDSGGGST

SEQ ID NO: 16 - protein, 10 aa, synthetic construct
  REGION 1..10: note = VH-CDR3
AKEPWVVADY

SEQ ID NO: 17 - protein, 117 aa, synthetic construct
  REGION 1..117: note = 2HCE151
QVQLQESGGGLVQPGGSLRLSCVASGFTFSSYAMSWVRQAPGKGPEWVSRIDSGGGSTSYADSVKGRFTISRDNAKNTLHLQMNGLKPEDTAVYYCAKEPWVVADYWGQGTQVTVSS

SEQ ID NO: 18 - protein, 8 aa, synthetic construct
  REGION 1..8: note = VH-CDR1
GRAGSSYD

SEQ ID NO: 19 - protein, 7 aa, synthetic construct
  REGION 1..7: note = VH-CDR2
ITWSGNT

SEQ ID NO: 20 - protein, 11 aa, synthetic construct
  REGION 1..11: note = VH-CDR3
AAALSEKKYEY

SEQ ID NO: 21 - protein, 117 aa, synthetic construct
  REGION 1..117: note = 3HCE4
QVQLQESGGGLVQAGASLKLSCAVSGRAGSSYDMGWLRQAPGKEREFVAIITWSGNTAYKDSVKGRFIISRDNAKNTVYLEMNSLAPEDTAVYYCAAALSEKKYEYWGQGTQVTVSS

SEQ ID NO: 22 - protein, 8 aa, synthetic construct
  REGION 1..8: note = VH-CDR1
GSISSIIS

SEQ ID NO: 23 - protein, 8 aa, synthetic construct
  REGION 1..8: note = VH-CDR2
AITSGGST

SEQ ID NO: 24 - protein, 13 aa, synthetic construct
  REGION 1..13: note = VH-CDR3
NAHVRDYSGSAYY

SEQ ID NO: 25 - protein, 120 aa, synthetic construct
  REGION 1..120: note = 3HCE80
QVQLQESGGGLVQPGGSLRLSCAASGSISSIISMGWYRQAPGKQRGELVAAITSGGSTSYAGSVEGRFAISRDSAKNTAYLQMNSLKPEDTAVYYCNAHVRDYSGSAYYTGQGTQVTVSS

SEQ ID NO: 26 - protein, 8 aa, synthetic construct
  REGION 1..8: note = VH-CDR1
GGTFSNYD

SEQ ID NO: 27 - protein, 8 aa, synthetic construct
  REGION 1..8: note = VH-CDR2
VNRYGDYS

SEQ ID NO: 28 - protein, 12 aa, synthetic construct
  REGION 1..12: note = VH-CDR3
AARLWNSAKYAY

SEQ ID NO: 29 - protein, 119 aa, synthetic construct
  REGION 1..119: note = 3HCE86
QVQLQESGGGLVQPGGSLRLSCAASGGTFSNYDIAWFRQAPGKEREFVAAVNRYGDYSYYADSVKGRFTISRDNAKSTVSLQMNSLKPEDTFSYYCAARLWNSAKYAYWGQGTQVTVSS

SEQ ID NO: 30 - protein, 8 aa, synthetic construct
  REGION 1..8: note = VH-CDR1
GSIFSIYA

SEQ ID NO: 31 - protein, 15 aa, synthetic construct
  REGION 1..15: note = VH-CDR3
YAGRPPSASYSGAHY

SEQ ID NO: 32 - protein, 121 aa, synthetic construct
  REGION 1..121: note = 3DLT81
QVQLQESGGGLVQPGGSLRLSCAASGSIFSIYAMGWYRQAPGKKRELVAGIGSEGSRDYADSVKGRFTISRDNAKRTVYLQMNTLQPEDTAVYYCYAGRPPSASYSGAHYWGQGTQVTVSS

SEQ ID NO: 33 - protein, 8 aa, synthetic construct
  REGION 1..8: note = VH-CDR1
GFTFDDVA

SEQ ID NO: 34 - protein, 12 aa, synthetic construct
  REGION 1..12: note = VH-CDR3
AARRDSRGQYHD

SEQ ID NO: 35 - protein, 118 aa, synthetic construct
  REGION 1..118: note = 3HCE38
QVQLQESGGGLVQPGGSLRLSCAASGFTFDDVAMSWVRQAPGKQRELVAVITSGGSTYTADSVKGRFAISRDNAKNTVYLQMNSLKPEDTALYYCAARRDSRGQYHDWGQGTQVTVSS

SEQ ID NO: 36 - protein, 8 aa, synthetic construct
  REGION 1..8: note = VH-CDR1
GFTFSNSP

SEQ ID NO: 37 - protein, 12 aa, synthetic construct
  REGION 1..12: note = VH-CDR3
AKGATPSVLYDY

SEQ ID NO: 38 - protein, 119 aa, synthetic construct
  REGION 1..119: note = 2HCE6
QVQLQESGGGLVQTGGSLRLSCAASGFTFSNSPMSWVRRAPGKGPEWVSAISSNGRNTSYADSVKGRFTISRDNAKNTLFLQMHSLKFEDTAVYYCAKGATPSVLYDYGGQGTQVTVSS

SEQ ID NO: 39 - protein, 8 aa, synthetic construct
  REGION 1..8: note = VH-CDR1
GRIFSMFP

SEQ ID NO: 40 - protein, 7 aa, synthetic construct
  REGION 1..7: note = VH-CDR2
ITTGGRP

SEQ ID NO: 41 - protein, 14 aa, synthetic construct
  REGION 1..14: note = VH-CDR3
NAVSTPELAYPYDY

SEQ ID NO: 42 - protein, 120 aa, synthetic construct
  REGION 1..120: note = 1DLT39
QVQLQESGGGLVQPGGSLRLSCAASGRIFSMFPMGWYRQAPGKQRELVADITTGGRPNYADSVKGRFTITRDNAKNTVYLQMNSLQPEDTAVYFCNAVSTPELAYPYDYWGQGTQVTVSS

SEQ ID NO: 43 - protein, 8 aa, synthetic construct
  REGION 1..8: note = VH-CDR2
INSGGGST

SEQ ID NO: 44 - protein, 10 aa, synthetic construct
  REGION 1..10: note = VH-CDR3
AKEPWVTQGS

SEQ ID NO: 45 - protein, 117 aa, synthetic construct
  REGION 1..117: note = 3HCE25
QVQLQESGGGLVQPGGSLRLSCEASGFTFSSYAMSWVRQAPGKGPEWVSRINSGGGSTSYADSVKGRFTISRDNAKNTLYLQMNSLKPEDTAVYYCAKEPWVTQGSWGQGTQVTVSS

SEQ ID NO: 46 - protein, 8 aa, synthetic construct
  REGION 1..8: note = VH-CDR1
GRTFRSYA

SEQ ID NO: 47 - protein, 8 aa, synthetic construct
  REGION 1..8: note = VH-CDR2
IIMSDGST

SEQ ID NO: 48 - protein, 13 aa, synthetic construct
  REGION 1..13: note = VH-CDR3
AARRDYFTGVYDY

SEQ ID NO: 49 - protein, 120 aa, synthetic construct
  REGION 1..120: note = 2HCE27
QVQLQESGGGLVQAGGSLRLSCAASGRTFRSYAMGWFRQAPGKEREFIAVIIMSDGSTSYADSVKGRFTISRDNAKNTLYLQMNSLKPEDTAVYYCAARRDYFTGVYDYWGQGTQVTVSS

SEQ ID NO: 50 - protein, 120 aa, synthetic construct
  REGION 1..120: note = 2HCE27 14.1
EVQLVESGGGLVQPGGSLRLSCAASGRTFRSYAMGWFRQAPGKEREFIAVIIMSDGSTSYADSVKGRFTISRDNAKNTLYLQMNSLKPEDTAVYYCAARRDYFTGVYDYWGQGTLVTVSS

SEQ ID NO: 51 - protein, 120 aa, synthetic construct
  REGION 1..120: note = 2HCE27 14.2
EVQLVESGGGLVQPGGSLRLSCAASGRTFRSYAMGWFRQAPGKEREFIAVIIMSDGSTSYADSVKGRFTISRDNAKNTLYLQMNSLRPEDTAVYYCAARRDYFTGVYDYWGQGTLVTVSS

SEQ ID NO: 52 - protein, 8 aa, synthetic construct
  REGION 1..8: note = VH-CDR1
GRTFSVDA

SEQ ID NO: 53 - protein, 8 aa, synthetic construct
  REGION 1..8: note = VH-CDR2
IDWTGGST

SEQ ID NO: 54 - protein, 12 aa, synthetic construct
  REGION 1..12: note = VH-CDR3
AARERSRTAYDY

SEQ ID NO: 55 - protein, 119 aa, synthetic construct
  REGION 1..119: note = 3HCE56
QVQLQESGGGLVQAGGSLRLSCAASGRTFSVDAMGWLRQAPGKEREFVVVIDWTGGSTAYADSVKGRFTISRDNARNTVYLQMNNLKPEDTGVYYCAARERSRTAYDYWGQGTQVTVSS

SEQ ID NO: 56 - protein, 119 aa, synthetic construct
  REGION 1..119: note = 3HCE56 15.1
EVQLVESGGGLVQPGGSLRLSCAASGRTFSVDAMGWLRQAPGKEREFVVVIDWTGGSTAYADSVKGRFTISRDNARNTVYLQMNNLKPEDTGVYYCAARERSRTAYDYWGQGTLVTVSS

SEQ ID NO: 57 - protein, 119 aa, synthetic construct
  REGION 1..119: note = 3HCE56 15.2
EVQLVESGGGLVQPGGSLRLSCAASGRTFSVDAMGWLRQAPGKEREFVVVIDWTGGSTAYADSVKGRFTISRDNARNTVYLQMNSLRPEDTAVYYCAARERSRTAYDYWGQGTLVTVSS

SEQ ID NO: 58 - protein, 119 aa, synthetic construct
  REGION 1..119: note = 3HCE56 15.3
EVQLVESGGGLVQPGGSLRLSCAASGRTFSVDAMGWLRQAPGKEREFVVVIDWTGGSTAYADSVKGRFTISRDNAKNTVYLQMNSLRPEDTAVYYCAARERSRTAYDYWGQGTLVTVSS

SEQ ID NO: 59 - protein, 115 aa, synthetic construct
  REGION 1..115: note = 2DLT2
QVQLQESGGGLVQPGGSLRLSCAASGSIVGNYAMGWYRQAPGKERELVAGIGSEGSRDYADSVKGRFTISRDTAKRTVYLQMNSLKSEDTAVYICFLYNSGDDYWGQGTQVTVSS

SEQ ID NO: 60 - protein, 8 aa, synthetic construct
  REGION 1..8: note = VH-CDR1
GSIFSINS

SEQ ID NO: 61 - protein, 7 aa, synthetic construct
  REGION 1..7: note = VH-CDR2
KASDGST

SEQ ID NO: 62 - protein, 9 aa, synthetic construct
  REGION 1..9: note = VH-CDR3
FLYANNIPY

SEQ ID NO: 63 - protein, 115 aa, synthetic construct
  REGION 1..115: note = 3HCE44
QVQLQESGGGSVQAGGSLRLSCAASGSIFSINSMGWHRHAPGKQRELVAAKASDGSTNYAGPVRGRFTITSDDAKNTVYLQMNNLKPEDTAVYYCFLYANNIPYWAKGTQVTVSS

SEQ ID NO: 64 - protein, 115 aa, synthetic construct
  REGION 1..115: note = 2HCE174
QVQLQESGGGSVQAGGSLRLSCAASGSIFSINSMGWHRHAPGKQRELVAAKASDGSTNYAGPVRGRFTITSDDAKNTVYLQMNSLKPEDTAVYYCFLYANNIPYWAKGTQVTVSS

SEQ ID NO: 65 - protein, 8 aa, synthetic construct
  REGION 1..8: note = VH-CDR1
GRTFSSYA

SEQ ID NO: 66 - protein, 8 aa, synthetic construct
  REGION 1..8: note = VH-CDR2
ISGSGYSA

SEQ ID NO: 67 - protein, 14 aa, synthetic construct
  REGION 1..14: note = VH-CDR3
AARNERGASSSYDY

SEQ ID NO: 68 - protein, 121 aa, synthetic construct
  REGION 1..121: note = 2HCE167
QVQLQESGGGLVQAGGSLRLSCAASGRTFSSYAMGWLRQAPGKEREFVAVISGSGYSASYRDSVKGRFTISRDNSKNTVFLQMNSLKPEDTAVYYCAARNERGASSSYDYWGQGTQVTVSS

SEQ ID NO: 69 - protein, 8 aa, synthetic construct
  REGION 1..8: note = VH-CDR1
GSIFSIIT

SEQ ID NO: 70 - protein, 120 aa, synthetic construct
  REGION 1..120: note = 3HCE18
QVQLQESGGGLVQAGGSLRLSCAASGSIFSIITMGWYRQAPGKQRGELVAAITSGGSTSYADSVKGRFAISRDSAKNTAYLQMNSLKPEDTAVYYCNAHVRDYSGSAYYTGQGTQVTVSS

SEQ ID NO: 71 - protein, 8 aa, synthetic construct
  REGION 1..8: note = VH-CDR3
ATPFEIGS

SEQ ID NO: 72 - protein, 115 aa, synthetic construct
  REGION 1..115: note = 2HCE117
QVQLQESGGGLVQPGGSLRLSCAASGFTFSSYAMSWVRQAPGKGLEWVSDINSGGGSTYYADSVKGRFTISRDNAKNTLYLQMDSLKPEDTAVYYCATPFEIGSWGQGTQVTVSS

SEQ ID NO: 73 - protein, 8 aa, synthetic construct
  REGION 1..8: note = VH-CDR1
GFIFDDYI

SEQ ID NO: 74 - protein, 8 aa, synthetic construct
  REGION 1..8: note = VH-CDR2
ISWSGSAT

SEQ ID NO: 75 - protein, 16 aa, synthetic construct
  REGION 1..16: note = VH-CDR3
AASSRGPYNSGSSYDY

SEQ ID NO: 76 - protein, 123 aa, synthetic construct
  REGION 1..123: note = 3HCE87
QVQLQESGGGLVQAGGSLRLSCAASGFIFDDYIIGWFRQAPGKEREFVAAISWSGSATAYADSVKGRFTISRDNAKNTLYLQMNTLKPEDTAVYYCAASSRGPYNSGSSYDYWGQGTQVTVSS

SEQ ID NO: 77 - protein, 8 aa, synthetic construct
  REGION 1..8: note = VH-CDR1
GYTFTDYA

SEQ ID NO: 78 - protein, 8 aa, synthetic construct
  REGION 1..8: note = VH-CDR2
INTYTGKP

SEQ ID NO: 79 - protein, 13 aa, synthetic construct
  REGION 1..13: note = VH-CDR3
SRERGYYDYSRSD

SEQ ID NO: 80 - protein, 120 aa, synthetic construct
  REGION 1..120: note = Anti-DLL3-1
QIQLVQSGPELKKPGESVKISCKASGYTFTDYAMHWVKQAPGKGLKWMGWINTYTGKPTYADDFKGRFVLSLEASASTSTLQISDLRNEDTAIYFCSRERGYYDYSRSDWGQGTLVTVSS

SEQ ID NO: 81 - protein, 7 aa, synthetic construct
  REGION 1..7: note = VH-CDR2
ITSGGST

SEQ ID NO: 82 - protein, 8 aa, synthetic construct
  REGION 1..8: note = VH-CDR2
ISSNGRNT

SEQ ID NO: 83 - protein, 8 aa, synthetic construct
  REGION 1..8: note = VH-CDR1
GSIVGNYA

SEQ ID NO: 84 - protein, 9 aa, synthetic construct
  REGION 1..9: note = VH-CDR3
FLYNSGDDY

SEQ ID NO: 85 - protein, 20 aa, synthetic construct
  REGION 1..20: note = STD1
NYGGGGSGGGGSGGGGSGNS

SEQ ID NO: 86 - protein, 38 aa, synthetic construct
  REGION 1..38: note = STD2
NYGGGGSGGGGSGGGGSGNYGGGGSGGGGSGGGGSGNS

SEQ ID NO: 87 - skipped

SEQ ID NO: 88 - protein, 8 aa, synthetic construct
  REGION 1..8: note = H2
GGGGSGNS

SEQ ID NO: 89 - protein, 10 aa, synthetic construct
  REGION 1..10: note = H3
NYGGGGSGNS

SEQ ID NO: 90 - protein, 13 aa, synthetic construct
  REGION 1..13: note = H4
GGGGSGGGGSGNS

SEQ ID NO: 91 - protein, 15 aa, synthetic construct
  REGION 1..15: note = H5
NYGGGGSGGGGSGNS

SEQ ID NO: 92 - protein, 18 aa, synthetic construct
  REGION 1..18: note = H6
GGGGSGGGGSGGGGSGNS

SEQ ID NO: 93 - protein, 8 aa, synthetic construct
  REGION 1..8: note = H7
GCPPCPNS

SEQ ID NO: 94 - protein, 5 aa, synthetic construct
  REGION 1..5: note = Gly4Ser
GGGGS

SEQ ID NO: 95 - protein, 15 aa, synthetic construct
  REGION 1..15: note = (G4S)3
GGGGSGGGGSGGGGS

SEQ ID NO: 96 - protein, 16 aa, synthetic construct
  REGION 1..16: note = H105
SGGGGSGGGGSGGGGS

SEQ ID NO: 97 - protein, 20 aa, synthetic construct
  REGION 1..20: note = (G4S)4
GGGGSGGGGSGGGGSGGGGS

SEQ ID NO: 98 - protein, 21 aa, synthetic construct
  REGION 1..21: note = H75 (NKG2A quadruple mutant)
QRHNNSSLNTGTQMAGHSPNS

SEQ ID NO: 99 - protein, 16 aa, synthetic construct
  REGION 1..16: note = H83 (NKG2A derived)
SSLNTGTQMAGHSPNS

SEQ ID NO: 100 - protein, 18 aa, synthetic construct
  REGION 1..18: note = H106 (NKG2A derived)
QRHNNSSLNTGTQMAGHS

SEQ ID NO: 101 - protein, 14 aa, synthetic construct
  REGION 1..14: note = H81 (NKG2D derived)
EVQIPLTESYSPNS

SEQ ID NO: 102 - protein, 20 aa, synthetic construct
  REGION 1..20: note = H91 (NKG2D derived)
NSLANQEVQIPLTESYSPNS

SEQ ID NO: 103 - protein, 19 aa, synthetic construct
  REGION 1..19: note = H94
SGGGGSGGGGSGGGGSPNS

SEQ ID NO: 104 - protein, 19 aa, synthetic construct
  REGION 1..19: note = H111
SGGGGSGGGGSGGGGSPGS

SEQ ID NO: 105 - protein, 19 aa, synthetic construct
  REGION 1..19: note = H113
SGGGGSGGGGSGGGGSPAS

SEQ ID NO: 106 - protein, 18 aa, synthetic construct
  REGION 1..18: note = H114
SGGGGSGGGGSGGGGSPS

SEQ ID NO: 107 - protein, 19 aa, synthetic construct
  REGION 1..19: note = H115
SGGGGSGGGGSGGGGSPSS

SEQ ID NO: 108 - protein, 16 aa, synthetic construct
  REGION 1..16: note = H116
SGSETPGTSESATPES

SEQ ID NO: 109 - protein, 15 aa, synthetic construct
  REGION 1..15: note = H117
GGGGSGGGGSGGGGS

SEQ ID NO: 110 - protein, 15 aa, synthetic construct
  REGION 1..15: note = H118
AHHSEDPSSKAPKAP

SEQ ID NO: 111 - protein, 16 aa, synthetic construct
  REGION 1..16: note = H119
SPSTPPTPSPSTPPAS

SEQ ID NO: 112 - protein, 101 aa, synthetic construct
  REGION 1..101: note = CH3
EPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 113 - protein, 5 aa, synthetic construct
  REGION 1..5: note = HSA-1-CDR1
SFGMS

SEQ ID NO: 114 - protein, 17 aa, synthetic construct
  REGION 1..17: note = HSA-1-CDR2
SISGSGSDTLYADSVKG

SEQ ID NO: 115 - protein, 6 aa, synthetic construct
  REGION 1..6: note = HSA-1-CDR3
GGSLSR

SEQ ID NO: 116 - protein, 5 aa, synthetic construct
  REGION 1..5: note = HSA-2-CDR1
LNLMG

SEQ ID NO: 117 - protein, 16 aa, synthetic construct
  REGION 1..16: note = HSA-2-CDR2
TITVGDSTNYADSVKG

SEQ ID NO: 118 - protein, 8 aa, synthetic construct
  REGION 1..8: note = HSA-2-CDR3
RRTWHSEL

SEQ ID NO: 119 - protein, 5 aa, synthetic construct
  REGION 1..5: note = HSA-3-CDR1
INLLG

SEQ ID NO: 120 - protein, 16 aa, synthetic construct
  REGION 1..16: note = HSA-3-CDR2
TITVGDSTSYADSVKG

SEQ ID NO: 121 - protein, 8 aa, synthetic construct
  REGION 1..8: note = HSA-3-CDR3
RRTWHSEL

SEQ ID NO: 122 - protein, 5 aa, synthetic construct
  REGION 1..5: note = HSA-4-CDR1
SFGMS

SEQ ID NO: 123 - protein, 17 aa, synthetic construct
  REGION 1..17: note = HSA-4-CDR2
SINGRGDDTRYADSVKG

SEQ ID NO: 124 - protein, 7 aa, synthetic construct
  REGION 1..7: note = HSA-4-CDR3
GRSVSRS

SEQ ID NO: 125 - protein, 5 aa, synthetic construct
  REGION 1..5: note = HSA-5-CDR1
SFGMS

SEQ ID NO: 126 - protein, 17 aa, synthetic construct
  REGION 1..17: note = HSA-5-CDR2
AISADSSDKRYADSVKG

SEQ ID NO: 127 - protein, 5 aa, synthetic construct
  REGION 1..5: note = HSA-5-CDR3
GRGSP

SEQ ID NO: 128 - protein, 5 aa, synthetic construct
  REGION 1..5: note = HSA-6-CDR1
SFGMS

SEQ ID NO: 129 - protein, 17 aa, synthetic construct
  REGION 1..17: note = HSA-6-CDR2
AISADSSDKRYADSVKG

SEQ ID NO: 130 - protein, 5 aa, synthetic construct
  REGION 1..5: note = HSA-6-CDR3
GRGSP

SEQ ID NO: 131 - protein, 5 aa, synthetic construct
  REGION 1..5: note = HSA-7-CDR1
NYWMY

SEQ ID NO: 132 - protein, 17 aa, synthetic construct
  REGION 1..17: note = HSA-7-CDR2
RISTGGGYSYYADSVKG

SEQ ID NO: 133 - protein, 13 aa, synthetic construct
  REGION 1..13: note = HSA-7-CDR3
DREAQVDTLDFDY

SEQ ID NO: 134 - protein, 12 aa, synthetic construct
  REGION 1..12: note = DLL3-VL-1-CDR1
RASQRVNNNYLA

SEQ ID NO: 135 - protein, 7 aa, synthetic construct
  REGION 1..7: note = DLL3-VL-1-CDR2
GASSRAT

SEQ ID NO: 136 - protein, 9 aa, synthetic construct
  REGION 1..9: note = DLL3-VL-1-CDR3
QQYDRSPLT

SEQ ID NO: 137 - protein, 12 aa, synthetic construct
  REGION 1..12: note = DLL3-VL-2-CDR1
RASQSVNKNYLA

SEQ ID NO: 138 - protein, 7 aa, synthetic construct
  REGION 1..7: note = DLL3-VL-2-CDR2
GASSRAT

SEQ ID NO: 139 - protein, 9 aa, synthetic construct
  REGION 1..9: note = DLL3-VL-2-CDR3
QQYDRSPLT

SEQ ID NO: 140 - protein, 12 aa, synthetic construct
  REGION 1..12: note = DLL3-VL-3-CDR1
RASQSVSRGYLA

SEQ ID NO: 141 - protein, 7 aa, synthetic construct
  REGION 1..7: note = DLL3-VL-3-CDR2
GASSRAT

SEQ ID NO: 142 - protein, 9 aa, synthetic construct
  REGION 1..9: note = DLL3-VL-3-CDR3
QQYDTSPIT

SEQ ID NO: 143 - protein, 16 aa, synthetic construct
  REGION 1..16: note = DLL3-VL-4-CDR1
RSSQSLLHSNGYNYLD

SEQ ID NO: 144 - protein, 7 aa, synthetic construct
  REGION 1..7: note = DLL3-VL-4-CDR2
LGSNRAS

SEQ ID NO: 145 - protein, 9 aa, synthetic construct
  REGION 1..9: note = DLL3-VL-4-CDR3
MQALQTPFT

SEQ ID NO: 146 - protein, 11 aa, synthetic construct
  REGION 1..11: note = DLL3-VL-5-CDR1
RASQSISSYLN

SEQ ID NO: 147 - protein, 7 aa, synthetic construct
  REGION 1..7: note = DLL3-VL-5-CDR2
AASSLQS

SEQ ID NO: 148 - protein, 9 aa, synthetic construct
  REGION 1..9: note = DLL3-VL-5-CDR3
QQSFTTPLT

SEQ ID NO: 149 - protein, 11 aa, synthetic construct
  REGION 1..11: note = DLL3-VL-6-CDR1
SPGERATLSCK

SEQ ID NO: 150 - protein, 6 aa, synthetic construct
  REGION 1..6: note = DLL3-VL-6-CDR2
YASNRY

SEQ ID NO: 151 - protein, 9 aa, synthetic construct
  REGION 1..9: note = DLL3-VL-6-CDR3
QQDYTSPWT

SEQ ID NO: 152 - protein, 12 aa, synthetic construct
  REGION 1..12: note = DLL3-VL-7-CDR1
RASQSVSSSYLA

SEQ ID NO: 153 - protein, 7 aa, synthetic construct
  REGION 1..7: note = DLL3-VL-7-CDR2
GASTRAT

SEQ ID NO: 154 - protein, 9 aa, synthetic construct
  REGION 1..9: note = DLL3-VL-7-CDR3
QQYGTSPLT

SEQ ID NO: 155 - protein, 11 aa, synthetic construct
  REGION 1..11: note = DLL3-VL-8-CDR1
QASQDIRNYLN

SEQ ID NO: 156 - protein, 7 aa, synthetic construct
  REGION 1..7: note = DLL3-VL-8-CDR2
DASNLET

SEQ ID NO: 157 - protein, 10 aa, synthetic construct
  REGION 1..10: note = DLL3-VL-8-CDR3
QHYDNLPLTF

SEQ ID NO: 158 - protein, 11 aa, synthetic construct
  REGION 1..11: note = DLL3-VL-9-CDR1
RASQGIRNYLG

SEQ ID NO: 159 - protein, 7 aa, synthetic construct
  REGION 1..7: note = DLL3-VL-9-CDR2
AASSLQS

SEQ ID NO: 160 - protein, 10 aa, synthetic construct
  REGION 1..10: note = DLL3-VL-9-CDR3
LQHDSDLRTF

SEQ ID NO: 161 - protein, 10 aa, synthetic construct
  REGION 1..10: note = DLL3-VL-10-CDR1
TASSSVSSSY

SEQ ID NO: 162 - protein, 7 aa, synthetic construct
  REGION 1..7: note = DLL3-VL-10-CDR2
STSNLAS

SEQ ID NO: 163 - protein, 19 aa, synthetic construct
  REGION 1..19: note = DLL3-VL-10-CDR3
HQYHRSPFTFGAGTKLKIR

SEQ ID NO: 164 - protein, 9 aa, synthetic construct
  REGION 1..9: note = DLL3-VL-11-CDR1
RASQDISNY

SEQ ID NO: 165 - protein, 7 aa, synthetic construct
  REGION 1..7: note = DLL3-VL-11-CDR2
YTSRLHS

SEQ ID NO: 166 - protein, 19 aa, synthetic construct
  REGION 1..19: note = DLL3-VL-11-CDR3
QQGDMLPWTFGGGTKLEIK

SEQ ID NO: 167 - protein, 8 aa, synthetic construct
  REGION 1..8: note = DLL3-VL-12-CDR1
SASSSVSY

SEQ ID NO: 168 - protein, 7 aa, synthetic construct
  REGION 1..7: note = DLL3-VL-12-CDR2
DTSKLAS

SEQ ID NO: 169 - protein, 19 aa, synthetic construct
  REGION 1..19: note = DLL3-VL-12-CDR3
QQWTRNPLTFGAGTKLELK

SEQ ID NO: 170 - protein, 16 aa, synthetic construct
  REGION 1..16: note = DLL3-VL-13-CDR1
KSSQSVLVYSSNQKNY

SEQ ID NO: 171 - protein, 7 aa, synthetic construct
  REGION 1..7: note = DLL3-VL-13-CDR2
WASTRES

SEQ ID NO: 172 - protein, 18 aa, synthetic construct
  REGION 1..18: note = DLL3-VL-13-CDR3
HQYLSSWTFGGGTKLEIK

SEQ ID NO: 173 - protein, 9 aa, synthetic construct
  REGION 1..9: note = DLL3-VL-14-CDR1
QATQDIVKN

SEQ ID NO: 174 - protein, 7 aa, synthetic construct
  REGION 1..7: note = DLL3-VL-14-CDR2
YAIELAE

SEQ ID NO: 175 - protein, 19 aa, synthetic construct
  REGION 1..19: note = DLL3-VL-14-CDR3
LQFYEFPFTFGAGTKLELK

SEQ ID NO: 176 - protein, 10 aa, synthetic construct
  REGION 1..10: note = DLL3-VL-15-CDR1
TASSSVSSSY

SEQ ID NO: 177 - protein, 14 aa, synthetic construct
  REGION 1..14: note = DLL3-VL-16-CDR1
KSSQSLSDSDGKTY

SEQ ID NO: 178 - protein, 19 aa, synthetic construct
  REGION 1..19: note = DLL3-VL-15-CDR3
HQYHRSPFTFGSGTKLEIK

SEQ ID NO: 179 - protein, 19 aa, synthetic construct
  REGION 1..19: note = DLL3-VL-16-CDR3
WQGKHFPWTFGGGTKLEIK

SEQ ID NO: 180 - protein, 8 aa, synthetic construct
  REGION 1..8: note = DLL3-VL-17-CDR1
SASSSVSY

SEQ ID NO: 181 - protein, 7 aa, synthetic construct
  REGION 1..7: note = DLL3-VL-17-CDR2
LTSNLAS

SEQ ID NO: 182 - protein, 19 aa, synthetic construct
  REGION 1..19: note = DLL3-VL-17-CDR3
QQWRSNPFTFGSGTKLEIK

SEQ ID NO: 183 - protein, 9 aa, synthetic construct
  REGION 1..9: note = DLL3-VL-18-CDR1
RASENIYYN

SEQ ID NO: 184 - protein, 7 aa, synthetic construct
  REGION 1..7: note = DLL3-VL-18-CDR2
TANSLED

SEQ ID NO: 185 - protein, 19 aa, synthetic construct
  REGION 1..19: note = DLL3-VL-18-CDR3
KQAYDVPPTFGGGTKLEIK

SEQ ID NO: 186 - protein, 9 aa, synthetic construct
  REGION 1..9: note = DLL3-VL-19-CDR1
RASQNIINY

SEQ ID NO: 187 - protein, 7 aa, synthetic construct
  REGION 1..7: note = DLL3-VL-19-CDR2
YTSRLHS

SEQ ID NO: 188 - protein, 20 aa, synthetic construct
  REGION 1..20: note = DLL3-VL-19-CDR3
QQYSERPYTFGGGTKLEIKR

SEQ ID NO: 189 - protein, 9 aa, synthetic construct
  REGION 1..9: note = DLL3-VL-20-CDR1
KASQDIHKY

SEQ ID NO: 190 - protein, 7 aa, synthetic construct
  REGION 1..7: note = DLL3-VL-20-CDR2
YTSTLQP

SEQ ID NO: 191 - protein, 19 aa, synthetic construct
  REGION 1..19: note = DLL3-VL-20-CDR3
LQYNNLYTFGGGTKLEIKR

SEQ ID NO: 192 - protein, 9 aa, synthetic construct
  REGION 1..9: note = DLL3-VL-21-CDR1
QATQDIVKN

SEQ ID NO: 193 - protein, 7 aa, synthetic construct
  REGION 1..7: note = DLL3-VL-21-CDR2
YATELAE

SEQ ID NO: 194 - protein, 19 aa, synthetic construct
  REGION 1..19: note = DLL3-VL-21-CDR3
LQFYEFPFTFGAGTKLELK

SEQ ID NO: 195 - protein, 15 aa, synthetic construct
  REGION 1..15: note = DLL3-VL-22-CDR1
KSSQSLLNSSNQKNY

SEQ ID NO: 196 - protein, 7 aa, synthetic construct
  REGION 1..7: note = DLL3-VL-22-CDR2
FASTRES

SEQ ID NO: 197 - protein, 19 aa, synthetic construct
  REGION 1..19: note = DLL3-VL-22-CDR3
QQHYSIPLTFGAGTKLELK

SEQ ID NO: 198 - protein, 9 aa, synthetic construct
  REGION 1..9: note = DLL3-VL-23-CDR1
RASQDIKNY

SEQ ID NO: 199 - protein, 7 aa, synthetic construct
  REGION 1..7: note = DLL3-VL-23-CDR2
YTSRVHS

SEQ ID NO: 200 - protein, 17 aa, synthetic construct
  REGION 1..17: note = DLL3-VL-23-CDR3
QQGYTLPFTFGSGTKLE

SEQ ID NO: 201 - protein, 10 aa, synthetic construct
  REGION 1..10: note = DLL3-VL-24-CDR1
SASSSVSSRY

SEQ ID NO: 202 - protein, 7 aa, synthetic construct
  REGION 1..7: note = DLL3-VL-24-CDR2
STSNLAS

SEQ ID NO: 203 - protein, 19 aa, synthetic construct
  REGION 1..19: note = DLL3-VL-24-CDR3
HQWSNYPLTFGAGTKLELK

SEQ ID NO: 204 - protein, 8 aa, synthetic construct
  REGION 1..8: note = DLL3-VL-25-CDR1
SASSSVSY

SEQ ID NO: 205 - protein, 7 aa, synthetic construct
  REGION 1..7: note = DLL3-VL-25-CDR2
DSSKLAS

SEQ ID NO: 206 - protein, 19 aa, synthetic construct
  REGION 1..19: note = DLL3-VL-25-CDR3
QQWSSNPLTFGAGTKLELK

SEQ ID NO: 207 - protein, 14 aa, synthetic construct
  REGION 1..14: note = DLL3-VL-26-CDR1
KSSQSLSDSDGKTY

SEQ ID NO: 208 - protein, 7 aa, synthetic construct
  REGION 1..7: note = DLL3-VL-26-CDR2
LVSKLDS

SEQ ID NO: 209 - protein, 19 aa, synthetic construct
  REGION 1..19: note = DLL3-VL-26-CDR3
WQGKHFPWTFGGGTKLEIK

SEQ ID NO: 210 - protein, 8 aa, synthetic construct
  REGION 1..8: note = DLL3-VL-27-CDR1
SASSSVSY

SEQ ID NO: 211 - protein, 7 aa, synthetic construct
  REGION 1..7: note = DLL3-VL-27-CDR2
TTSNLAS

SEQ ID NO: 212 - protein, 19 aa, synthetic construct
  REGION 1..19: note = DLL3-VL-27-CDR3
QQRSLYPYTFGGGTKVEIK

SEQ ID NO: 213 - protein, 10 aa, synthetic construct
  REGION 1..10: note = DLL3-VL-28-CDR1
TASSSVTSSY

SEQ ID NO: 214 - protein, 7 aa, synthetic construct
  REGION 1..7: note = DLL3-VL-28-CDR2
STSNLAS

SEQ ID NO: 215 - protein, 19 aa, synthetic construct
  REGION 1..19: note = DLL3-VL-28-CDR3
HQFHRSPFTFGSGTKLEIK

SEQ ID NO: 216 - protein, 14 aa, synthetic construct
  REGION 1..14: note = DLL3-VL-29-CDR1
KSTKSLLNSDGFTY

SEQ ID NO: 217 - protein, 7 aa, synthetic construct
  REGION 1..7: note = DLL3-VL-29-CDR2
LVSNRFS

SEQ ID NO: 218 - protein, 19 aa, synthetic construct
  REGION 1..19: note = DLL3-VL-29-CDR3
FQSNYLPLTFGAGTKLELR

SEQ ID NO: 219 - protein, 9 aa, synthetic construct
  REGION 1..9: note = DLL3-VL-30-CDR1
KASQSVSND

SEQ ID NO: 220 - protein, 7 aa, synthetic construct
  REGION 1..7: note = DLL3-VL-30-CDR2
YASNRYS

SEQ ID NO: 221 - protein, 19 aa, synthetic construct
  REGION 1..19: note = DLL3-VL-30-CDR3
QQDYSSPWTFGGGTKLEIK

SEQ ID NO: 222 - protein, 9 aa, synthetic construct
  REGION 1..9: note = DLL3-VL-31-CDR1
RASQDISNY

SEQ ID NO: 223 - protein, 7 aa, synthetic construct
  REGION 1..7: note = DLL3-VL-31-CDR2
YTSRLHS

SEQ ID NO: 224 - protein, 19 aa, synthetic construct
  REGION 1..19: note = DLL3-VL-31-CDR3
QQGNTLPYTFGGGTKLEIK

SEQ ID NO: 225 - protein, 9 aa, synthetic construct
  REGION 1..9: note = DLL3-VL-32-CDR1
ITTPDIDDD

SEQ ID NO: 226 - protein, 7 aa, synthetic construct
  REGION 1..7: note = DLL3-VL-32-CDR2
EGNSLRP

SEQ ID NO: 227 - protein, 19 aa, synthetic construct
  REGION 1..19: note = DLL3-VL-32-CDR3
LQSDNMPFTFGSGTKLEIK

SEQ ID NO: 228 - protein, 8 aa, synthetic construct
  REGION 1..8: note = DLL3-VL-33-CDR1
SASSSINY

SEQ ID NO: 229 - protein, 7 aa, synthetic construct
  REGION 1..7: note = DLL3-VL-33-CDR2
DTSKLAS

SEQ ID NO: 230 - protein, 17 aa, synthetic construct
  REGION 1..17: note = DLL3-VL-33-CDR3
HQRSTWTFGGGTKLEIK

SEQ ID NO: 231 - protein, 9 aa, synthetic construct
  REGION 1..9: note = DLL3-VL-34-CDR1
RASQDVINY

SEQ ID NO: 232 - protein, 7 aa, synthetic construct
  REGION 1..7: note = DLL3-VL-34-CDR2
YTSRLHS

SEQ ID NO: 233 - protein, 20 aa, synthetic construct
  REGION 1..20: note = DLL3-VL-34-CDR3
QQYSERPYTFGGGTKLEIKR

SEQ ID NO: 234 - protein, 14 aa, synthetic construct
  REGION 1..14: note = DLL3-VL-35-CDR1
RSSQNIVHSDRYTY

SEQ ID NO: 235 - protein, 7 aa, synthetic construct
  REGION 1..7: note = DLL3-VL-35-CDR2
GVSNRFS

SEQ ID NO: 236 - protein, 19 aa, synthetic construct
  REGION 1..19: note = DLL3-VL-35-CDR3
FQGTHVPYTFGGGTKLEIK

SEQ ID NO: 237 - protein, 9 aa, synthetic construct
  REGION 1..9: note = DLL3-VL-36-CDR1
QATQDIVKN

SEQ ID NO: 238 - protein, 7 aa, synthetic construct
  REGION 1..7: note = DLL3-VL-36-CDR2
YATELAE

SEQ ID NO: 239 - protein, 19 aa, synthetic construct
  REGION 1..19: note = DLL3-VL-36-CDR3
LQFYEFPFTFGAGTKLELK

SEQ ID NO: 240 - protein, 14 aa, synthetic construct
  REGION 1..14: note = DLL3-VL-37-CDR1
KSTKSLLNSDGFTY

SEQ ID NO: 241 - protein, 7 aa, synthetic construct
  REGION 1..7: note = DLL3-VL-37-CDR2
LVSNRFS

SEQ ID NO: 242 - protein, 19 aa, synthetic construct
  REGION 1..19: note = DLL3-VL-37-CDR3
FQSNYLPLTFGAGTKLELR

SEQ ID NO: 243 - protein, 10 aa, synthetic construct
  REGION 1..10: note = DLL3-VL-38-CDR1
KASQDINSYL

SEQ ID NO: 244 - protein, 7 aa, synthetic construct
  REGION 1..7: note = DLL3-VL-38-CDR2
RANRLVD

SEQ ID NO: 245 - protein, 19 aa, synthetic construct
  REGION 1..19: note = DLL3-VL-38-CDR3
LQYDEFPLTFGAGTKLELK

SEQ ID NO: 246 - protein, 9 aa, synthetic construct
  REGION 1..9: note = DLL3-VL-39-CDR1
RASQDISNY

SEQ ID NO: 247 - protein, 7 aa, synthetic construct
  REGION 1..7: note = DLL3-VL-39-CDR2
YTSRLHS

SEQ ID NO: 248 - protein, 18 aa, synthetic construct
  REGION 1..18: note = DLL3-VL-39-CDR3
QQGNTLRTFGGGTKLEIK

SEQ ID NO: 249 - protein, 9 aa, synthetic construct
  REGION 1..9: note = DLL3-VL-40-CDR1
RASQGIRGT

SEQ ID NO: 250 - protein, 7 aa, synthetic construct
  REGION 1..7: note = DLL3-VL-40-CDR2
STSNLNS

SEQ ID NO: 251 - protein, 19 aa, synthetic construct
  REGION 1..19: note = DLL3-VL-40-CDR3
LQRNAYPLTFGAGTKLELK

SEQ ID NO: 252 - protein, 9 aa, synthetic construct
  REGION 1..9: note = DLL3-VL-41-CDR1
KASQDINSY

SEQ ID NO: 253 - protein, 7 aa, synthetic construct
  REGION 1..7: note = DLL3-VL-41-CDR2
RANRLVD

SEQ ID NO: 254 - protein, 20 aa, synthetic construct
  REGION 1..20: note = DLL3-VL-41-CDR3
LQYDEFPYTFGGGTKLEIKR

SEQ ID NO: 255 - protein, 9 aa, synthetic construct
  REGION 1..9: note = DLL3-VL-42-CDR1
KASQSVSND

SEQ ID NO: 256 - protein, 7 aa, synthetic construct
  REGION 1..7: note = DLL3-VL-42-CDR2
YASNRYT

SEQ ID NO: 257 - protein, 19 aa, synthetic construct
  REGION 1..19: note = DLL3-VL-42-CDR3
QQDYTSPWTFGGGTKLEIR

SEQ ID NO: 258 - protein, 9 aa, synthetic construct
  REGION 1..9: note = DLL3-VL-43-CDR1
KASQDVSIF

SEQ ID NO: 259 - protein, 7 aa, synthetic construct
  REGION 1..7: note = DLL3-VL-43-CDR2
SASYRYT

SEQ ID NO: 260 - protein, 19 aa, synthetic construct
  REGION 1..19: note = DLL3-VL-43-CDR3
QQHYGTPFTFGSGTKLKIR

SEQ ID NO: 261 - protein, 9 aa, synthetic construct
  REGION 1..9: note = DLL3-VL-44-CDR1
RASENIYSY

SEQ ID NO: 262 - protein, 7 aa, synthetic construct
  REGION 1..7: note = DLL3-VL-44-CDR2
NAKTLAE

SEQ ID NO: 263 - protein, 19 aa, synthetic construct
  REGION 1..19: note = DLL3-VL-44-CDR3
QHHYDSPLTFGAGTKLELR

SEQ ID NO: 264 - protein, 15 aa, synthetic construct
  REGION 1..15: note = DLL3-VL-45-CDR1
KSSQSLLNSSNQKNY

SEQ ID NO: 265 - protein, 7 aa, synthetic construct
  REGION 1..7: note = DLL3-VL-45-CDR2
FASTRES

SEQ ID NO: 266 - protein, 19 aa, synthetic construct
  REGION 1..19: note = DLL3-VL-45-CDR3
QQHYSIPLTFGAGTKLELK

SEQ ID NO: 267 - protein, 9 aa, synthetic construct
  REGION 1..9: note = DLL3-VL-46-CDR1
KASQDINSF

SEQ ID NO: 268 - protein, 7 aa, synthetic construct
  REGION 1..7: note = DLL3-VL-46-CDR2
RANRLVD

SEQ ID NO: 269 - protein, 20 aa, synthetic construct
  REGION 1..20: note = DLL3-VL-46-CDR3
LQYDEFPYTFGGGTKLEIKR

SEQ ID NO: 270 - protein, 8 aa, synthetic construct
  REGION 1..8: note = DLL3-VL-47-CDR1
SASSSVSY

SEQ ID NO: 271 - protein, 7 aa, synthetic construct
  REGION 1..7: note = DLL3-VL-47-CDR2
DTSKLAS

SEQ ID NO: 272 - protein, 19 aa, synthetic construct
  REGION 1..19: note = DLL3-VL-47-CDR3
QQWSSNPYTFGGGTKLEIK

SEQ ID NO: 273 - protein, 8 aa, synthetic construct
  REGION 1..8: note = DLL3-VL-48-CDR1
SVTSSVSY

SEQ ID NO: 274 - protein, 7 aa, synthetic construct
  REGION 1..7: note = DLL3-VL-48-CDR2
LTSNLAS

SEQ ID NO: 275 - protein, 19 aa, synthetic construct
  REGION 1..19: note = DLL3-VL-48-CDR3
QQWRNNPFTFGSGTKVEIK

SEQ ID NO: 276 - protein, 12 aa, synthetic construct
  REGION 1..12: note = DLL3-VL-49-CDR1
RSSTGAVTTSNY

SEQ ID NO: 277 - protein, 7 aa, synthetic construct
  REGION 1..7: note = DLL3-VL-49-CDR2
GTNNRAP

SEQ ID NO: 278 - protein, 19 aa, synthetic construct
  REGION 1..19: note = DLL3-VL-49-CDR3
GLWYSNHLVFGGGTKLTVL

SEQ ID NO: 279 - protein, 9 aa, synthetic construct
  REGION 1..9: note = DLL3-VL-50-CDR1
ITSTDIDDD

SEQ ID NO: 280 - protein, 7 aa, synthetic construct
  REGION 1..7: note = DLL3-VL-50-CDR2
EGNTLRP

SEQ ID NO: 281 - protein, 19 aa, synthetic construct
  REGION 1..19: note = DLL3-VL-50-CDR3
LQSDNMPLTFGAGTKLELK

SEQ ID NO: 282 - protein, 8 aa, synthetic construct
  REGION 1..8: note = DLL3-VL-51-CDR1
RASSSVNY

SEQ ID NO: 283 - protein, 7 aa, synthetic construct
  REGION 1..7: note = DLL3-VL-51-CDR2
YTSNLAP

SEQ ID NO: 284 - protein, 20 aa, synthetic construct
  REGION 1..20: note = DLL3-VL-51-CDR3
QQFTSSPYTFGGGTKLEIKR

SEQ ID NO: 285 - protein, 9 aa, synthetic construct
  REGION 1..9: note = DLL3-VL-52-CDR1
RASQDIGYS

SEQ ID NO: 286 - protein, 7 aa, synthetic construct
  REGION 1..7: note = DLL3-VL-52-CDR2
ATSSLDS

SEQ ID NO: 287 - protein, 19 aa, synthetic construct
  REGION 1..19: note = DLL3-VL-52-CDR3
LQYASSPWTFGGGTKLEIK

SEQ ID NO: 288 - protein, 9 aa, synthetic construct
  REGION 1..9: note = DLL3-VL-53-CDR1
KASQDINSY

SEQ ID NO: 289 - protein, 7 aa, synthetic construct
  REGION 1..7: note = DLL3-VL-53-CDR2
RANRLVD

SEQ ID NO: 290 - protein, 19 aa, synthetic construct
  REGION 1..19: note = DLL3-VL-53-CDR3
LQYDEFPFTFGSGTKLEIK

SEQ ID NO: 291 - protein, 15 aa, synthetic construct
  REGION 1..15: note = DLL3-VL-54-CDR1
KSSQSLLNSRTRKNY

SEQ ID NO: 292 - protein, 7 aa, synthetic construct
  REGION 1..7: note = DLL3-VL-54-CDR2
WASTRES

SEQ ID NO: 293 - protein, 19 aa, synthetic construct
  REGION 1..19: note = DLL3-VL-54-CDR3
KQSYNLYTFGGGTKLKIKR

SEQ ID NO: 294 - protein, 9 aa, synthetic construct
  REGION 1..9: note = DLL3-VL-55-CDR1
ITSTDIDDD

SEQ ID NO: 295 - protein, 7 aa, synthetic construct
  REGION 1..7: note = DLL3-VL-55-CDR2
EGNTLRP

SEQ ID NO: 296 - protein, 20 aa, synthetic construct
  REGION 1..20: note = DLL3-VL-55-CDR3
LKRDDLPYTFGGGTQVEIKR

SEQ ID NO: 297 - protein, 10 aa, synthetic construct
  REGION 1..10: note = DLL3-VL-56-CDR1
TASSSVSSSY

SEQ ID NO: 298 - protein, 7 aa, synthetic construct
  REGION 1..7: note = DLL3-VL-56-CDR2
STSNLAS

SEQ ID NO: 299 - protein, 19 aa, synthetic construct
  REGION 1..19: note = DLL3-VL-56-CDR3
HQYNRSPLTFGAGTKLELK

SEQ ID NO: 300 - protein, 9 aa, synthetic construct
  REGION 1..9: note = DLL3-VL-57-CDR1
KASQDIKKY

SEQ ID NO: 301 - protein, 7 aa, synthetic construct
  REGION 1..7: note = DLL3-VL-57-CDR2
YTSTLEP

SEQ ID NO: 302 - protein, 18 aa, synthetic construct
  REGION 1..18: note = DLL3-VL-57-CDR3
LQYDILWTFGGGTKLEIK

SEQ ID NO: 303 - protein, 10 aa, synthetic construct
  REGION 1..10: note = DLL3-VL-58-CDR1
SASSSVSSSY

SEQ ID NO: 304 - protein, 7 aa, synthetic construct
  REGION 1..7: note = DLL3-VL-58-CDR2
RTSNLAS

SEQ ID NO: 305 - protein, 19 aa, synthetic construct
  REGION 1..19: note = DLL3-VL-58-CDR3
RQWSGYPWTFGGGTKLEIK

SEQ ID NO: 306 - protein, 10 aa, synthetic construct
  REGION 1..10: note = DLL3-VL-59-CDR1
TASSSVSSSY

SEQ ID NO: 307 - protein, 7 aa, synthetic construct
  REGION 1..7: note = DLL3-VL-59-CDR2
STSNLAS

SEQ ID NO: 308 - protein, 19 aa, synthetic construct
  REGION 1..19: note = DLL3-VL-59-CDR3
HQYHRSPFTFGSGTKLEIK

SEQ ID NO: 309 - protein, 13 aa, synthetic construct
  REGION 1..13: note = DLL3-VL-60-CDR1
RASKSVSTSGYSY

SEQ ID NO: 310 - protein, 7 aa, synthetic construct
  REGION 1..7: note = DLL3-VL-60-CDR2
LASNLES

SEQ ID NO: 311 - protein, 19 aa, synthetic construct
  REGION 1..19: note = DLL3-VL-60-CDR3
QHSRELPLTFGAGTKLELK

SEQ ID NO: 312 - protein, 8 aa, synthetic construct
  REGION 1..8: note = DLL3-VL-61-CDR1
RASSSVSY

SEQ ID NO: 313 - protein, 7 aa, synthetic construct
  REGION 1..7: note = DLL3-VL-61-CDR2
ATSNLAS

SEQ ID NO: 314 - protein, 19 aa, synthetic construct
  REGION 1..19: note = DLL3-VL-61-CDR3
QQWSSNPPTFGAGTKLELK

SEQ ID NO: 315 - protein, 9 aa, synthetic construct
  REGION 1..9: note = DLL3-VL-62-CDR1
KASQDVGTA

SEQ ID NO: 316 - protein, 7 aa, synthetic construct
  REGION 1..7: note = DLL3-VL-62-CDR2
WASIRHT

SEQ ID NO: 317 - protein, 19 aa, synthetic construct
  REGION 1..19: note = DLL3-VL-62-CDR3
QQYSSYPLTFGAGTKLELK

SEQ ID NO: 318 - protein, 9 aa, synthetic construct
  REGION 1..9: note = DLL3-VL-63-CDR1
KASQDINSY

SEQ ID NO: 319 - protein, 7 aa, synthetic construct
  REGION 1..7: note = DLL3-VL-63-CDR2
RANRLVD

SEQ ID NO: 320 - protein, 19 aa, synthetic construct
  REGION 1..19: note = DLL3-VL-63-CDR3
LQYDEFPFTFGSGTKLEIK

SEQ ID NO: 321 - protein, 9 aa, synthetic construct
  REGION 1..9: note = DLL3-VL-64-CDR1
KASQDVNTA

SEQ ID NO: 322 - protein, 7 aa, synthetic construct
  REGION 1..7: note = DLL3-VL-64-CDR2
SASYRYT

SEQ ID NO: 323 - protein, 20 aa, synthetic construct
  REGION 1..20: note = DLL3-VL-64-CDR3
QQHYSSPYTFGGGTKLEIKR

SEQ ID NO: 324 - protein, 9 aa, synthetic construct
  REGION 1..9: note = DLL3-VL-65-CDR1
RASENIYSY

SEQ ID NO: 325 - protein, 7 aa, synthetic construct
  REGION 1..7: note = DLL3-VL-65-CDR2
NAKTLAE

SEQ ID NO: 326 - protein, 20 aa, synthetic construct
  REGION 1..20: note = DLL3-VL-65-CDR3
QHHYGTPYTFGGGTKLEIKR

SEQ ID NO: 327 - protein, 8 aa, synthetic construct
  REGION 1..8: note = DLL3-VL-66-CDR1
SASSSVSY

SEQ ID NO: 328 - protein, 7 aa, synthetic construct
  REGION 1..7: note = DLL3-VL-66-CDR2
DTSNLAS

SEQ ID NO: 329 - protein, 19 aa, synthetic construct
  REGION 1..19: note = DLL3-VL-66-CDR3
QEWSGNPLTFGDGTKLELK

SEQ ID NO: 330 - protein, 9 aa, synthetic construct
  REGION 1..9: note = DLL3-VL-67-CDR1
KASQSVSND

SEQ ID NO: 331 - protein, 7 aa, synthetic construct
  REGION 1..7: note = DLL3-VL-67-CDR2
YASNRYT

SEQ ID NO: 332 - protein, 19 aa, synthetic construct
  REGION 1..19: note = DLL3-VL-67-CDR3
QQDYSSPPTFGGGTKLEIK

SEQ ID NO: 333 - protein, 9 aa, synthetic construct
  REGION 1..9: note = DLL3-VL-68-CDR1
RASENIYYS

SEQ ID NO: 334 - protein, 7 aa, synthetic construct
  REGION 1..7: note = DLL3-VL-68-CDR2
NANSLED

SEQ ID NO: 335 - protein, 19 aa, synthetic construct
  REGION 1..19: note = DLL3-VL-68-CDR3
KQTYDVPLTFGAGTKLELK

SEQ ID NO: 336 - protein, 14 aa, synthetic construct
  REGION 1..14: note = DLL3-VL-69-CDR1
KSSQSLLDSDGTTY

SEQ ID NO: 337 - protein, 7 aa, synthetic construct
  REGION 1..7: note = DLL3-VL-69-CDR2
LVSKLDS

SEQ ID NO: 338 - protein, 19 aa, synthetic construct
  REGION 1..19: note = DLL3-VL-69-CDR3
WQGTHFPLTFGAGTKLELK

SEQ ID NO: 339 - protein, 8 aa, synthetic construct
  REGION 1..8: note = DLL3-VL-70-CDR1
RASSSVSY

SEQ ID NO: 340 - protein, 7 aa, synthetic construct
  REGION 1..7: note = DLL3-VL-70-CDR2
ATSNLAS

SEQ ID NO: 341 - protein, 20 aa, synthetic construct
  REGION 1..20: note = DLL3-VL-70-CDR3
QQWSSNPYTFGGGTKLEIKR

SEQ ID NO: 342 - protein, 14 aa, synthetic construct
  REGION 1..14: note = DLL3-VL-71-CDR1
KSSQSLLDSDGTTY

SEQ ID NO: 343 - protein, 7 aa, synthetic construct
  REGION 1..7: note = DLL3-VL-71-CDR2
LVSKLDS

SEQ ID NO: 344 - protein, 19 aa, synthetic construct
  REGION 1..19: note = DLL3-VL-71-CDR3
WQGTHFPLTFGAGTKLELK

SEQ ID NO: 345 - protein, 15 aa, synthetic construct
  REGION 1..15: note = DLL3-VL-72-CDR1
TSSQSLLTSGNQKNY

SEQ ID NO: 346 - protein, 7 aa, synthetic construct
  REGION 1..7: note = DLL3-VL-72-CDR2
WASTRES

SEQ ID NO: 347 - protein, 17 aa, synthetic construct
  REGION 1..17: note = DLL3-VL-72-CDR3
QNDYSLTFGAGTKLELK

SEQ ID NO: 348 - protein, 9 aa, synthetic construct
  REGION 1..9: note = DLL3-VL-73-CDR1
HVSQNINVW

SEQ ID NO: 349 - protein, 7 aa, synthetic construct
  REGION 1..7: note = DLL3-VL-73-CDR2
KASNLHT

SEQ ID NO: 350 - protein, 19 aa, synthetic construct
  REGION 1..19: note = DLL3-VL-73-CDR3
QQGQSYPFTFGSGTKLEIK

SEQ ID NO: 351 - protein, 13 aa, synthetic construct
  REGION 1..13: note = DLL3-VL-74-CDR1
KASQSVDYDGDSY

SEQ ID NO: 352 - protein, 7 aa, synthetic construct
  REGION 1..7: note = DLL3-VL-74-CDR2
AASNLES

SEQ ID NO: 353 - protein, 20 aa, synthetic construct
  REGION 1..20: note = DLL3-VL-74-CDR3
QQSNEDPYTFGGGTKLEIKR

SEQ ID NO: 354 - protein, 15 aa, synthetic construct
  REGION 1..15: note = DLL3-VL-75-CDR1
KSSQSLLYSSTQKNY

SEQ ID NO: 355 - protein, 7 aa, synthetic construct
  REGION 1..7: note = DLL3-VL-75-CDR2
WASTRES

SEQ ID NO: 356 - protein, 20 aa, synthetic construct
  REGION 1..20: note = DLL3-VL-75-CDR3
QQYYSYPYTFGGGTKLEIKR

SEQ ID NO: 357 - protein, 8 aa, synthetic construct
  REGION 1..8: note = DLL3-VL-76-CDR1
SASSSVSY

SEQ ID NO: 358 - protein, 7 aa, synthetic construct
  REGION 1..7: note = DLL3-VL-76-CDR2
STSNLAS

SEQ ID NO: 359 - protein, 20 aa, synthetic construct
  REGION 1..20: note = DLL3-VL-76-CDR3
QQRSSYPPTFGGGTKLEIKR

SEQ ID NO: 360 - protein, 9 aa, synthetic construct
  REGION 1..9: note = DLL3-VL-77-CDR1
KASQNVGTN

SEQ ID NO: 361 - protein, 7 aa, synthetic construct
  REGION 1..7: note = DLL3-VL-77-CDR2
SASYRYS

SEQ ID NO: 362 - protein, 19 aa, synthetic construct
  REGION 1..19: note = DLL3-VL-77-CDR3
QQYNSYPLTFGGGTKLEIK

SEQ ID NO: 363 - protein, 10 aa, synthetic construct
  REGION 1..10: note = DLL3-VL-78-CDR1
TASSSVSSSY

SEQ ID NO: 364 - protein, 7 aa, synthetic construct
  REGION 1..7: note = DLL3-VL-78-CDR2
STSNLAS

SEQ ID NO: 365 - protein, 19 aa, synthetic construct
  REGION 1..19: note = DLL3-VL-78-CDR3
HQYHRSPFTFGSGTKLEIK

SEQ ID NO: 366 - protein, 9 aa, synthetic construct
  REGION 1..9: note = DLL3-VL-79-CDR1
LASQTIGTW

SEQ ID NO: 367 - protein, 7 aa, synthetic construct
  REGION 1..7: note = DLL3-VL-79-CDR2
AATSLAD

SEQ ID NO: 368 - protein, 19 aa, synthetic construct
  REGION 1..19: note = DLL3-VL-79-CDR3
QQLYSTPWTFGGGTKLEIK

SEQ ID NO: 369 - protein, 9 aa, synthetic construct
  REGION 1..9: note = DLL3-VL-80-CDR1
HASQNINVW

SEQ ID NO: 370 - protein, 7 aa, synthetic construct
  REGION 1..7: note = DLL3-VL-80-CDR2
KASILHT

SEQ ID NO: 371 - protein, 19 aa, synthetic construct
  REGION 1..19: note = DLL3-VL-80-CDR3
QQGQSYPYTFGGGTKLEIK

SEQ ID NO: 372 - protein, 9 aa, synthetic construct
  REGION 1..9: note = DLL3-VL-81-CDR1
HASQNINVW

SEQ ID NO: 373 - protein, 7 aa, synthetic construct
  REGION 1..7: note = DLL3-VL-81-CDR2
KASNLHT

SEQ ID NO: 374 - protein, 19 aa, synthetic construct
  REGION 1..19: note = DLL3-VL-81-CDR3
QQGQSYPFTFGSGTKLEIK

SEQ ID NO: 375 - protein, 9 aa, synthetic construct
  REGION 1..9: note = DLL3-VL-82-CDR1
LASQTIGTW

SEQ ID NO: 376 - protein, 7 aa, synthetic construct
  REGION 1..7: note = DLL3-VL-82-CDR2
AATSLAD

SEQ ID NO: 377 - protein, 20 aa, synthetic construct
  REGION 1..20: note = DLL3-VL-82-CDR3
QQLYSTPYTFGGGTKLEIKR

SEQ ID NO: 378 - protein, 9 aa, synthetic construct
  REGION 1..9: note = DLL3-VL-83-CDR1
RASGSIHNY

SEQ ID NO: 379 - protein, 7 aa, synthetic construct
  REGION 1..7: note = DLL3-VL-83-CDR2
NAKTLVD

SEQ ID NO: 380 - protein, 19 aa, synthetic construct
  REGION 1..19: note = DLL3-VL-83-CDR3
QHFWTTPWTFGGGTKLEIK

SEQ ID NO: 381 - protein, 9 aa, synthetic construct
  REGION 1..9: note = DLL3-VL-84-CDR1
HVSQNINVW

SEQ ID NO: 382 - protein, 7 aa, synthetic construct
  REGION 1..7: note = DLL3-VL-84-CDR2
KASNLHT

SEQ ID NO: 383 - protein, 19 aa, synthetic construct
  REGION 1..19: note = DLL3-VL-84-CDR3
QQGQSYPFTFGSGTKLEIK

SEQ ID NO: 384 - protein, 9 aa, synthetic construct
  REGION 1..9: note = DLL3-VL-85-CDR1
LASQTIGTW

SEQ ID NO: 385 - protein, 7 aa, synthetic construct
  REGION 1..7: note = DLL3-VL-85-CDR2
AATSLAD

SEQ ID NO: 386 - protein, 19 aa, synthetic construct
  REGION 1..19: note = DLL3-VL-85-CDR3
QQLYSTPWTFGGGTKLEIK

SEQ ID NO: 387 - protein, 9 aa, synthetic construct
  REGION 1..9: note = DLL3-VL-86-CDR1
KASQSVSND

SEQ ID NO: 388 - protein, 7 aa, synthetic construct
  REGION 1..7: note = DLL3-VL-86-CDR2
CASNRYT

SEQ ID NO: 389 - protein, 19 aa, synthetic construct
  REGION 1..19: note = DLL3-VL-86-CDR3
QQDYSSPLTFGAGTKLELK

SEQ ID NO: 390 - protein, 13 aa, synthetic construct
  REGION 1..13: note = DLL3-VL-87-CDR1
KASQSVDHAGDSY

SEQ ID NO: 391 - protein, 7 aa, synthetic construct
  REGION 1..7: note = DLL3-VL-87-CDR2
AASNLES

SEQ ID NO: 392 - protein, 20 aa, synthetic construct
  REGION 1..20: note = DLL3-VL-87-CDR3
QQSNEDPYTFGGGTKLEIKR

SEQ ID NO: 393 - protein, 9 aa, synthetic construct
  REGION 1..9: note = DLL3-VL-88-CDR1
KASQDINRY

SEQ ID NO: 394 - protein, 7 aa, synthetic construct
  REGION 1..7: note = DLL3-VL-88-CDR2
RANRLVD

SEQ ID NO: 395 - protein, 19 aa, synthetic construct
  REGION 1..19: note = DLL3-VL-88-CDR3
LQYDEFPFTFGSGTKLEIK

SEQ ID NO: 396 - protein, 9 aa, synthetic construct
  REGION 1..9: note = DLL3-VL-89-CDR1
RASGNIHNY

SEQ ID NO: 397 - protein, 7 aa, synthetic construct
  REGION 1..7: note = DLL3-VL-89-CDR2
NAKTLAD

SEQ ID NO: 398 - protein, 19 aa, synthetic construct
  REGION 1..19: note = DLL3-VL-89-CDR3
QHFWSTPWTFGGGTKLEIK

SEQ ID NO: 399 - protein, 8 aa, synthetic construct
  REGION 1..8: note = DLL3-VL-90-CDR1
SASSSVSY

SEQ ID NO: 400 - protein, 7 aa, synthetic construct
  REGION 1..7: note = DLL3-VL-90-CDR2
STSNLAS

SEQ ID NO: 401 - protein, 19 aa, synthetic construct
  REGION 1..19: note = DLL3-VL-90-CDR3
HQWSSYHTFGGGTKLEIKR

SEQ ID NO: 402 - protein, 9 aa, synthetic construct
  REGION 1..9: note = DLL3-VL-91-CDR1
LASQTIGTW

SEQ ID NO: 403 - protein, 7 aa, synthetic construct
  REGION 1..7: note = DLL3-VL-91-CDR2
SATSLAD

SEQ ID NO: 404 - protein, 19 aa, synthetic construct
  REGION 1..19: note = DLL3-VL-91-CDR3
QQLYSTPWTFGGGTKLEIK

SEQ ID NO: 405 - protein, 9 aa, synthetic construct
  REGION 1..9: note = DLL3-VL-92-CDR1
KASQDVNTA

SEQ ID NO: 406 - protein, 7 aa, synthetic construct
  REGION 1..7: note = DLL3-VL-92-CDR2
SASYRYT

SEQ ID NO: 407 - protein, 19 aa, synthetic construct
  REGION 1..19: note = DLL3-VL-92-CDR3
QQHYSSPYTFGGGTKLEIK

SEQ ID NO: 408 - protein, 13 aa, synthetic construct
  REGION 1..13: note = DLL3-VL-93-CDR1
RASKSVSTSGYSY

SEQ ID NO: 409 - protein, 7 aa, synthetic construct
  REGION 1..7: note = DLL3-VL-93-CDR2
LASNLES

SEQ ID NO: 410 - protein, 20 aa, synthetic construct
  REGION 1..20: note = DLL3-VL-93-CDR3
QHSRELPFTFGGGTKLEIKR

SEQ ID NO: 411 - protein, 9 aa, synthetic construct
  REGION 1..9: note = DLL3-VL-94-CDR1
KASQDINSY

SEQ ID NO: 412 - protein, 7 aa, synthetic construct
  REGION 1..7: note = DLL3-VL-94-CDR2
RANRLVD

SEQ ID NO: 413 - protein, 19 aa, synthetic construct
  REGION 1..19: note = DLL3-VL-94-CDR3
LQYDEFPFTFGSGTKLEIK

SEQ ID NO: 414 - protein, 9 aa, synthetic construct
  REGION 1..9: note = DLL3-VL-95-CDR1
KASQDINNY

SEQ ID NO: 415 - protein, 7 aa, synthetic construct
  REGION 1..7: note = DLL3-VL-95-CDR2
RANRLVD

SEQ ID NO: 416 - protein, 20 aa, synthetic construct
  REGION 1..20: note = DLL3-VL-95-CDR3
LQYDEFPYTFGGGTKLEIKR

SEQ ID NO: 417 - protein, 14 aa, synthetic construct
  REGION 1..14: note = DLL3-VL-96-CDR1
RSSQSIVHSNGNTY

SEQ ID NO: 418 - protein, 7 aa, synthetic construct
  REGION 1..7: note = DLL3-VL-96-CDR2
KVSNRFS

SEQ ID NO: 419 - protein, 19 aa, synthetic construct
  REGION 1..19: note = DLL3-VL-96-CDR3
FQGSHVPLTFGAGTKLELK

SEQ ID NO: 420 - protein, 9 aa, synthetic construct
  REGION 1..9: note = DLL3-VL-97-CDR1
KASQSVSND

SEQ ID NO: 421 - protein, 7 aa, synthetic construct
  REGION 1..7: note = DLL3-VL-97-CDR2
YASNRYN

SEQ ID NO: 422 - protein, 19 aa, synthetic construct
  REGION 1..19: note = DLL3-VL-97-CDR3
QQDYSSPWTFGGGTKLEIK

SEQ ID NO: 423 - protein, 9 aa, synthetic construct
  REGION 1..9: note = DLL3-VL-98-CDR1
RASQDINNY

SEQ ID NO: 424 - protein, 7 aa, synthetic construct
  REGION 1..7: note = DLL3-VL-98-CDR2
YTSRLHS

SEQ ID NO: 425 - protein, 19 aa, synthetic construct
  REGION 1..19: note = DLL3-VL-98-CDR3
QQGDTLPWTFGGGTKLEIK

SEQ ID NO: 426 - protein, 8 aa, synthetic construct
  REGION 1..8: note = DLL3-VL-99-CDR1
SASSSVSY

SEQ ID NO: 427 - protein, 7 aa, synthetic construct
  REGION 1..7: note = DLL3-VL-99-CDR2
DTSNLAS

SEQ ID NO: 428 - protein, 19 aa, synthetic construct
  REGION 1..19: note = DLL3-VL-99-CDR3
QEWSNNPLTFGDGTKLELK

SEQ ID NO: 429 - protein, 9 aa, synthetic construct
  REGION 1..9: note = DLL3-VL-100-CDR1
HASQNINVW

SEQ ID NO: 430 - protein, 7 aa, synthetic construct
  REGION 1..7: note = DLL3-VL-100-CDR2
KASHLHT

SEQ ID NO: 431 - protein, 19 aa, synthetic construct
  REGION 1..19: note = DLL3-VL-100-CDR3
QQGQSYPFTFGSGTTLEIK

SEQ ID NO: 432 - protein, 15 aa, synthetic construct
  REGION 1..15: note = DLL3-VL-101-CDR1
MSSQSLLYSSTQKNY

SEQ ID NO: 433 - protein, 7 aa, synthetic construct
  REGION 1..7: note = DLL3-VL-101-CDR2
WASTRES

SEQ ID NO: 434 - protein, 20 aa, synthetic construct
  REGION 1..20: note = DLL3-VL-101-CDR3
QQYYSYPYTFGGGTKLEIKR

SEQ ID NO: 435 - protein, 8 aa, synthetic construct
  REGION 1..8: note = DLL3-VL-102-CDR1
SASSSVSY

SEQ ID NO: 436 - protein, 7 aa, synthetic construct
  REGION 1..7: note = DLL3-VL-102-CDR2
LTSNLAS

SEQ ID NO: 437 - protein, 20 aa, synthetic construct
  REGION 1..20: note = DLL3-VL-102-CDR3
QQWRSNPFTFGQGTKLEIKR

SEQ ID NO: 438 - protein, 9 aa, synthetic construct
  REGION 1..9: note = DLL3-VL-103-CDR1
RASENIYYN

SEQ ID NO: 439 - protein, 7 aa, synthetic construct
  REGION 1..7: note = DLL3-VL-103-CDR2
TANSLED

SEQ ID NO: 440 - protein, 19 aa, synthetic construct
  REGION 1..19: note = DLL3-VL-103-CDR3
KQAYDVPPTFGGGTKLEIK

SEQ ID NO: 441 - protein, 8 aa, synthetic construct
  REGION 1..8: note = DLL3-VL-104-CDR1
SASSSVSY

SEQ ID NO: 442 - protein, 7 aa, synthetic construct
  REGION 1..7: note = DLL3-VL-104-CDR2
DSSKLAS

SEQ ID NO: 443 - protein, 19 aa, synthetic construct
  REGION 1..19: note = DLL3-VL-104-CDR3
QQWSSNPLTFGQGTKLEIK

SEQ ID NO: 444 - protein, 9 aa, synthetic construct
  REGION 1..9: note = DLL3-VL-105-CDR1
KASQSVSND

SEQ ID NO: 445 - protein, 7 aa, synthetic construct
  REGION 1..7: note = DLL3-VL-105-CDR2
YASNRYS

SEQ ID NO: 446 - protein, 19 aa, synthetic construct
  REGION 1..19: note = DLL3-VL-105-CDR3
QQDYSSPWTFGGGTKVEIK

SEQ ID NO: 447 - protein, 9 aa, synthetic construct
  REGION 1..9: note = DLL3-VL-106-CDR1
KASQSVSND

SEQ ID NO: 448 - protein, 7 aa, synthetic construct
  REGION 1..7: note = DLL3-VL-106-CDR2
YASNRYT

SEQ ID NO: 449 - protein, 20 aa, synthetic construct
  REGION 1..20: note = DLL3-VL-106-CDR3
QQDYTSPWTFGQGTKLEIKR

SEQ ID NO: 450 - protein, 6 aa, synthetic construct
  REGION 1..6: note = CD16-VH-CDR1
TSYYMH

SEQ ID NO: 451 - protein, 17 aa, synthetic construct
  REGION 1..17: note = CD16-VH-CDR2
IINPSGGSTSYAQKFQG

SEQ ID NO: 452 - protein, 11 aa, synthetic construct
  REGION 1..11: note = CD16-VH-CDR3
GSAYYYDFADY

SEQ ID NO: 453 - protein, 11 aa, synthetic construct
  REGION 1..11: note = CD16-VL-1-CDR1
SGDKLEEKYVS

SEQ ID NO: 454 - protein, 7 aa, synthetic construct
  REGION 1..7: note = CD16-VL-1-CDR2
QDNKRPS

SEQ ID NO: 455 - protein, 9 aa, synthetic construct
  REGION 1..9: note = CD16-VL-1-CDR3
QVWDNYSVL

SEQ ID NO: 456 - protein, 11 aa, synthetic construct
  REGION 1..11: note = CD16-VL-2-CDR1
GGNNIESRNVH

SEQ ID NO: 457 - protein, 7 aa, synthetic construct
  REGION 1..7: note = CD16-VL-2-CDR2
RDNNRPS

SEQ ID NO: 458 - protein, 9 aa, synthetic construct
  REGION 1..9: note = CD16-VL-2-CDR3
QVWDNYTVL

SEQ ID NO: 459 - protein, 11 aa, synthetic construct
  REGION 1..11: note = CD16-VL-3-CDR1
GGNNIGSKNVH

SEQ ID NO: 460 - protein, 7 aa, synthetic construct
  REGION 1..7: note = CD16-VL-3-CDR2
RDSNRPS

SEQ ID NO: 461 - protein, 9 aa, synthetic construct
  REGION 1..9: note = CD16-VL-3-CDR3
QVWDNYIVL

SEQ ID NO: 462 - protein, 11 aa, synthetic construct
  REGION 1..11: note = CD16-VL-4-CDR1
EGNNIGSKNVH

SEQ ID NO: 463 - protein, 7 aa, synthetic construct
  REGION 1..7: note = CD16-VL-4-CDR2
DDSDRPS

SEQ ID NO: 464 - protein, 9 aa, synthetic construct
  REGION 1..9: note = CD16-VL-4-CDR3
QVWDNYSVL

SEQ ID NO: 465 - protein, 11 aa, synthetic construct
  REGION 1..11: note = CD16-VL-5-CDR1
GGNNIGSKNVH

SEQ ID NO: 466 - protein, 7 aa, synthetic construct
  REGION 1..7: note = CD16-VL-5-CDR2
RDSSRPS

SEQ ID NO: 467 - protein, 9 aa, synthetic construct
  REGION 1..9: note = CD16-VL-5-CDR3
QVWDDYIVV

SEQ ID NO: 468 - protein, 11 aa, synthetic construct
  REGION 1..11: note = CD16-VL-6-CDR1
GANDIGKRNVH

SEQ ID NO: 469 - protein, 7 aa, synthetic construct
  REGION 1..7: note = CD16-VL-6-CDR2
QDNKRPS

SEQ ID NO: 470 - protein, 9 aa, synthetic construct
  REGION 1..9: note = CD16-VL-6-CDR3
QVWDNYSVL

SEQ ID NO: 471 - protein, 11 aa, synthetic construct
  REGION 1..11: note = CD16-VL-7-CDR1
GGHNIGSKNVH

SEQ ID NO: 472 - protein, 7 aa, synthetic construct
  REGION 1..7: note = CD16-VL-7-CDR2
QDNKRPS

SEQ ID NO: 473 - protein, 9 aa, synthetic construct
  REGION 1..9: note = CD16-VL-7-CDR3
QVWDNYSVL

SEQ ID NO: 474 - protein, 14 aa, synthetic construct
  REGION 1..14: note = CD3-VL-1-CDR1
GSSTGAVTSGYYPN

SEQ ID NO: 475 - protein, 7 aa, synthetic construct
  REGION 1..7: note = CD3-VL-1-CDR2
GTKFLAP

SEQ ID NO: 476 - protein, 9 aa, synthetic construct
  REGION 1..9: note = CD3-VL-1-CDR3
ALWYSNRWV

SEQ ID NO: 477 - protein, 14 aa, synthetic construct
  REGION 1..14: note = CD3-VL-2-CDR1
RSSTGAVTSGYYPN

SEQ ID NO: 478 - protein, 7 aa, synthetic construct
  REGION 1..7: note = CD3-VL-2-CDR2
ATDMRPS

SEQ ID NO: 479 - protein, 9 aa, synthetic construct
  REGION 1..9: note = CD3-VL-2-CDR3
ALWYSNRWV

SEQ ID NO: 480 - protein, 14 aa, synthetic construct
  REGION 1..14: note = CD3-VL-3-CDR1
GSSTGAVTSGNYPN

SEQ ID NO: 481 - protein, 7 aa, synthetic construct
  REGION 1..7: note = CD3-VL-3-CDR2
GTKFLAP

SEQ ID NO: 482 - protein, 9 aa, synthetic construct
  REGION 1..9: note = CD3-VL-3-CDR3
VLWYSNRWV

SEQ ID NO: 483 - protein, 5 aa, synthetic construct
  REGION 1..5: note = CD3-VH-1-CDR1
IYAMN

SEQ ID NO: 484 - protein, 19 aa, synthetic construct
  REGION 1..19: note = CD3-VH-1-CDR2
RIRSKYNNYATYYADSVKS

SEQ ID NO: 485 - protein, 14 aa, synthetic construct
  REGION 1..14: note = CD3-VH-1-CDR3
HGNFGNSYVSFFAY

SEQ ID NO: 486 - protein, 5 aa, synthetic construct
  REGION 1..5: note = CD3-VH-2-CDR1
KYAMN

SEQ ID NO: 487 - protein, 19 aa, synthetic construct
  REGION 1..19: note = CD3-VH-2-CDR2
RIRSKYNNYATYYADSVKD

SEQ ID NO: 488 - protein, 14 aa, synthetic construct
  REGION 1..14: note = CD3-VH-2-CDR3
HGNFGNSYISYWAY

SEQ ID NO: 489 - protein, 5 aa, synthetic construct
  REGION 1..5: note = CD3-VH-3-CDR1
SYAMN

SEQ ID NO: 490 - protein, 19 aa, synthetic construct
  REGION 1..19: note = CD3-VH-3-CDR2
RIRSKYNNYATYYADSVKG

SEQ ID NO: 491 - protein, 14 aa, synthetic construct
  REGION 1..14: note = CD3-VH-3-CDR3
HGNFGNSYLSFWAY

SEQ ID NO: 492 - protein, 5 aa, synthetic construct
  REGION 1..5: note = CD3-VH-4-CDR1
RYAMN

SEQ ID NO: 493 - protein, 19 aa, synthetic construct
  REGION 1..19: note = CD3-VH-4-CDR2
RIRSKYNNYATYYADSVKG

SEQ ID NO: 494 - protein, 14 aa, synthetic construct
  REGION 1..14: note = CD3-VH-4-CDR3
HGNFGNSYLSYFAY

SEQ ID NO: 495 - protein, 5 aa, synthetic construct
  REGION 1..5: note = CD3-VH-5-CDR1
VYAMN

SEQ ID NO: 496 - protein, 19 aa, synthetic construct
  REGION 1..19: note = CD3-VH-5-CDR2
RIRSKYNNYATYYADSVKK

SEQ ID NO: 497 - protein, 14 aa, synthetic construct
  REGION 1..14: note = CD3-VH-5-CDR3
HGNFGNSYLSWWAY

SEQ ID NO: 498 - protein, 5 aa, synthetic construct
  REGION 1..5: note = CD3-VH-6-CDR1
KYAMN

SEQ ID NO: 499 - protein, 19 aa, synthetic construct
  REGION 1..19: note = CD3-VH-6-CDR2
RIRSKYNNYATYYADSVKS

SEQ ID NO: 500 - protein, 14 aa, synthetic construct
  REGION 1..14: note = CD3-VH-6-CDR3
HGNFGNSYTSYYAY

SEQ ID NO: 501 - protein, 5 aa, synthetic construct
  REGION 1..5: note = CD3-VH-7-CDR1
GYAMN

SEQ ID NO: 502 - protein, 19 aa, synthetic construct
  REGION 1..19: note = CD3-VH-7-CDR2
RIRSKYNNYATYYADSVKE

SEQ ID NO: 503 - protein, 14 aa, synthetic construct
  REGION 1..14: note = CD3-VH-7-CDR3
HRNFGNSYLSWFAY

SEQ ID NO: 504 - protein, 5 aa, synthetic construct
  REGION 1..5: note = CD3-VH-8-CDR1
VYAMN

SEQ ID NO: 505 - protein, 19 aa, synthetic construct
  REGION 1..19: note = CD3-VH-8-CDR2
RIRSKYNNYATYYADSVKK

SEQ ID NO: 506 - protein, 14 aa, synthetic construct
  REGION 1..14: note = CD3-VH-8-CDR3
HGNFGNSYISWWAY

SEQ ID NO: 507 - protein, 5 aa, synthetic construct
  REGION 1..5: note = CD3-VH-9-CDR1
SYAMN

SEQ ID NO: 508 - protein, 19 aa, synthetic construct
  REGION 1..19: note = CD3-VH-9-CDR2
RIRSKYNNYATYYADSVKG

SEQ ID NO: 509 - protein, 14 aa, synthetic construct
  REGION 1..14: note = CD3-VH-9-CDR3
HGNFGNSYVSWWAY

SEQ ID NO: 510 - protein, 5 aa, synthetic construct
  REGION 1..5: note = CD3-VH-10-CDR1
KYAMN

SEQ ID NO: 511 - protein, 19 aa, synthetic construct
  REGION 1..19: note = CD3-VH-10-CDR2
RIRSKYNNYATYYADSVKD

SEQ ID NO: 512 - protein, 14 aa, synthetic construct
  REGION 1..14: note = CD3-VH-10-CDR3
HGNFGNSYISYWAY

SEQ ID NO: 513 - protein, 13 aa, synthetic construct
  REGION 1..13: note = CD3-VL-4-CDR1
SSTGAVTTSNYAN

SEQ ID NO: 514 - protein, 6 aa, synthetic construct
  REGION 1..6: note = CD3-VL-4-CDR2
GTNKRA

SEQ ID NO: 515 - protein, 8 aa, synthetic construct
  REGION 1..8: note = CD3-VL-4-CDR3
LWYSNLWV

SEQ ID NO: 516 - protein, 5 aa, synthetic construct
  REGION 1..5: note = CD3-VH-11-CDR1
TYAMN

SEQ ID NO: 517 - protein, 18 aa, synthetic construct
  REGION 1..18: note = CD3-VH-11-CDR2
IRSKYNNYATYYADSVKD

SEQ ID NO: 518 - protein, 14 aa, synthetic construct
  REGION 1..14: note = CD3-VH-11-CDR3
HGNFGNSYVSWFAY

SEQ ID NO: 519 - protein, 8 aa, synthetic construct
  REGION 1..8: note = CD3-VH-12-CDR1
GFTFDDYS

SEQ ID NO: 520 - protein, 8 aa, synthetic construct
  REGION 1..8: note = CD3-VH-12-CDR2
ISWNSGSK

SEQ ID NO: 521 - protein, 17 aa, synthetic construct
  REGION 1..17: note = CD3-VH-12-CDR3
AKYGSGYGKFYHYGLDV

SEQ ID NO: 522 - protein, 8 aa, synthetic construct
  REGION 1..8: note = CD3-VH-13-CDR1
GFTFDDYA

SEQ ID NO: 523 - protein, 8 aa, synthetic construct
  REGION 1..8: note = CD3-VH-13-CDR2
ISWNSGSI

SEQ ID NO: 524 - protein, 17 aa, synthetic construct
  REGION 1..17: note = CD3-VH-13-CDR3
AKDGSGYGYFYYYGMDV

SEQ ID NO: 525 - protein, 8 aa, synthetic construct
  REGION 1..8: note = CD3-VH-14-CDR1
GFTFDDYS

SEQ ID NO: 526 - protein, 8 aa, synthetic construct
  REGION 1..8: note = CD3-VH-14-CDR2
ISWNSGSI

SEQ ID NO: 527 - protein, 17 aa, synthetic construct
  REGION 1..17: note = CD3-VH-14-CDR3
AKDGSGYGYFYYYGMDV

SEQ ID NO: 528 - protein, 8 aa, synthetic construct
  REGION 1..8: note = CD3-VH-15-CDR1
GFTFDDYA

SEQ ID NO: 529 - protein, 8 aa, synthetic construct
  REGION 1..8: note = CD3-VH-15-CDR2
ISWNSGSI

SEQ ID NO: 530 - protein, 17 aa, synthetic construct
  REGION 1..17: note = CD3-VH-15-CDR3
AKDGSGYGKFYYYGMDV

SEQ ID NO: 531 - protein, 8 aa, synthetic construct
  REGION 1..8: note = CD3-VH-16-CDR1
GFTFDDYS

SEQ ID NO: 532 - protein, 8 aa, synthetic construct
  REGION 1..8: note = CD3-VH-16-CDR2
ISWNSGSI

SEQ ID NO: 533 - protein, 17 aa, synthetic construct
  REGION 1..17: note = CD3-VH-16-CDR3
AKDGSGYGKFYYYGMDV

SEQ ID NO: 534 - protein, 8 aa, synthetic construct
  REGION 1..8: note = CD3-VH-17-CDR1
GFTFDDYS

SEQ ID NO: 535 - protein, 8 aa, synthetic construct
  REGION 1..8: note = CD3-VH-17-CDR2
ISWNSGSI

SEQ ID NO: 536 - protein, 17 aa, synthetic construct
  REGION 1..17: note = CD3-VH-17-CDR3
AKYGSGYGKFYHYGLDV

SEQ ID NO: 537 - protein, 8 aa, synthetic construct
  REGION 1..8: note = CD3-VH-18-CDR1
GFTFDDYS

SEQ ID NO: 538 - protein, 8 aa, synthetic construct
  REGION 1..8: note = CD3-VH-18-CDR2
ISWNSGSK

SEQ ID NO: 539 - protein, 17 aa, synthetic construct
  REGION 1..17: note = CD3-VH-18-CDR3
AKDGSGYGKFYHYGLDV

SEQ ID NO: 540 - protein, 8 aa, synthetic construct
  REGION 1..8: note = CD3-VH-19-CDR1
GFTFDDYS

SEQ ID NO: 541 - protein, 8 aa, synthetic construct
  REGION 1..8: note = CD3-VH-19-CDR2
ISWNSGSK

SEQ ID NO: 542 - protein, 17 aa, synthetic construct
  REGION 1..17: note = CD3-VH-19-CDR3
AKYGSGYGKFYYYGLDV

SEQ ID NO: 543 - protein, 8 aa, synthetic construct
  REGION 1..8: note = CD3-VH-20-CDR1
GFTFDDYS

SEQ ID NO: 544 - protein, 8 aa, synthetic construct
  REGION 1..8: note = CD3-VH-20-CDR2
ISWNSGSK

SEQ ID NO: 545 - protein, 17 aa, synthetic construct
  REGION 1..17: note = CD3-VH-20-CDR3
AKYGSGYGKFYHYGMDV

SEQ ID NO: 546 - protein, 8 aa, synthetic construct
  REGION 1..8: note = CD3-VH-21-CDR1
GFTFDDYS

SEQ ID NO: 547 - protein, 8 aa, synthetic construct
  REGION 1..8: note = CD3-VH-21-CDR2
ISWNSGSI

SEQ ID NO: 548 - protein, 17 aa, synthetic construct
  REGION 1..17: note = CD3-VH-21-CDR3
AKDGSGYGKFYHYGLDV

SEQ ID NO: 549 - protein, 8 aa, synthetic construct
  REGION 1..8: note = CD3-VH-22-CDR1
GFTFDDYS

SEQ ID NO: 550 - protein, 8 aa, synthetic construct
  REGION 1..8: note = CD3-VH-22-CDR2
ISWNSGSI

SEQ ID NO: 551 - protein, 17 aa, synthetic construct
  REGION 1..17: note = CD3-VH-22-CDR3
AKYGSGYGKFYYYGLDV

SEQ ID NO: 552 - protein, 8 aa, synthetic construct
  REGION 1..8: note = CD3-VH-23-CDR1
GFTFDDYS

SEQ ID NO: 553 - protein, 8 aa, synthetic construct
  REGION 1..8: note = CD3-VH-23-CDR2
ISWNSGSI

SEQ ID NO: 554 - protein, 17 aa, synthetic construct
  REGION 1..17: note = CD3-VH-23-CDR3
AKYGSGYGKFYHYGMDV

SEQ ID NO: 555 - protein, 8 aa, synthetic construct
  REGION 1..8: note = CD3-VH-24-CDR1
GFTFDDYS

SEQ ID NO: 556 - protein, 8 aa, synthetic construct
  REGION 1..8: note = CD3-VH-24-CDR2
ISWNSGSK

SEQ ID NO: 557 - protein, 17 aa, synthetic construct
  REGION 1..17: note = CD3-VH-24-CDR3
AKDGSGYGKFYYYGLDV

SEQ ID NO: 558 - protein, 8 aa, synthetic construct
  REGION 1..8: note = CD3-VH-25-CDR1
GFTFDDYS

SEQ ID NO: 559 - protein, 8 aa, synthetic construct
  REGION 1..8: note = CD3-VH-25-CDR2
ISWNSGSK

SEQ ID NO: 560 - protein, 17 aa, synthetic construct
  REGION 1..17: note = CD3-VH-25-CDR3
AKDGSGYGKFYHYGMDV

SEQ ID NO: 561 - protein, 8 aa, synthetic construct
  REGION 1..8: note = CD3-VH-26-CDR1
GFTFDDYS

SEQ ID NO: 562 - protein, 8 aa, synthetic construct
  REGION 1..8: note = CD3-VH-26-CDR2
ISWNSGSK

SEQ ID NO: 563 - protein, 17 aa, synthetic construct
  REGION 1..17: note = CD3-VH-26-CDR3
AKYGSGYGKFYYYGMDV

SEQ ID NO: 564 - protein, 8 aa, synthetic construct
  REGION 1..8: note = CD3-VH-27-CDR1
GFTFDDYS

SEQ ID NO: 565 - protein, 8 aa, synthetic construct
  REGION 1..8: note = CD3-VH-27-CDR2
ISWNSGSI

SEQ ID NO: 566 - protein, 17 aa, synthetic construct
  REGION 1..17: note = CD3-VH-27-CDR3
AKDGSGYGKFYYYGLDV

SEQ ID NO: 567 - protein, 8 aa, synthetic construct
  REGION 1..8: note = CD3-VH-28-CDR1
GFTFDDYS

SEQ ID NO: 568 - protein, 8 aa, synthetic construct
  REGION 1..8: note = CD3-VH-28-CDR2
ISWNSGSI

SEQ ID NO: 569 - protein, 17 aa, synthetic construct
  REGION 1..17: note = CD3-VH-28-CDR3
AKDGSGYGKFYHYGMDV

SEQ ID NO: 570 - protein, 8 aa, synthetic construct
  REGION 1..8: note = CD3-VH-29-CDR1
GFTFDDYS

SEQ ID NO: 571 - protein, 8 aa, synthetic construct
  REGION 1..8: note = CD3-VH-29-CDR2
ISWNSGSI

SEQ ID NO: 572 - protein, 17 aa, synthetic construct
  REGION 1..17: note = CD3-VH-29-CDR3
AKYGSGYGKFYYYGMDV

SEQ ID NO: 573 - protein, 8 aa, synthetic construct
  REGION 1..8: note = CD3-VH-30-CDR1
GFTFDDYS

SEQ ID NO: 574 - protein, 8 aa, synthetic construct
  REGION 1..8: note = CD3-VH-30-CDR2
ISWNSGSK

SEQ ID NO: 575 - protein, 17 aa, synthetic construct
  REGION 1..17: note = CD3-VH-30-CDR3
AKDGSGYGKFYYYGMDV

SEQ ID NO: 576 - protein, 8 aa, synthetic construct
  REGION 1..8: note = CD3-VH-31-CDR1
GFTFDDYS

SEQ ID NO: 577 - protein, 8 aa, synthetic construct
  REGION 1..8: note = CD3-VH-31-CDR2
ISWNSGSK

SEQ ID NO: 578 - protein, 17 aa, synthetic construct
  REGION 1..17: note = CD3-VH-31-CDR3
AKYGSGYGKFYHYGLDV

SEQ ID NO: 579 - protein, 5 aa, synthetic construct
  REGION 1..5: note = CD3-VH-32-CDR1
NYYIH

SEQ ID NO: 580 - protein, 17 aa, synthetic construct
  REGION 1..17: note = CD3-VH-32-CDR2
WIYPGDGNTKYNEKFKG

SEQ ID NO: 581 - protein, 10 aa, synthetic construct
  REGION 1..10: note = CD3-VH-32-CDR3
DSYSNYYFDY

SEQ ID NO: 582 - protein, 5 aa, synthetic construct
  REGION 1..5: note = CD3-VH-n-CDR1
SYYIH

SEQ ID NO: 583 - protein, 17 aa, synthetic construct
  REGION 1..17: note = CD3-VH-n-CDR2
WIYPENDNTKYNEKFKD

SEQ ID NO: 584 - protein, 10 aa, synthetic construct
  REGION 1..10: note = CD3-VH-n-CDR3
DGYSRYYFDY

SEQ ID NO: 585 - protein, 5 aa, synthetic construct
  REGION 1..5: note = CD3-VH-n-CDR1
GYTMN

SEQ ID NO: 586 - protein, 17 aa, synthetic construct
  REGION 1..17: note = CD3-VH-n-CDR2
LINPYKGVSTYNQKFKD

SEQ ID NO: 587 - protein, 10 aa, synthetic construct
  REGION 1..10: note = CD3-VH-n-CDR3
DAYSRYYFDY

SEQ ID NO: 588 - protein, 5 aa, synthetic construct
  REGION 1..5: note = CD3-VH-n-CDR1
TYAMN

SEQ ID NO: 589 - protein, 17 aa, synthetic construct
  REGION 1..17: note = CD3-VH-n-CDR2
LINPYKGVTTYADSVKG

SEQ ID NO: 590 - protein, 10 aa, synthetic construct
  REGION 1..10: note = CD3-VH-n-CDR3
TGYSRYYFDY

SEQ ID NO: 591 - protein, 5 aa, synthetic construct
  REGION 1..5: note = CD3-VH-n-CDR1
NYGMN

SEQ ID NO: 592 - protein, 17 aa, synthetic construct
  REGION 1..17: note = CD3-VH-n-CDR2
LINPYKGVSTYADSVKG

SEQ ID NO: 593 - protein, 10 aa, synthetic construct
  REGION 1..10: note = CD3-VH-n-CDR3
DGYSRYAFDY

SEQ ID NO: 594 - protein, 5 aa, synthetic construct
  REGION 1..5: note = CD3-VH-n-CDR1
SYYIH

SEQ ID NO: 595 - protein, 19 aa, synthetic construct
  REGION 1..19: note = CD3-VH-n-CDR2
RIRSKYNNYATYYADSVKD

SEQ ID NO: 596 - protein, 10 aa, synthetic construct
  REGION 1..10: note = CD3-VH-n-CDR3
SGYSRYYFDY

SEQ ID NO: 597 - protein, 5 aa, synthetic construct
  REGION 1..5: note = CD3-VH-n-CDR1
NYAIH

SEQ ID NO: 598 - protein, 17 aa, synthetic construct
  REGION 1..17: note = CD3-VH-n-CDR2
WINTNTGKPTYAEEFKG

SEQ ID NO: 599 - protein, 10 aa, synthetic construct
  REGION 1..10: note = CD3-VH-n-CDR3
DGYSRAYFDY

SEQ ID NO: 600 - protein, 10 aa, synthetic construct
  REGION 1..10: note = CD3-VH-n-CDR1
GFSLTNYAIH

SEQ ID NO: 601 - protein, 17 aa, synthetic construct
  REGION 1..17: note = CD3-VH-n-CDR2
WIYPGNVNTKYNEKFKG

SEQ ID NO: 602 - protein, 10 aa, synthetic construct
  REGION 1..10: note = CD3-VH-n-CDR3
DSYSNYYFDY

SEQ ID NO: 603 - protein, 5 aa, synthetic construct
  REGION 1..5: note = CD3-VH-n-CDR1
GSAMH

SEQ ID NO: 604 - protein, 16 aa, synthetic construct
  REGION 1..16: note = CD3-VH-n-CDR2
VIWAGGNTKYNSALMS

SEQ ID NO: 605 - protein, 13 aa, synthetic construct
  REGION 1..13: note = CD3-VH-n-CDR3
SGYYGDSDWYFDV

SEQ ID NO: 606 - protein, 5 aa, synthetic construct
  REGION 1..5: note = CD3-VH-n-CDR1
SDYIH

SEQ ID NO: 607 - protein, 17 aa, synthetic construct
  REGION 1..17: note = CD3-VH-n-CDR2
GVIWAGGNTKYNSALMS

SEQ ID NO: 608 - protein, 14 aa, synthetic construct
  REGION 1..14: note = CD3-VH-n-CDR3
HGNFGNSYVSWFAY

SEQ ID NO: 609 - protein, 5 aa, synthetic construct
  REGION 1..5: note = CD3-VH-n-CDR1
SHYLH

SEQ ID NO: 610 - protein, 19 aa, synthetic construct
  REGION 1..19: note = CD3-VH-n-CDR2
RIRSRANSYATAYAASVKD

SEQ ID NO: 611 - protein, 11 aa, synthetic construct
  REGION 1..11: note = CD3-VH-n-CDR3
RGDYRYAWFLY

SEQ ID NO: 612 - protein, 5 aa, synthetic construct
  REGION 1..5: note = CD3-VH-n-CDR1
SYYIH

SEQ ID NO: 613 - protein, 17 aa, synthetic construct
  REGION 1..17: note = CD3-VH-n-CDR2
WIYFGNVNTKYNEKFKG

SEQ ID NO: 614 - protein, 8 aa, synthetic construct
  REGION 1..8: note = CD3-VH-n-CDR3
NHDYYFDY

SEQ ID NO: 615 - protein, 5 aa, synthetic construct
  REGION 1..5: note = CD3-VH-n-CDR1
SYYIH

SEQ ID NO: 616 - protein, 17 aa, synthetic construct
  REGION 1..17: note = CD3-VH-n-CDR2
WINPGDGNVKYNEKFKD

SEQ ID NO: 617 - protein, 11 aa, synthetic construct
  REGION 1..11: note = CD3-VH-n-CDR3
EDSSGYVALDY

SEQ ID NO: 618 - protein, 5 aa, synthetic construct
  REGION 1..5: note = CD3-VH-n-CDR1
NYGMN

SEQ ID NO: 619 - protein, 17 aa, synthetic construct
  REGION 1..17: note = CD3-VH-n-CDR2
WIFPGSDNTKYNEKFKG

SEQ ID NO: 620 - protein, 28 aa, synthetic construct
  REGION 1..28: note = CD3-VH-n-CDR3
QMNSLRAEDTAVYYCAREDSSGYVALDY

SEQ ID NO: 621 - protein, 5 aa, synthetic construct
  REGION 1..5: note = CD3-VH-n-CDR1
NYYIH

SEQ ID NO: 622 - protein, 17 aa, synthetic construct
  REGION 1..17: note = CD3-VH-n-CDR2
WIYPGNVNTKYNEKFKG

SEQ ID NO: 623 - protein, 9 aa, synthetic construct
  REGION 1..9: note = CD3-VH-n-CDR3
DTMVRGIDY

SEQ ID NO: 624 - protein, 5 aa, synthetic construct
  REGION 1..5: note = CD3-VH-n-CDR1
SYWMH

SEQ ID NO: 625 - protein, 17 aa, synthetic construct
  REGION 1..17: note = CD3-VH-n-CDR2
WINTNTGKPTYAEDFKG

SEQ ID NO: 626 - protein, 8 aa, synthetic construct
  REGION 1..8: note = CD3-VH-n-CDR3
DGGYYFDY

SEQ ID NO: 627 - protein, 5 aa, synthetic construct
  REGION 1..5: note = CD3-VH-n-CDR1
NYYMH

SEQ ID NO: 628 - protein, 17 aa, synthetic construct
  REGION 1..17: note = CD3-VH-n-CDR2
WIYPGSDNTKFNDKFKG

SEQ ID NO: 629 - protein, 8 aa, synthetic construct
  REGION 1..8: note = CD3-VH-n-CDR3
DGAYYFDY

SEQ ID NO: 630 - protein, 5 aa, synthetic construct
  REGION 1..5: note = CD3-VH-n-CDR1
NYYTH

SEQ ID NO: 631 - protein, 17 aa, synthetic construct
  REGION 1..17: note = CD3-VH-n-CDR2
NFYPGDLTVNYDEKFKN

SEQ ID NO: 632 - protein, 8 aa, synthetic construct
  REGION 1..8: note = CD3-VH-n-CDR3
NGNYAMDY

SEQ ID NO: 633 - protein, 5 aa, synthetic construct
  REGION 1..5: note = CD3-VH-n-CDR1
NYYTH

SEQ ID NO: 634 - protein, 17 aa, synthetic construct
  REGION 1..17: note = CD3-VH-n-CDR2
WISPGSGSIKYNEKFKG

SEQ ID NO: 635 - protein, 8 aa, synthetic construct
  REGION 1..8: note = CD3-VH-n-CDR3
NHDYYFDY

SEQ ID NO: 636 - protein, 5 aa, synthetic construct
  REGION 1..5: note = CD3-VH-n-CDR1
NYYTH

SEQ ID NO: 637 - protein, 17 aa, synthetic construct
  REGION 1..17: note = CD3-VH-n-CDR2
WIYPGNGNIKYNEKFMG

SEQ ID NO: 638 - protein, 11 aa, synthetic construct
  REGION 1..11: note = CD3-VH-n-CDR3
RGDYRYAWFTY

SEQ ID NO: 639 - protein, 5 aa, synthetic construct
  REGION 1..5: note = CD3-VH-n-CDR1
SYWMH

SEQ ID NO: 640 - protein, 17 aa, synthetic construct
  REGION 1..17: note = CD3-VH-n-CDR2
WIYPGNGNIKYNEKFMG

SEQ ID NO: 641 - protein, 10 aa, synthetic construct
  REGION 1..10: note = CD3-VH-n-CDR3
DSITNYYFDY

SEQ ID NO: 642 - protein, 5 aa, synthetic construct
  REGION 1..5: note = CD3-VH-n-CDR1
SYYIH

SEQ ID NO: 643 - protein, 17 aa, synthetic construct
  REGION 1..17: note = CD3-VH-n-CDR2
WLYPGNGDTRYNEKFKD

SEQ ID NO: 644 - protein, 10 aa, synthetic construct
  REGION 1..10: note = CD3-VH-n-CDR3
DAYSRYFFDY

SEQ ID NO: 645 - protein, 5 aa, synthetic construct
  REGION 1..5: note = CD3-VH-n-CDR1
SYWMH

SEQ ID NO: 646 - protein, 17 aa, synthetic construct
  REGION 1..17: note = CD3-VH-n-CDR2
NSYPGDLNVNYDEKFKN

SEQ ID NO: 647 - protein, 10 aa, synthetic construct
  REGION 1..10: note = CD3-VH-n-CDR3
DGYSLYFFDF

SEQ ID NO: 648 - protein, 5 aa, synthetic construct
  REGION 1..5: note = CD3-VH-n-CDR1
SCAIS

SEQ ID NO: 649 - protein, 17 aa, synthetic construct
  REGION 1..17: note = CD3-VH-n-CDR2
WLYPGDVSTRYNEKFRD

SEQ ID NO: 650 - protein, 10 aa, synthetic construct
  REGION 1..10: note = CD3-VH-n-CDR3
DSYGSYYFDY

SEQ ID NO: 651 - protein, 5 aa, synthetic construct
  REGION 1..5: note = CD3-VH-n-CDR1
DYYIH

SEQ ID NO: 652 - protein, 17 aa, synthetic construct
  REGION 1..17: note = CD3-VH-n-CDR2
NIYPGGEIINYAEKFKT

SEQ ID NO: 653 - protein, 10 aa, synthetic construct
  REGION 1..10: note = CD3-VH-n-CDR3
DSYGSYYFDY

SEQ ID NO: 654 - protein, 16 aa, synthetic construct
  REGION 1..16: note = CD3-VH-n-CDR2
FMSVTGSAYYANWAKS

SEQ ID NO: 655 - protein, 10 aa, synthetic construct
  REGION 1..10: note = CD3-VH-n-CDR3
DSYGNYFFDY

SEQ ID NO: 656 - protein, 17 aa, synthetic construct
  REGION 1..17: note = CD3-VH-n-CDR2
  VARIANT 9: note = X can be any amino acid
  VARIANT 12..15: note = X can be any amino acid
  VARIANT 17: note = X can be any amino acid
LINPYKGVXTYXXXXKX

SEQ ID NO: 657 - protein, 10 aa, synthetic construct
  REGION 1..10: note = CD3-VH-n-CDR3
DAYSRYFFDY

SEQ ID NO: 658 - protein, 17 aa, synthetic construct
  REGION 1..17: note = CD3-VH-n-CDR2
WIYPGDVSTRFNEKFKG

SEQ ID NO: 659 - protein, 10 aa, synthetic construct
  REGION 1..10: note = CD3-VH-n-CDR3
DSSASYYFDF

SEQ ID NO: 660 - protein, 10 aa, synthetic construct
  REGION 1..10: note = CD3-VH-n-CDR3
DTTGNYFFDY

SEQ ID NO: 661 - protein, 9 aa, synthetic construct
  REGION 1..9: note = CD3-VH-n-CDR3
VGIGSGLNI

SEQ ID NO: 662 - protein, 10 aa, synthetic construct
  REGION 1..10: note = CD3-VH-n-CDR3
  VARIANT 1..2: note = X can be any amino acid
  VARIANT 5..7: note = X can be any amino acid
XXYSXXXFDY

SEQ ID NO: 663 - protein, 10 aa, synthetic construct
  REGION 1..10: note = CD3-VH-n-CDR3
DSYGNYFFDY

SEQ ID NO: 664 - protein, 17 aa, synthetic construct
  REGION 1..17: note = CD3-VL-n-CDR1
KSSQSLLNSRTRKNYLA

SEQ ID NO: 665 - protein, 7 aa, synthetic construct
  REGION 1..7: note = CD3-VL-n-CDR2
WASTRES

SEQ ID NO: 666 - protein, 8 aa, synthetic construct
  REGION 1..8: note = CD3-VL-n-CDR3
TQSFILRT

SEQ ID NO: 667 - protein, 17 aa, synthetic construct
  REGION 1..17: note = CD3-VL-n-CDR1
KSSQSLLNSRTRKNYLA

SEQ ID NO: 668 - protein, 7 aa, synthetic construct
  REGION 1..7: note = CD3-VL-n-CDR2
WTSTRKS

SEQ ID NO: 669 - protein, 8 aa, synthetic construct
  REGION 1..8: note = CD3-VL-n-CDR3
KQSFILRT

SEQ ID NO: 670 - protein, 11 aa, synthetic construct
  REGION 1..11: note = CD3-VL-n-CDR1
RASQDIRNYLN

SEQ ID NO: 671 - protein, 7 aa, synthetic construct
  REGION 1..7: note = CD3-VL-n-CDR2
YTSRLES

SEQ ID NO: 672 - protein, 8 aa, synthetic construct
  REGION 1..8: note = CD3-VL-n-CDR3
KQSFALRT

SEQ ID NO: 673 - protein, 14 aa, synthetic construct
  REGION 1..14: note = CD3-VL-n-CDR1
RSSTGAVTTSNYAN

SEQ ID NO: 674 - protein, 7 aa, synthetic construct
  REGION 1..7: note = CD3-VL-n-CDR2
GTNKRAP

SEQ ID NO: 675 - protein, 8 aa, synthetic construct
  REGION 1..8: note = CD3-VL-n-CDR3
KASFILRT

SEQ ID NO: 676 - protein, 14 aa, synthetic construct
  REGION 1..14: note = CD3-VL-n-CDR1
RSSTGAVTTSNYAN

SEQ ID NO: 677 - protein, 7 aa, synthetic construct
  REGION 1..7: note = CD3-VL-n-CDR2
GTSNRAP

SEQ ID NO: 678 - protein, 8 aa, synthetic construct
  REGION 1..8: note = CD3-VL-n-CDR3
KQSAILRT

SEQ ID NO: 679 - protein, 17 aa, synthetic construct
  REGION 1..17: note = CD3-VL-n-CDR1
KSSQSLLNSRTRKNYLA

SEQ ID NO: 680 - protein, 7 aa, synthetic construct
  REGION 1..7: note = CD3-VL-n-CDR2
WASTRES

SEQ ID NO: 681 - protein, 8 aa, synthetic construct
  REGION 1..8: note = CD3-VL-n-CDR3
TQSFILRT

SEQ ID NO: 682 - protein, 17 aa, synthetic construct
  REGION 1..17: note = CD3-VL-n-CDR1
KSSQSLLSGRTRKNYLA

SEQ ID NO: 683 - protein, 7 aa, synthetic construct
  REGION 1..7: note = CD3-VL-n-CDR2
WASTRES

SEQ ID NO: 684 - protein, 9 aa, synthetic construct
  REGION 1..9: note = CD3-VL-n-CDR3
QQGNTLPWT

SEQ ID NO: 685 - protein, 17 aa, synthetic construct
  REGION 1..17: note = CD3-VL-n-CDR1
KSSQSLLSGRTRKNYLA

SEQ ID NO: 686 - protein, 7 aa, synthetic construct
  REGION 1..7: note = CD3-VL-n-CDR2
WASTRES

SEQ ID NO: 687 - protein, 9 aa, synthetic construct
  REGION 1..9: note = CD3-VL-n-CDR3
ALWYSNLWV

SEQ ID NO: 688 - protein, 17 aa, synthetic construct
  REGION 1..17: note = CD3-VL-n-CDR1
KSSQSVLYSSNNKNYLV

SEQ ID NO: 689 - protein, 7 aa, synthetic construct
  REGION 1..7: note = CD3-VL-n-CDR2
RASTRES

SEQ ID NO: 690 - protein, 9 aa, synthetic construct
  REGION 1..9: note = CD3-VL-n-CDR3
ALWYSTHFV

SEQ ID NO: 691 - protein, 17 aa, synthetic construct
  REGION 1..17: note = CD3-VL-n-CDR1
KSSQSLLNSRTRKNYLA

SEQ ID NO: 692 - protein, 7 aa, synthetic construct
  REGION 1..7: note = CD3-VL-n-CDR2
WASTRES

SEQ ID NO: 693 - protein, 8 aa, synthetic construct
  REGION 1..8: note = CD3-VL-n-CDR3
KQSYILRT

SEQ ID NO: 694 - protein, 17 aa, synthetic construct
  REGION 1..17: note = CD3-VL-n-CDR1
KSSQSLLNSRTRKNYLA

SEQ ID NO: 695 - protein, 7 aa, synthetic construct
  REGION 1..7: note = CD3-VL-n-CDR2
WASTRES

SEQ ID NO: 696 - protein, 8 aa, synthetic construct
  REGION 1..8: note = CD3-VL-n-CDR3
KQSYYLLT

SEQ ID NO: 697 - protein, 17 aa, synthetic construct
  REGION 1..17: note = CD3-VL-n-CDR1
KSSQSLLNSRTRKNYLA

SEQ ID NO: 698 - protein, 7 aa, synthetic construct
  REGION 1..7: note = CD3-VL-n-CDR2
WASTRES

SEQ ID NO: 699 - protein, 8 aa, synthetic construct
  REGION 1..8: note = CD3-VL-n-CDR3
KQSYYLLT

SEQ ID NO: 700 - protein, 17 aa, synthetic construct
  REGION 1..17: note = CD3-VL-n-CDR1
KSSQSLLNSRTRKNYLA

SEQ ID NO: 701 - protein, 7 aa, synthetic construct
  REGION 1..7: note = CD3-VL-n-CDR2
WASTRES

SEQ ID NO: 702 - protein, 9 aa, synthetic construct
  REGION 1..9: note = CD3-VL-n-CDR3
QQYYSVPWT

SEQ ID NO: 703 - protein, 14 aa, synthetic construct
  REGION 1..14: note = CD3-VL-n-CDR1
RSSTGAVTTSNYAN

SEQ ID NO: 704 - protein, 7 aa, synthetic construct
  REGION 1..7: note = CD3-VL-n-CDR2
GTSNRAP

SEQ ID NO: 705 - protein, 8 aa, synthetic construct
  REGION 1..8: note = CD3-VL-n-CDR3
KQSFTLRT

SEQ ID NO: 706 - protein, 17 aa, synthetic construct
  REGION 1..17: note = CD3-VL-n-CDR1
KSSQSLLNIRTRKNCLA

SEQ ID NO: 707 - protein, 7 aa, synthetic construct
  REGION 1..7: note = CD3-VL-n-CDR2
WASTRYS

SEQ ID NO: 708 - protein, 8 aa, synthetic construct
  REGION 1..8: note = CD3-VL-n-CDR3
KQSYTLRT

SEQ ID NO: 709 - protein, 17 aa, synthetic construct
  REGION 1..17: note = CD3-VL-n-CDR1
KSSQSLLNSRTRKNYLA

SEQ ID NO: 710 - protein, 7 aa, synthetic construct
  REGION 1..7: note = CD3-VL-n-CDR2
WASTRES

SEQ ID NO: 711 - protein, 8 aa, synthetic construct
  REGION 1..8: note = CD3-VL-n-CDR3
KQSYTLRT

SEQ ID NO: 712 - protein, 17 aa, synthetic construct
  REGION 1..17: note = CD3-VL-n-CDR1
KSSQSLLNSRTRKNYLA

SEQ ID NO: 713 - protein, 7 aa, synthetic construct
  REGION 1..7: note = CD3-VL-n-CDR2
WASTRES

SEQ ID NO: 714 - protein, 8 aa, synthetic construct
  REGION 1..8: note = CD3-VL-n-CDR3
KQSYILRT

SEQ ID NO: 715 - protein, 17 aa, synthetic construct
  REGION 1..17: note = CD3-VL-n-CDR1
KSSQSLLNSRTRKNYLA

SEQ ID NO: 716 - protein, 7 aa, synthetic construct
  REGION 1..7: note = CD3-VL-n-CDR2
WASTRES

SEQ ID NO: 717 - protein, 9 aa, synthetic construct
  REGION 1..9: note = CD3-VL-n-CDR3
ALWYSTHFV

SEQ ID NO: 718 - protein, 17 aa, synthetic construct
  REGION 1..17: note = CD3-VL-n-CDR1
KSSQSLLNSRTRKNYLA

SEQ ID NO: 719 - protein, 7 aa, synthetic construct
  REGION 1..7: note = CD3-VL-n-CDR2
WASTRES

SEQ ID NO: 720 - protein, 8 aa, synthetic construct
  REGION 1..8: note = CD3-VL-n-CDR3
TQSYTLRT

SEQ ID NO: 721 - protein, 17 aa, synthetic construct
  REGION 1..17: note = CD3-VL-n-CDR1
KSSQSLLNSRTRKNYLA

SEQ ID NO: 722 - protein, 7 aa, synthetic construct
  REGION 1..7: note = CD3-VL-n-CDR2
WASTRES

SEQ ID NO: 723 - protein, 8 aa, synthetic construct
  REGION 1..8: note = CD3-VL-n-CDR3
CQSFILRT

SEQ ID NO: 724 - protein, 17 aa, synthetic construct
  REGION 1..17: note = CD3-VL-n-CDR1
KSSQSLLNSRTRKNYLA

SEQ ID NO: 725 - protein, 7 aa, synthetic construct
  REGION 1..7: note = CD3-VL-n-CDR2
WASTRES

SEQ ID NO: 726 - protein, 8 aa, synthetic construct
  REGION 1..8: note = CD3-VL-n-CDR3
KQSFILRT

SEQ ID NO: 727 - protein, 17 aa, synthetic construct
  REGION 1..17: note = CD3-VL-n-CDR1
KSSQSLLNIRTRKNYLA

SEQ ID NO: 728 - protein, 7 aa, synthetic construct
  REGION 1..7: note = CD3-VL-n-CDR2
WASTRAS

SEQ ID NO: 729 - protein, 8 aa, synthetic construct
  REGION 1..8: note = CD3-VL-n-CDR3
TQSFILRT

SEQ ID NO: 730 - protein, 17 aa, synthetic construct
  REGION 1..17: note = CD3-VL-n-CDR1
KSSQSLLNSRTRKNYLA

SEQ ID NO: 731 - protein, 7 aa, synthetic construct
  REGION 1..7: note = CD3-VL-n-CDR2
WASTRES

SEQ ID NO: 732 - protein, 8 aa, synthetic construct
  REGION 1..8: note = CD3-VL-n-CDR3
KQSFILRT

SEQ ID NO: 733 - protein, 13 aa, synthetic construct
  REGION 1..13: note = CD3-VL-n-CDR1
QASETVYSNNYLA

SEQ ID NO: 734 - protein, 7 aa, synthetic construct
  REGION 1..7: note = CD3-VL-n-CDR2
GVSTLDS

SEQ ID NO: 735 - protein, 8 aa, synthetic construct
  REGION 1..8: note = CD3-VL-n-CDR3
KQSFILRT

SEQ ID NO: 736 - protein, 17 aa, synthetic construct
  REGION 1..17: note = CD3-VL-n-CDR1
KSSQSLLNSRTRKNYLA

SEQ ID NO: 737 - protein, 7 aa, synthetic construct
  REGION 1..7: note = CD3-VL-n-CDR2
WASTRES

SEQ ID NO: 738 - protein, 8 aa, synthetic construct
  REGION 1..8: note = CD3-VL-n-CDR3
CQSFILRT

SEQ ID NO: 739 - protein, 8 aa, synthetic construct
  REGION 1..8: note = CD3-VL-n-CDR3
TQSFILRT

SEQ ID NO: 740 - protein, 8 aa, synthetic construct
  REGION 1..8: note = CD3-VL-n-CDR3
TQSFILRT

SEQ ID NO: 741 - protein, 12 aa, synthetic construct
  REGION 1..12: note = CD3-VL-n-CDR3
AGYKTSSSYAIA

SEQ ID NO: 742 - protein, 8 aa, synthetic construct
  REGION 1..8: note = CD3-VL-n-CDR3
  VARIANT 1..2: note = X can be any amino acid
  VARIANT 4..5: note = X can be any amino acid
XXSXXLRT

SEQ ID NO: 743 - protein, 9 aa, synthetic construct
  REGION 1..9: note = CD3-VL-n-CDR3
CTQSFILRT

SEQ ID NO: 744 - protein, 245 aa, synthetic construct
  REGION 1..245: note = NKp46
STGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSSVEGGSGGSGGSGGSGGVDDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 745 - protein, 25 aa, synthetic construct [sequence omitted]

SEQ ID NO: 746 - protein, 18 aa, synthetic construct
  REGION 1..18: note = FR2
MGWLRQAPGKEREFVVVI

SEQ ID NO: 747 - protein, 18 aa, synthetic construct
  REGION 1..18: note = FR2
MGWFRQAPGKEREFIAVI

SEQ ID NO: 748 - protein, 38 aa, synthetic construct
  REGION 1..38: note = FR3
AYADSVKGRFTISRDNARNTVYLQMNNLKPEDTGVYYC

SEQ ID NO: 749 - protein, 38 aa, synthetic construct
  REGION 1..38: note = FR3
AYADSVKGRFTISRDNARNTVYLQMNSLRPEDTAVYYC

SEQ ID NO: 750 - protein, 38 aa, synthetic construct
  REGION 1..38: note = FR3
AYADSVKGRFTISRDNAKNTVYLQMNSLRPEDTAVYYC

SEQ ID NO: 751 - protein, 38 aa, synthetic construct
  REGION 1..38: note = FR3
SYADSVKGRFTISRDNAKNTLYLQMNSLKPEDTAVYYC

SEQ ID NO: 752 - protein, 38 aa, synthetic construct
  REGION 1..38: note = FR3
SYADSVKGRFTISRDNAKNTLYLQMNSLRPEDTAVYYC

SEQ ID NO: 753 - protein, 11 aa, synthetic construct
  REGION 1..11: note = FR4
WGQGTLVTVSS

SEQ ID NO: 754 - RNA, 7282 nt, synthetic construct
  misc_feature 1..7282: note = SVV
  source 1..7282: mol_type = other RNA
tttgaaatggggggctgggccctgatgcccagtccttcctttccccttccggggggttaaccggctgtgtttgctagaggcacagaggagcaacatccaacctgcttttgtggggaacggtgcggctccaattcctgcgtcgccaaaggtgttagcgcacccaaacggcgcatctaccaatgctattggtgtggtctgcgagttctagcctactcgtttctcccctattcattcactcacgcacaaaaagtgtgttgtaactacaagatttagccctcacacgggatgtgtgataaccgcaagactgactcaagcgcggaaagcgctgtaaccgcatgctgttagtccctttatggctgcgagatggctatccacctcggatcactgaactggagctcgaccctccttagtaagggaaccgagaggccttcttgcaacaagctccgacacagagtccacgtgattgctaccaccatgagtacatggttctcccctctcgacccaggacttctttttgaatatccacggctcgatccagagggtggggcatgatccccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctctccttgttgccatggttcctgaaaccacccttgatgtcaagcccgacggtaaggcaaagagcttacaggagctgaatgaagaacagtgggtggaaatgtctgacgattaccggaccgggaaaaacatgccttttcaggcgcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctctcctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaaggg

SEQ ID NO: 755 - RNA, 7405 nt, synthetic construct
  misc_feature 1..7405: note = CVA
  source 1..7405: mol_type = other RNA
ttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaaatgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccacttttgggtttggtcatcagaacaaagcagtgtacgttgccggttacaagatttgcaactaccacctagcaaccccaagtgatcacttgaatgcaattagtatgttatgggacagggatttaatggtggtggaatctagagcccagggaactgataccatcgccagatgtagttgcaggtgtggagtttactattgtgaatctaggaggaagtactaccctgtcacttttactggcccaacgtttcgattcatggaagcaaacgactactatccagcaagataccagtctcacatgctgatagggtgcggatttgcagaacccggggactgcggtgggatactgaggtgcactcatggggtaattggtatcattactgcaggaggtgaaggggtagtagcctttgctgacattagagacctctgggtgtatgaagaggaggccatggaacagggaataacaagctacatcgaatctctcggcacagcctttggcgcagggttcacccacacaatcagtgagaaagtgactgaattgacaacaatggttaccagcactatcacagaaaaactactgaaaaacttggtgaaaatagtgtcggctctagtgattgttgtgagaaattatgaggacactaccacgatccttgcaacactagcactactcgggtgtgatatatctccttggcaatggttgaagaagaaggcatgtgacttactagagattccttatgtgatgcgccaaggtgatgggtggatgaagaaattcacagaggcgtgcaatgcagctaaaggcttagagtggattagcaacaaaatttccaagtttatagattggttgaagtgtaaaattatcccagacgctaaggacaaggtggaatttctcaccaagttgaaacagctagacatgttggaaaatcaaattgcaaccatccaccaatcttgccccagccaagaacaacaagagattcttttcaacaatgtgagatggctagcagtccagtcccgtcggtttgcaccattatacgctgtggaggcacgccgaattaacaaaatggagagcacaataaacaattatatacagttcaagagcaaacaccgtattgaaccagtatgtatgctcattcatgggtcaccagggacgggtaaatctatagctacttcattaataggtagagcaatagcagagaaggaaagcacatcagtctattcaatgccacctgacccatctcactttgatggctataaacaacaaggggtagtgattatggacgacctaaaccaaaaccccgatggtatggacatgaaactgttttgccaaatggtatcaacagtggagtttattcctccaatggcctcattagaggagaagggcattttgtttacatctgattatgtcctggcttctaccaactctcattcaattgtaccacccacagtggctcacagtgatgccttaaccagacgatttgcatttgatgtggaggtttacacgatgtctgaacattcagtcaaaggcaaactgaatatggccacggccactcaattgtgtaaggattgtccaacacctgcaaattttaaaaagtgttgccctctcgtttgtggaaaggccttgcaattaatggacaggtacaccagacaaaggttcactgtagatgagattaccacattaatcatgaatgagaaaaacagaagggccaatatcggcaattgcatggaagccttgtttcaaggaccattaaggtataaagatttgaagatcgatgtgaagacagttcccccccctgagtgcatcagtgatttgttacaagcagtggattctcaagaggttagggattactgtgagaagaaaggctggatcgttaacgttactagccagattcaactagaaaggaacatcaatagggccatgactatactccaagctgttaccacattcgcagcagtcgcaggagtagtgtatgtaatgtacaaactcttcgccggtcaacagggtgcatacactggcttgccaaacaaaaaacccaatgtccctactatcagagtcgctaaagtccaggggccaggatttgactacgcagtggcaatggcaaaaagaaacatagttactgcaaccaccaccaagggtgaatttaccatgctaggggtgcatgataatgtagcaatattgccaacccatgccgctccaggagaaaccattattattgatgggaaagaagtagagatcctagatgccagagccttagaagatcaagcgggaaccaatcttgagatcaccattattactctaaaaagaaatgagaagtttagagacatcagatcacatattcccacccaaattactgaaactaacgatggagtgttgatcgtgaacactagcaagtaccccaatatgtatgtccccgttggtgctgtgaccgaacagggatatcttaatctcagtggacgtcaaactgctcgcactttaatgtacaactttccaacaagggcaggccagtgcggaggaatcatcacttgtactggcaaagtcattgggatgcatgttggcgggaacggttcacatgggtttgcagcagccctcaagcgatcatacttcactcaaaatcagggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaaccctacctcagtcggattggattgggttatactgttgtaggggtaaatttttctttaattcggag

SEQ ID NO: 756 - protein, 241 aa, synthetic construct
  REGION 1..241: note = human DLL3 BiTE
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 757 - protein, 17 aa, synthetic construct
  REGION 1..17: note = VL-CDR1
KSSQSLLDSEDQKDYLG

SEQ ID NO: 758 - protein, 7 aa, synthetic construct
  REGION 1..7: note = VL-CDR2
WATNRHT

SEQ ID NO: 759 - protein, 9 aa, synthetic construct
  REGION 1..9: note = VL-CDR3
EQYFAYPYT

SEQ ID NO: 760 - protein, 113 aa, synthetic construct
  REGION 1..113: note = VL
DIAIIQSPSSVAVSVGETVTLSCKSSQSLLDSEDQKDYLGWYQQKPGQTPRPLIYWATNRHTGVPDRFTGSGSGTDFTLIISSVQAEDLADYYCEQYFAYPYTFGAGTKLELK

SEQ ID NO: 761 - skipped